(19) United States
(12) Patent Application Publication
(10) Pub. No.: US 2026/0166089 A1
(43) Pub. Date: Jun. 18, 2026

(54) TREATMENT OF CORNEAL ENDOTHELIAL DISEASE

(71) Applicant: Aurion Biotech, Inc., Seattle, WA (US)

(72) Inventors: Arnaud Lacoste, Cambridge, MA (US); Eris Jordan, Philadelphia, PA (US); Sterling Chung, Woodinville, WA (US)

(21) Appl. No.: 19/421,038

(22) Filed: Dec. 16, 2025

Related U.S. Application Data
(60) Provisional application No. 63/735,285, filed on Dec. 17, 2024.
(60) Provisional application No. 63/886,639, filed on Sep. 23, 2025.
(60) Provisional application No. 63/911,038, filed on Nov. 4, 2025.

(51) Int. Cl. A61K 35/30; A61F 9/00; A61K 31/4409; A61P 27/02; C12N 5/00

(52) U.S. Cl. CPC A61K 35/30; A61F 9/0008; A61K 31/4409; A61P 27/02; C12N 5/562

(57) ABSTRACT

Provided are methods of treating or preventing corneal endothelial disease. The present disclosure relates to corneal endothelial cell compositions comprising cultured human corneal endothelial cells and a Rho-associated, coiled-coil containing protein kinase inhibitor, and uses of the compositions for the treatment of corneal endothelial diseases, including bullous keratopathy, corneal edema, corneal leukoma, corneal endothelial inflammation, and corneal dystrophy.

### RELATED APPLICATIONS

This application claims the benefit of U.S. Provisional Application No. 63/735,285, filed Dec. 17, 2024, U.S. Provisional Application No. 63/886,639, filed Sep. 23, 2025, and U.S. Provisional Application No. 63/911,038, filed Nov. 4, 2025, the contents of each of which are herein incorporated by reference in their entirety.

### BACKGROUND

Corneal edema secondary to corneal endothelial dysfunction is a sight-threatening condition affecting millions of people throughout the world. When corneal endothelial cells die or degrade, they do not regenerate in vivo within the cornea. If left untreated, corneal endothelial cell loss can cause corneal edema (swelling) and loss of vision. Although corneal transplants are used to treat corneal endothelial dysfunction, these procedures are complex, invasive and time-consuming. In addition, it is estimated that there is only one healthy donor cornea available for every 70 diseased eyes. As such, there remains a significant unmet need for patients with corneal endothelial disease for alternative treatment options that are effective, straightforward and minimally invasive, without being limited by donor cornea supply.

### SUMMARY OF THE INVENTION

Provided herein is a method for treating a corneal endothelial disease in a human subject in need thereof, comprising administering an effective amount of a corneal endothelial cell (CEC) composition and an effective amount of a Rho-associated, coiled-coil containing protein kinase inhibitor (ROCK inhibitor) to the human subject, wherein the composition comprises allogeneic human CECs, and wherein the corneal endothelial disease is a bullous keratopathy, a corneal edema, a corneal leukoma, a corneal endothelial inflammation, Fuchs' corneal dystrophy, or a corneal dystrophy.

Further provided is a method for treating or preventing a corneal endothelial disease in a human subject in need thereof, comprising administering an effective amount of a corneal endothelial cell (CEC) composition and an effective amount of a ROCK inhibitor to the human subject, wherein the CEC composition comprises allogeneic human CECs, and wherein the human subject has corneal edema secondary to corneal endothelial dysfunction.

In some embodiments, the human subject achieves >15-letter improvement (>3-line gain using the Early Treatment Diabetic Retinopathy Standard (ETDRS) visual acuity test) from baseline in a best corrected visual acuity (BCVA) at six months. In some embodiments, the human subject has a change from baseline in BCVA and in central corneal thickness (CCT) at six months. In certain embodiments, the human subject has no loss from baseline in BCVA of more than 15 letters.

In some embodiments, the CEC composition comprises about 2.5×10⁵ CECs. In other embodiments, the CEC composition comprises about 5×10⁵ CECs. In some embodiments, the CEC composition comprises about 1×10⁶ CECs. In some embodiments, the CEC composition comprises about 2.5×10⁵ to about 1×10⁶ CECs. In some embodiments, the allogeneic human CECs are cultured prior to administration to the human subject. In some embodiments, the CEC composition is administered to an anterior chamber of an eye of the human subject. In some embodiments, the CEC composition is administered to the human subject as a one-time procedure.

In some embodiments, the CEC composition and the ROCK inhibitor are administered as a combination composition. In some embodiments, the combination composition is AURN001. In some embodiments, the CEC composition and the ROCK inhibitor are administered separately. In some embodiments, the CEC composition is neltependocel. In some embodiments, the ROCK inhibitor is Y-27632. In certain embodiments, about 100 microliters of Y-27632 is administered to the human subject.

Also provided herein is a method for treating Fuchs' corneal dystrophy in human subjects in need thereof, comprising administering an effective amount of a corneal endothelial cell (CEC) composition and an effective amount of a ROCK inhibitor to the human subject, wherein the composition comprises allogeneic human CECs, and wherein at least 50% of subjects treated achieve >15-letter improvement (>3-line gain using the Early Treatment Diabetic Retinopathy Standard (ETDRS) visual acuity test).

In some embodiments, the CEC composition comprises about 2.5×10⁵ CECs. In other embodiments, the CEC composition comprises about 5×10⁵ CECs. In further embodiments, the CEC composition comprises about 1×10⁶ CECs. In some embodiments, the CEC composition comprises about 2.5×10⁵ to about 1×10⁶ CECs. In some embodiments, the allogeneic human CECs are cultured prior to administration to the human subjects. In some embodiments, the CEC composition is administered to an anterior chamber of an eye of each of the human subjects. In still other embodiments, the CEC composition is administered to the human subjects as a one-time procedure for each. In some embodiments, the CEC composition and the ROCK inhibitor are administered as a combination composition. In some embodiments, the combination composition is AURN001. In other embodiments, the CEC composition and the ROCK inhibitor are administered separately. In some embodiments, the CEC composition is neltependocel. In further embodiments, the ROCK inhibitor is Y-27632. In certain embodiments, about 100 microliters of Y-27632 is administered to the human subjects.

In some aspects, the present disclosure provides a method for treating a corneal endothelial disease in a human subject in need thereof, comprising administering an effective amount of a corneal endothelial cell (CEC) composition and an effective amount of a ROCK inhibitor to the human subject, wherein the composition comprises allogeneic human CECs, and wherein the corneal endothelial disease is a bullous keratopathy, a corneal edema, a corneal leukoma, a corneal endothelial inflammation, or a corneal dystrophy (optionally, Fuchs' dystrophy).

In some aspects, the present disclosure provides a method for treating or preventing a corneal endothelial disease (optionally, Fuchs' dystrophy) in a human subject in need thereof, comprising administering an effective amount of a corneal endothelial cell (CEC) composition and an effective amount of a ROCK inhibitor to the human subject, wherein the CEC composition comprises allogeneic human CECs, and wherein the human subject has corneal edema secondary to corneal endothelial dysfunction.

In some embodiments, the human subject achieves >15-letter improvement (>3-line gain using the Early Treatment Diabetic Retinopathy Standard (ETDRS) visual acuity test) from baseline in a best corrected visual acuity (BCVA) at six months. In some embodiments, the human subject has a change from baseline in BCVA and in central corneal thickness (CCT) at six months. In some embodiments, the human subject has no loss from baseline in BCVA of more than 15 letters.

In some embodiments, the CEC composition comprises about 2.5×10⁵ CECs. In some embodiments, the CEC composition comprises about 5×10⁵ CECs. In some embodiments, the CEC composition comprises about 1×10⁶ CECs. In some embodiments, the CEC composition comprises about 2.5×10⁵ to about 1×10⁶ CECs. In some embodiments, the allogeneic human CECs are cultured prior to administration to the human subject.

In some embodiments, the CEC composition is administered to an anterior chamber of an eye of the human subject. In some embodiments, the CEC composition is administered to the human subject as a one-time procedure. In some embodiments, the CEC composition and the ROCK inhibitor are administered as a combination composition. In some embodiments, the combination composition is AURN001. In some embodiments, the CEC composition and the Rho-associated, coiled-coil containing protein kinase inhibitor are administered separately. In some embodiments, the CEC composition is neltependocel. In some embodiments, the ROCK inhibitor is Y-27632. In some embodiments, about 100 μM of Y-27632 is administered to the human subject.

In some aspects, the present disclosure provides a method for treating Fuchs' corneal dystrophy in human subjects in need thereof, comprising administering an effective amount of a corneal endothelial cell (CEC) composition and an effective amount of a Rho-associated protein kinase inhibitor to the human subject, wherein the composition comprises allogeneic human CECs, and wherein at least 50% of subjects treated achieve >15-letter improvement (>3-line gain using the Early Treatment Diabetic Retinopathy Standard (ETDRS) visual acuity test).

In some embodiments, the CEC composition comprises about 2.5×10⁵ CECs. In some embodiments, the CEC composition comprises about 5×10⁵ CECs. In some embodiments, the CEC composition comprises about 1×10⁶ CECs. In some embodiments, the CEC composition comprises about 2.5×10⁵ to about 1×10⁶ CECs. In some embodiments, the allogeneic human CECs are cultured prior to administration to the human subjects. In some embodiments, the CEC composition is administered to an anterior chamber of an eye of each of the human subjects. In some embodiments, the CEC composition is administered to the human subjects as a one-time procedure for each. In some embodiments, the CEC composition and the ROCK inhibitor are administered as a combination composition. In some embodiments, the combination composition is AURN001. In some embodiments, the CEC composition and the ROCK inhibitor are administered separately. In some embodiments, the CEC composition is neltependocel. In some embodiments, the Rho-associated, coiled-coil containing protein kinase inhibitor is Y-27632. In some embodiments, about 100 UM of Y-27632 is administered to the human subjects.

In some aspects, the present disclosure provides a method for achieving an improvement from baseline in Best Corrected Visual Acuity (BCVA) at a timepoint post-treatment in a human subject with a corneal endothelial disease in need thereof, the method comprising administering to the human subject a therapeutically effective amount of human corneal endothelial cells (CECs) and a Rho-associated, coiled-coil containing protein kinase inhibitor, wherein the improvement is a ≥10-letter improvement in a Logarithm of the Minimum Angle of Resolution (LogMAR) chart or an equivalent improvement, and wherein the timepoint is at least 6 months post-treatment. In some embodiments, the improvement is a ≥10-letter improvement in the Early Treatment Diabetic Retinopathy Standard (ETDRS) visual acuity test or an equivalent improvement. In some embodiments, the improvement is a ≥15-letter improvement in the Early Treatment Diabetic Retinopathy Standard (ETDRS) visual acuity test or an equivalent improvement. In some embodiments, the equivalent improvement is an improvement in the Snellen chart.

In some aspects, the present disclosure provides a method for achieving a reduced central corneal thickness (CCT) from baseline at a timepoint post-treatment in a human subject with a corneal endothelial disease in need thereof, the method comprising administering to the human subject a therapeutically effective amount of human corneal endothelial cells (CECs) and a ROCK inhibitor, wherein the timepoint is at least 6 months post-treatment.

In some aspects, the present disclosure provides a method for achieving an improved subject-reported quality of life from baseline at a timepoint post-treatment in a human subject with a corneal endothelial disease in need thereof, the method comprising administering to the human subject a therapeutically effective amount of human corneal endothelial cells (CECs) and a ROCK inhibitor, wherein the subject-reported quality of life is measured with Visual Function Questionnaire-25 (VFQ-25) or an equivalent thereof, and wherein the timepoint is at least 6 months post-treatment. In some embodiments, the subject-reported quality of life is measured with VFQ-25.

In some aspects, the present disclosure provides a method for achieving an increased endothelial cell density (ECD) from baseline at a timepoint post-treatment in a human subject with a corneal endothelial disease in need thereof, the method comprising administering to the human subject a therapeutically effective amount of human corneal endothelial cells (CECs) and a ROCK inhibitor, wherein the timepoint is at least 6 months post-treatment.

In some aspects, the present disclosure provides a method for achieving a reduced number of adverse effects of a corneal endothelial disease treatment in a human subject at a timepoint post-treatment, the method comprising administering to the human subject a therapeutically effective amount of human corneal endothelial cells (CECs) and a ROCK inhibitor, wherein the timepoint is at least 6 months post-treatment, and wherein the adverse effects comprise one or more of ocular hypertension, conjunctival hemorrhage, epithelial defect, ocular pain, cystoid macular edema, or corneal abrasion.

In some aspects, the present disclosure provides a method for achieving a reduced number of rescue surgeries in a corneal disease treatment in a human subject at a timepoint post-treatment, the method comprising administering to the human subject a therapeutically effective amount of human corneal endothelial cells (CECs) and a ROCK inhibitor, wherein the timepoint is at least 6 months post-treatment, and wherein the rescue surgeries are selected from Descemet Membrane Endothelial Keratoplasty (DMEK), Descemet Stripping Endothelial Keratoplasty (DSEK), and Penetrating Keratoplasty (PKP).

In some aspects, the present disclosure provides a method for achieving a >3-line gain using the Early Treatment Diabetic Retinopathy Standard (ETDRS) visual acuity test from baseline in a best corrected visual acuity (BCVA) at a timepoint post-treatment in a human subject with a corneal endothelial disease in need thereof, comprising administering to the human subject a therapeutically effective amount of human corneal endothelial cells (CECs) and a ROCK inhibitor, wherein the timepoint is at least 6 months post-treatment.

In some embodiments, the human CECs comprise allogeneic human CECs. In some embodiments, the corneal endothelial disease is a bullous keratopathy, a corneal edema, a corneal leukoma, a corneal endothelial inflammation, or a corneal dystrophy. In some embodiments, the corneal endothelial disease is Fuchs' corneal dystrophy or bullous keratopathy. In some embodiments, the corneal endothelial disease is Fuchs' corneal dystrophy. In some embodiments, the human subject has corneal edema secondary to corneal endothelial dysfunction. In some embodiments, the human subject is within an age group (in years) of 2 and 99. In some embodiments, the human subject is within an age group (in years) of 47 and 99. In some embodiments, the human subject is within an age group (in years) of 52 and 94. In some embodiments, the human subject is within an age group (in years) of 62 and 94.

In some embodiments, the therapeutically effective amount of human CECs comprise about 2.5×10⁵ CECs. In some embodiments, the therapeutically effective amount of human CECs comprises about 5×10⁵ CECs. In some embodiments, the therapeutically effective amount of human CECs comprises about 1×10⁶ CECs. In some embodiments, the therapeutically effective amount of human CECs comprises about 2.5×10⁵ to about 1×10⁶ CECs. In some embodiments, the therapeutically effective amount of human CECs comprise more than 2.5×10⁵ CECs. In some embodiments, the therapeutically effective amount of human CECs comprises more than 5×10⁵ CECs. In some embodiments, the therapeutically effective amount of human CECs comprises more than 1×10⁶ CECs.

In some embodiments, the human CECs are cultured prior to administration to the human subject. In some embodiments, the human CECs and the ROCK inhibitor are administered as a single composition. In some embodiments, the human CECs and the ROCK inhibitor are administered via separate compositions. In some embodiments, the composition(s) are administered to an anterior chamber of an eye of the human subject. In some embodiments, the composition(s) are administered to the human subject as a one-time procedure. In some embodiments, the ROCK inhibitor is Y-27632. In some embodiments, about 9.6 μg of Y-27632 is administered to the human subject. In some embodiments, the Y-27632 is administered at a concentration of about 100 μM. In some embodiments, the human subject has a change from baseline in BCVA and in CCT at six and/or twelve months post-treatment. In some embodiments, the timepoint is at least 7 months post-treatment, at least 8 months post-treatment, at least 9 months post-treatment, at least 10 months post-treatment, at least 11 months post-treatment, or at least 12 months post-treatment.

In some aspects, the present disclosure provides a method for treating a corneal endothelial disease in a human subject in need thereof, comprising administering to the human subject about or more than 1×10⁶ corneal endothelial cells (CECs) and an effective amount of a ROCK inhibitor (optionally, Y-27632), wherein the CECs are allogeneic human CECs, wherein the administering is via a single dose or procedure. In some embodiments, the corneal endothelial disease is Fuchs' dystrophy. In some aspects, the present disclosure provides a method for treating corneal edema associated with a corneal endothelial dysfunction in a human subject in need thereof, comprising administering to the human subject about or more than 1×10⁶ corneal endothelial cells (CECs) and an effective amount of a ROCK inhibitor (optionally, Y-27632), wherein the CECs are allogeneic human CECs, wherein the administering is via a single dose or procedure. In some embodiments, the corneal endothelial dysfunction is Fuchs' dystrophy. In some embodiments, the ROCK inhibitor is Y-27632, and the effective amount of Y-27632 is about 100 μM. In some embodiments, the method is effective to achieve and/or maintain a therapeutic improvement at least or more than 6 months or 12 months post-treatment.

In some aspects, the present disclosure provides a method for treating corneal edema associated with a corneal endothelial dysfunction (optionally, wherein the corneal endothelial dysfunction is Fuchs' dystrophy) in a human subject in need thereof, comprising administering to the human subject about or more than 1×10⁶ corneal endothelial cells (CECs) and 100 μM of Y-27632, wherein the CECs are allogeneic human CECs, wherein administering is via a single dose or procedure, and wherein the method is effective to achieve and/or maintain a therapeutic improvement at least or more than 6 months or 12 months post-treatment.

In some embodiments, the therapeutic improvement is an improvement in Best Corrected Visual Acuity (BCVA) which is a ≥15-letter improvement or >3-line gain in the Early Treatment Diabetic Retinopathy Standard (ETDRS) visual acuity test. In some embodiments, the therapeutic improvement is an improvement in mean BCVA or a reduced central corneal thickness. In some embodiments, the therapeutic improvement is an improvement in the subject-reported quality of life, optionally as measured using Visual Function Questionnaire-25 (VFQ-25). In some embodiments, the therapeutic improvement is a reduced number of needed rescue surgeries for the disease or dysfunction.

In some aspects, the present disclosure provides a method of achieving an improvement in Best Corrected Visual Acuity (BCVA) in a human subject with corneal endothelial disease or dysfunction in need thereof (optionally, wherein the corneal endothelial dysfunction is Fuchs' dystrophy), comprising administering to the human subject about or more than 1×10⁶ corneal endothelial cells (CECs) and an effective amount of a ROCK inhibitor (optionally, 100 μM of Y-27632), wherein the CECs are allogeneic human CECs, wherein the human subject has corneal edema, wherein the improvement is a ≥15-letter improvement or >3-line gain in the Early Treatment Diabetic Retinopathy Standard (ETDRS) visual acuity test, and wherein the improvement is achieved and/or maintained at least or more than 6 months or 12 months post-treatment.

In some embodiments, the administering is via a single dose or procedure. In some embodiments, the administering is to an anterior chamber of an eye of the human subject. In some embodiments, the method does not result in a graft rejection. In some embodiments, the method causes no or substantially no ocular adverse effects (optionally, no or substantially no ocular hypertension, conjunctival hemorrhage, epithelial defect, ocular pain, cystoid macular edema, or corneal abrasion). In some embodiments, the CECs comprise at least 70%, at least 75%, at least 80%, at least 85%, at least 90%, at least 95%, at least 97%, at least 98%, or at least 99% (optionally, at least or more than 80%, 85% or 90%) of human corneal endothelial cells that are CD166 positive, CD105 negative to weakly positive, and CD44 negative to weakly positive; optionally at least or more than 80% or 85% of human corneal endothelial cells that are CD166 positive, CD105 negative, and CD44 negative. In some embodiments, the CECs comprise at least 70%, at least 75%, at least 80%, at least 85%, at least 90%, at least 95%, at least 97%, at least 98%, or at least 99% (optionally at least or more than 80%, 85% or 90%) of human corneal endothelial cells that are CD166 positive, CD105 negative to weakly positive, CD24 negative, and CD44 negative to weakly positive; optionally at least or more than 80% or 85% of human corneal endothelial cells that are CD166 positive, CD105 negative, CD24 negative, and CD44 negative. In some embodiments, the CECs comprise at least 70%, at least 75%, at least 80%, at least 85%, at least 90%, at least 95%, at least 97%, at least 98%, or at least 99% (optionally at least or more than 80%, 85% or 90%) of human corneal endothelial cells that are CD166 positive, CD105 negative to weakly positive, CD26 negative, and CD44 negative to weakly positive; optionally at least or more than 80% or 85% of human corneal endothelial cells that are CD166 positive, CD105 negative, CD26 negative, and CD44 negative.

In some embodiments, the CECs are not in a freshly prepared composition. In some embodiments, the non-fresh CEC composition is administered to the human subject within at most about 1, about 2, or about 3 days since preparation (e.g., formulation of the CEC composition comprising CECs). In some embodiments, the non-fresh CEC composition is stored at 2-8 degrees Celsius (e.g., after preparation and prior to administration). In some embodiments, the non-fresh CEC composition comprises a ROCK inhibitor (optionally, Y-27632 or 100 μM of Y-27632).

In some aspects, the present disclosure provides a method for treating a corneal endothelial disease in a human subject in need thereof, comprising administering an effective amount of a corneal endothelial cell (CEC) composition and an effective amount of a Rho-associated, coiled-coil containing protein kinase (ROCK) inhibitor to the human subject, wherein the CEC composition comprises allogeneic human CECs, the CEC composition is stored at a temperature between about 2° C. and about 8° C. for a period of time before administration, the period of time is about 3 hours to about 72 hours.

In some embodiments, the period of time is about 6 hours to about 48 hours, about 12 hours to about 48 hours, or about 24 hours to about 48 hours. In some embodiments, the ROCK inhibitor is Y-27632, optionally wherein the effective amount of the ROCK inhibitor is about 10 μM to about 500 μM, about 50 μM to about 250 μM, about 80 μM to about 120 μM, or about 100 μM; further optionally wherein the effective amount of the ROCK inhibitor is about 100 μM. In some embodiments, the CEC composition comprises Dulbecco's modified eagle medium (DMEM); optionally wherein the CEC composition further comprises human serum albumin (HSA).

In some embodiments, (i) at least 75% of the allogeneic human CECs are CD166 positive, CD105 negative to weakly positive, and CD44 negative to weakly positive; (ii) at least 75% of the allogeneic human CECs are CD166 positive, CD105 negative, and CD44 negative; (iii) at least 75% of the allogeneic human CECs are CD24 negative, CD166 positive, CD105 negative to weakly positive, and CD44 negative to weakly positive; (iv) at least 75% of the allogeneic human CECs are CD24 negative, CD166 positive, CD105 negative, and CD44 negative; (v) at least 75% of the allogeneic human CECs are CD26 negative, CD166 positive, CD105 negative to weakly positive, and CD44 negative to weakly positive; or (vi) at least 75% of the allogeneic human CECs are CD26 negative, CD166 positive, CD105 negative, and CD44 negative.

In some embodiments, the allogeneic human CECs have at least 70% viability after storage. In some embodiments, the method comprises administering about 2.5×10⁵ to about 1×10⁶ allogeneic human CECs to the human subject; optionally comprising about 1×10⁶ allogeneic human CECs to the human subject.

In some embodiments, the corneal endothelial disease is a bullous keratopathy, a corneal edema, a corneal leukoma, a corneal endothelial inflammation, or a corneal dystrophy (optionally, Fuchs' dystrophy). In some embodiments, the human subject achieves >15-letter improvement (>3-line gain using the Early Treatment Diabetic Retinopathy Standard (ETDRS) visual acuity test) from baseline in a best corrected visual acuity (BCVA) at about 6 months or about 12 months after administration. In some embodiments, the human subject has a reduced central corneal thickness (CCT) from baseline at about 6 months or about 12 months after administration. In some embodiments, the human subject has an increased endothelial cell density (ECD) from baseline at about 6 months or about 12 months after administration.

In some aspects, the present disclosure provides a method for achieving an improvement from baseline in Best Corrected Visual Acuity (BCVA) at a timepoint post-treatment in a human subject with a corneal endothelial disease in need thereof, the method comprising administering to the human subject an effective amount of a corneal endothelial cell (CEC) composition and an effective amount of a Rho-associated, coiled-coil containing protein kinase (ROCK) inhibitor, wherein the improvement is a ≥10-letter improvement in a Logarithm of the Minimum Angle of Resolution (LogMAR) chart or an equivalent improvement, the timepoint post-treatment is at least about 6 months post-treatment, and the CEC composition comprises allogeneic human CECs.

In some embodiments, the CEC composition is stored at a temperature between about 2° C. and about 8° C. for a period of time before administration, and the period of time is about 3 hours to about 72 hours. In some embodiments, the period of time is about 6 hours to about 48 hours, about 12 hours to about 48 hours, or about 24 hours to about 48 hours. In some embodiments, the allogeneic human CECs have at least 70% viability after storage.

In some embodiments, the improvement is a ≥15-letter improvement in a Logarithm of the Minimum Angle of Resolution (LogMAR) chart or an equivalent improvement; optionally wherein the improvement is a ≥15-letter improvement in the Early Treatment Diabetic Retinopathy Standard (ETDRS) visual acuity test. In some embodiments, the method further achieves a reduced central corneal thickness (CCT) from baseline in the human subject at the timepoint post-treatment. In some embodiments, the method further achieves an increased endothelial cell density (ECD) from baseline in the human subject at the timepoint post-treatment. In some embodiments, the timepoint post-treatment is at least 7 months post-treatment, at least 8 months post-treatment, at least 9 months post-treatment, at least 10 months post-treatment, at least 11 months post-treatment, or at least 12 months post-treatment.

In some embodiments, the ROCK inhibitor is Y-27632, optionally wherein the effective amount of the ROCK inhibitor is about 10 μM to about 500 μM, about 50 μM to about 250 μM, about 80 μM to about 120 μM, or about 100 μM; further optionally wherein the effective amount of the ROCK inhibitor is about 100 μM. In some embodiments, the CEC composition comprises Dulbecco's modified eagle medium (DMEM); optionally wherein the CEC composition further comprises human serum albumin (HSA).

In some embodiments, (i) at least 75% of the allogeneic human CECs are CD166 positive, CD105 negative to weakly positive, and CD44 negative to weakly positive; (ii) at least 75% of the allogeneic human CECs are CD166 positive, CD105 negative, and CD44 negative; (iii) at least 75% of the allogeneic human CECs are CD24 negative, CD166 positive, CD105 negative to weakly positive, and CD44 negative to weakly positive; (iv) at least 75% of the allogeneic human CECs are CD24 negative, CD166 positive, CD105 negative, and CD44 negative; (v) at least 75% of the allogeneic human CECs are CD26 negative, CD166 positive, CD105 negative to weakly positive, and CD44 negative to weakly positive; or (vi) at least 75% of the allogeneic human CECs are CD26 negative, CD166 positive, CD105 negative, and CD44 negative.

In some embodiments, the method comprises administering about 2.5×10⁵ to about 1×10⁶ allogeneic human CECs to the human subject; optionally comprising about 1×10⁶ allogeneic human CECs to the human subject. In some embodiments, the corneal endothelial disease is a bullous keratopathy, a corneal edema, a corneal leukoma, a corneal endothelial inflammation, or a corneal dystrophy (optionally, Fuchs' dystrophy).

In some aspects, the present disclosure provides a method for treating a corneal endothelial disease in a plurality of human subjects in need thereof, the method comprising administering to the plurality of human subjects an effective amount of a corneal endothelial cell (CEC) composition and an effective amount of a Rho-associated protein kinase (ROCK) inhibitor, wherein the CEC composition comprises allogeneic human CECs, and at least 50% of the plurality of subjects treated achieve >15-letter improvement (>3-line gain) from baseline in Best Corrected Visual Acuity (BCVA) using the Early Treatment Diabetic Retinopathy Standard (ETDRS) visual acuity test at a timepoint post-treatment.

In some embodiments, the CEC composition is stored at a temperature between about 2° C. and about 8° C. for a period of time before administration, and the first period of time is about 3 hours to about 72 hours. In some embodiments, the period of time is about 6 hours to about 48 hours, about 12 hours to about 48 hours, or about 24 hours to about 48 hours. In some embodiments, the allogeneic human CECs have at least 70% viability after storage. In some embodiments, the timepoint post-treatment is at least 6 months post-treatment, at least 7 months post-treatment, at least 8 months post-treatment, at least 9 months post-treatment, at least 10 months post-treatment, at least 11 months post-treatment, or at least 12 months post-treatment.

In some embodiments, the ROCK inhibitor is Y-27632, optionally wherein the effective amount of the ROCK inhibitor is about 10 μM to about 500 μM, about 50 μM to about 250 μM, about 80 μM to about 120 μM, or about 100 μM; further optionally wherein the effective amount of the ROCK inhibitor is about 100 μM. In some embodiments, the CEC composition comprises Dulbecco's modified eagle medium (DMEM); optionally wherein the CEC composition further comprises human serum albumin (HSA).

In some embodiments, (i) at least 75% of the allogeneic human CECs are CD166 positive, CD105 negative to weakly positive, and CD44 negative to weakly positive; (ii) at least 75% of the allogeneic human CECs are CD166 positive, CD105 negative, and CD44 negative; (iii) at least 75% of the allogeneic human CECs are CD24 negative, CD166 positive, CD105 negative to weakly positive, and CD44 negative to weakly positive; (iv) at least 75% of the allogeneic human CECs are CD24 negative, CD166 positive, CD105 negative, and CD44 negative; (v) at least 75% of the allogeneic human CECs are CD26 negative, CD166 positive, CD105 negative to weakly positive, and CD44 negative to weakly positive; or (vi) at least 75% of the allogeneic human CECs are CD26 negative, CD166 positive, CD105 negative, and CD44 negative.

In some embodiments, the method comprises administering about 2.5×10⁵ to about 1×10⁶ allogeneic human CECs to each of the plurality of human subjects; optionally comprising about 1×10⁶ allogeneic human CECs to each of the plurality of human subjects. In some embodiments, the corneal endothelial disease is a bullous keratopathy, a corneal edema, a corneal leukoma, a corneal endothelial inflammation, or a corneal dystrophy (optionally, Fuchs' dystrophy).

In some embodiments, the allogeneic human CECs are in a suspension form or are not substantially adherent to a surface of a container of the CEC composition.

### BRIEF DESCRIPTION OF THE DRAWINGS

FIG. 1 shows the treatment groups and timelines of the CLARA study.

FIG. 2 shows baseline BCVA and CCT baseline values in the CLARA study and other studies to treat ocular disease.

FIG. 3 shows the demographics of subjects enrolled in the each treatment group and overall in the CLARA study.

FIG. 4 shows the number of rescue surgeries per treatment group performed by month 3 and month 6 of the CLARA study.

FIG. 5 shows the proportion of responders with a >15-letter improvement from baseline in BCVA at 6 months (FAS), the primary efficacy endpoint outcome for each group in the CLARA study.

FIG. 6 shows the mean BCVA and LS mean change from baseline BCVA at 6 months (FAS, LOCF), a secondary efficacy endpoint outcome of the CLARA study.

FIG. 7 shows the mean CCT and LS mean change from baseline CCT at 6 months (FAS, LOCF), a secondary efficacy endpoint outcome of the CLARA study.

FIGS. 8A-8E show patient reported VFQ-25 scores in the CLARA study. FIG. 8A shows the composite VFQ-25 score. FIGS. 8B-8E show various components of the score as indicated. The VFQ-25 (Visual Function Questionnaire-25) is a validated and standardized tool designed to assess the impact of visual impairment on a person's daily life and well-being, focusing on vision-related quality of life. 100=best possible score, 0=worst possible score.

FIG. 9 shows a post-hoc BCVA responder analysis in the CLARA study to determine the frequency and proportion of subjects with BCVA improvements, declines, or no change at 6 months.

FIGS. 10A-10C show results from safety assessments in the CLARA study. FIG. 10A shows treatment emergent events. FIG. 10B shows the most common adverse events. FIG. 10C shows mean intraocular pressure over time.

FIG. 11 shows a proposed Phase III study design.

FIG. 12 shows the treatment groups and timelines of the CLARA study as evaluated at 12-month post-treatment.

FIG. 13 shows the proportion of subjects with a >15-letter improvement from baseline in BCVA at 12 months post-treatment in the CLARA study in each treatment group (100 μM Y-27632, 1×10⁶ CECs, 2.5×10⁵ CECs with 100 μM Y-27632, 5×10⁵ CECs with 100 μM Y-27632, or 1×10⁶ CECs with 100 μM Y-27632).

FIGS. 14A-14B show mean BCVA scores in ETDRS visual acuity test (FIG. 14A) and mean BCVA changes from baseline (FIG. 14B) at 12 months post-treatment in each treatment group (100 μM Y-27632, 1×10⁶ CECs, 2.5×10⁵ CECs with 100 μM Y-27632, 5×10⁵ CECs with 100 μM Y-27632, or 1×10⁶ CECs with 100 μM Y-27632) in the CLARA study. Error bars represent standard error.

FIGS. 15A-15B show mean central corneal thickness (CCT) (FIG. 15A) and mean CCT changes from baseline (FIG. 15B) at 12 months post-treatment in each treatment group (100 μM Y-27632, 1×10⁶ CECs, 2.5×10⁵ CECs with 100 μM Y-27632, 5×10⁵ CECs with 100 μM Y-27632, or 1×10⁶ CECs with 100 μM Y-27632) in the CLARA study. Error bars represent standard error.

FIGS. 16A-16B show mean BCVA (FIG. 16A) and mean CCT (FIG. 16B) in subjects with a >15-letter improvement from baseline in BCVA at 12 months post-treatment in the treatment group of 1×10⁶ CECs with 100 μM Y-27632 in the CLARA study.

FIGS. 17A-17C show BCVA (FIG. 17A), CCT (FIG. 17B), and corneal endothelial cell density (ECD) (FIG. 17C) changes across timepoints within 12 months post-treatment in a representative subject that received 1×10⁶ CECs with 100 μM Y-27632 in the CLARA study.

FIG. 18 shows subject reported composite VFQ-25 scores at baseline, 3 months post-treatment, 6 months post-treatment, and 12 months post-treatment in each treatment group (100 UM Y-27632, 1×10⁶ CECs, 2.5×10⁵ CECs with 100 μM Y-27632, 5×10⁵ CECs with 100 μM Y-27632, or 1×10⁶ CECs with 100 μM Y-27632) in the CLARA study.

FIG. 19 shows the number of rescue surgeries per treatment group performed by month 3, month 6, and month 12 post-treatment in the CLARA study.

FIG. 20 shows the intraocular pressure across all treatment groups from baseline to month 12 post-treatment in the CLARA study.

FIG. 21 shows the treatment groups and timelines of the ESCALÓN study.

FIG. 22 shows the demographics of subjects enrolled in each treatment group and the overall characteristics in the ESCALÓN study, and baseline BCVA, BAT, CCT, and ECD measurements in the ESCALÓN study.

FIG. 23 shows overall results from safety assessments in the ESCALÓN study.

FIG. 24 shows the nature and incidence of treatment emergent adverse events (TEAEs) in the study eyes from safety assessments in the ESCALÓN study.

FIG. 25 shows intraocular pressure (IOP) changes in subjects with elevated IOP in the ESCALÓN study.

FIG. 26 shows changes in CCT from baseline through month 12 in the ESCALÓN study. Data were separated by treatment groups, showing a trend toward greater improvement with higher Y-27632 doses. Mean changes are plotted with error bars representing the SD.

FIG. 27 shows changes in BCVA from baseline through month 12 in the ESCALÓN study. The data were separated by treatment groups. Mean changes are plotted with error bars representing the SD.

FIG. 28 shows percent of responders with study eye BCVA improvement ≥0.3 LogMAR in the ESCALÓN study.

FIG. 29 shows changes in Brightness Acuity Testing (BAT) from baseline through month 12 in the ESCALÓN study. The data were separated by treatment groups. Mean changes are plotted with error bars representing the SD.

FIG. 30 shows ECD measurement by treatment group and time point in the ESCALÓN study.

### DETAILED DESCRIPTION OF INVENTION

The present disclosure provides, among other things, corneal endothelial cell (CEC) compositions, and methods and uses of the CEC compositions disclosed herein, e.g., in treatment of a corneal endothelial disease in a human subject in need thereof.

### I. Definitions

In order for the present disclosure to be more readily understood, certain terms are first defined below. Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

As used herein, “a”, “an”, and “the” refer to one or to more than one (i.e., to at least one) of the grammatical object of the article. By way of example, “an element” discloses embodiments of exactly one element and embodiments including more than one element.

As used herein, the term “about”, when used in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referenced value.

As used herein, the term “administration” or “administering” typically refers to administration of a composition to a subject or system to achieve delivery of an agent that is, or is included in, the composition.

Two events or entities are “associated” with one another, as that term is used herein, if the presence, level and/or form of one is correlated with that of the other. For example, a particular entity (e.g., polypeptide, genetic signature, metabolite, microbe, etc.) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact (e.g., covalently or non-covalently), directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, or a combination thereof.

As used herein, the term “between” refers to content that falls between indicated upper and lower, or first and second, boundaries (or “bounds”), inclusive of the boundaries. Similarly, the term “from”, when used in the context of a range of values, indicates that the range includes content that falls between indicated upper and lower, or first and second, boundaries, inclusive of the boundaries.

As used herein, the term “corneal endothelial disease” refers to a disease affecting corneal endothelial cells. Non-limiting examples of corneal endothelial diseases include bullous keratopathy, corneal endothelial dystrophies (e.g., cornea guttata, Fuchs endothelial corneal dystrophy, posterior polymorphous corneal dystrophy, and congenital hereditary corneal endothelial dystrophy), iridocorneal endothelial syndrome, viral diseases (e.g., cytomegalovirus endotheliitis and herpetic endotheliitis), exfoliation syndrome, and corneal endothelial graft rejection; as well as inflammation or physical damage associated with external factors, such as keratouveitis, interstitial keratitis, corneal endotheliitis, corneal endothelial cell loss after corneal transplantation, corneal injury after intraocular surgery (e.g., cataract surgery, vitreous surgery, glaucoma surgery), corneal injury induced by glaucomatous attack, corneal injury caused by long-term contact lens use, corneal trauma, corneal edema, and intrapartum corneal trauma.

As used herein, the term “Fuchs' corneal dystrophy” or “Fuchs' endothelial dystrophy” or “Fuchs endothelial corneal dystrophy”, used interchangeably herein, refers to a condition whereby fluid builds up in the cornea of the eye of a subject.

As used herein, the term “corneal endothelial cell” or “CEC” refers to a cell derived from a corneal endothelium layer or a cell that otherwise has functional and biochemical characteristics of cells in the corneal endothelium layer, including but not limited to primary culture cells, cultured or subcultured cells, and cells induced to differentiate from undifferentiated cells such as stem cells (e.g., embryonic stem cells or induced pluripotent stem cells (iPSCs)). A cornea is one of the lamellar tissues constituting an eye. In humans, the cornea is composed of five layers, corneal epithelium, Bowman's membrane (external boundary), Lamina propria, Descemet's membrane (internal boundary), and corneal endothelium, in order from the outside (body surface). The corneal endothelium is a single layer of cells that covers the posterior cornea. Markers for characterizing CECs and methods of identifying CECs are known in the art (See e.g., Hamuro J, et al. Invest Ophthalmol Vis Sci. 2016 Aug. 1; 57 (10): 4385-92. doi: 10.1167/iovs.16-19771. PMID: 27564520; Wongvisavavit, R., et al (2021). Regenerative medicine, 16 (09), 871-891).

As used herein, a cell derived from corneal endothelial tissue is referred to as “corneal endothelial tissue derived cell.” A cell that becomes a corneal endothelial cell by differentiation is referred to as a “corneal endothelial progenitor cell.”

As used herein, the term “cultured” refers to cells that have been maintained, propagated, expanded, passaged, and/or otherwise manipulated outside a living organism in an artificial environment (e.g., in vitro or ex vivo) for any period sufficient to preserve viability and/or alter number, phenotype, or functional state. “Cultured” cells include primary cells isolated from donor tissue and maintained in medium; cells expanded through one or more passages; cells conditioned by exposure to defined medium components (e.g., serum, serum-free supplements, growth factors, ROCK inhibitors), extracellular matrix coatings, feeder layers, or co-culture; and cells collected or harvested following culture (e.g., post-culture harvest from a vessel or bioreactor). The term “cultured” is not limited by passage number, seeding density, vessel type, substrate, oxygen tension, or duration of maintenance, and does not require that the cells actively divide.

As used herein, the term “expanding” refers to a cell culture process that includes transferring (i.e., passaging) cells into a new culture vessel at a lower cell density in order to permit further growth of the cells.

As used herein, “expression” refers individually and/or cumulatively to one or more biological process that result in production from a nucleic acid sequence of an encoded agent (i.e., an expression product), such as an RNA and/or a polypeptide. Expression specifically includes either or both of transcription and translation. A nucleic acid or cell that produces the encoded agent can be said to express the encoded agent.

As used herein, the terms “improve”, “increase”, “inhibit”, “decrease” and “reduce”, and grammatical equivalents thereof, indicate qualitative or quantitative difference from a reference.

As used here, the term “or” is understood as “and/or” and includes both inclusive and exclusive use of the term unless clearly indicated otherwise by context.

As used herein, the term “treating” in the context of a corneal endothelial disease, refers to therapeutic treatment in order to alleviate one or more symptoms of a corneal endothelial disease. A subject who is treated according to the methods described herein, has a corneal endothelial disease, such that treatment alleviates or slows progression of one or more symptoms of the disease. Exemplary symptoms of the corneal endothelial disease include, but are not limited to, loss of vision, blindness, mechanical disruption of the visual axis, opacification and decreased vision, or an otherwise impairment of visual function. In some embodiments, the present disclosure provides a method of reducing or ameliorating these symptoms.

A “therapeutically effective amount,” an “effective amount,” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated. The skilled worker can readily determine the effective amount for a given situation by routine experimentation.

The term “preventing” as used herein in the context of a corneal endothelial disease refers to a prophylactic measure whereby symptoms of a corneal endothelial disease are inhibited in a subject who may be at risk of developing a corneal endothelial disease or who has been diagnosed with a corneal endothelial disease but is not yet showing certain symptoms, e.g., is not yet showing vision loss. In some embodiments, the disclosure provides a method of preventing vision loss in a patient who is at risk of a corneal endothelial disease or who has been diagnosed with a corneal endothelial disease who is at risk of vision loss.

As used herein, the term “subject” refers to an organism, typically a mammal (e.g., a human, rat, or mouse). In some embodiments, a subject is suffering from a disease, disorder or condition. In some embodiments, a subject is susceptible to a disease, disorder, or condition. In some embodiments, a subject displays one or more symptoms or characteristics of a disease, disorder or condition. In some embodiments, a subject is not suffering from a disease, disorder or condition. In some embodiments, a subject does not display any symptom or characteristic of a disease, disorder, or condition. In some embodiments, a subject has one or more features characteristic of susceptibility to or risk of a disease, disorder, or condition. In some embodiments, a subject is a subject that has been tested for a disease, disorder, or condition, and/or to whom therapy has been administered. A subject administered an agent associated with treatment of a disease, disorder, or condition with which the subject is associated can be referred to as a subject in need of the agent, i.e., as a subject in need thereof. The terms “patient” and “subject” are used interchangeably herein. Preferably, the patient is a human patient.

### II. Methods of Treating Corneal Disease

Provided herein are methods of treating or preventing a corneal endothelial disease in a subject in need thereof by administering an effective amount of a corneal endothelial cell (CEC) composition and an effective amount of a Rho-associated, coiled-coil containing protein kinase (ROCK) inhibitor to the human subject. The CECs are allogeneic human CECs.

Also provided herein are methods for achieving an improvement from baseline in Best Corrected Visual Acuity (BCVA) at a timepoint post-treatment (e.g., at least 6 months post-treatment) in a human subject with a corneal endothelial disease in need thereof, the method comprising administering to the human subject a therapeutically effective amount of human corneal endothelial cells (CECs) (e.g., CECs of a CEC composition disclosed herein) and a Rho-associated, coiled-coil containing protein kinase inhibitor. BCVA is the sharpest, clearest vision a subject can achieve when using the optimal prescription lenses (glasses or contact lenses) to correct any refractive errors in their eyes. It represents the maximum visual acuity attainable for an individual, taking into account the best possible optical correction. An improvement from baseline in BCVA refers to the increase in a subject's visual acuity, measured with optimal corrective lenses, compared to their initial (baseline) measurement before the treatment. The baseline is the BCVA recorded at the start of a medical procedure. Improvement is typically assessed by comparing this initial value to subsequent BCVA measurements taken after the medical procedure. The improvement may be a ≥5-letter improvement in a Logarithm of the Minimum Angle of Resolution (LogMAR) chart or an equivalent improvement. A LogMAR chart is a chart of rows of letters used by ophthalmologists, orthoptists, optometrists, and vision scientists to estimate visual acuity. A LogMAR chart allows for a logarithmic or proportional change in letter size and spacing across rows of letters, and can be scored with reference to the logarithm of the minimum angle of resolution. Each line on the chart contains the same number of letters (usually 5), and the size of the letters decreases in equal logarithmic steps from one line to the next. Examples of LogMAR charts include, without limitation, an Early Treatment Diabetic Retinopathy Standard (ETDRS) chart or a Bailey-Lovie chart. Commonly used alternatives to and equivalents of a LogMAR chart include, without limitation, a Snellen chart. Each letter on a LogMAR chart is worth 0.02 LogMAR units. Each line represents a 0.1 LogMAR change. The total score is calculated based on the number of letters correctly identified, not just the last line read. A 5-letter improvement means the subject can correctly identify 5 more letters than at baseline (e.g., before treatment). Since each letter is worth 0.02 LogMAR, a 5-letter improvement equals a 0.1 LogMAR decrease (improvement) in visual acuity score. A 5-letter improvement is equivalent to gaining one full line on the LogMAR chart. In some embodiments, the improvement is a ≥10-letter improvement in a LogMAR chart (e.g., an ETDRS test) or an equivalent improvement. In some embodiments, the improvement is a ≥15-letter improvement in a LogMAR chart (e.g., an ETDRS test) or an equivalent improvement. In some embodiments, the improvement is a ≥20-letter improvement in a LogMAR chart (e.g., an ETDRS test) or an equivalent improvement.

In other aspects, the present disclosure provides a method for achieving a >3-line gain using the Early Treatment Diabetic Retinopathy Standard (ETDRS) visual acuity test from baseline in a best corrected visual acuity (BCVA) at a timepoint post-treatment in a human subject with a corneal endothelial disease in need thereof, comprising administering to the human subject a therapeutically effective amount of human corneal endothelial cells (CECs) (e.g., CECs of a CEC composition disclosed herein) and a Rho-associated, coiled-coil containing protein kinase inhibitor, wherein the timepoint is at least 6 months post-treatment. A >3-line gain in BCVA means that, after treatment, the subject can read at least three more lines on a standardized visual acuity chart (such as the Snellen or LogMAR/ETDRS chart) than they could at baseline (before treatment), when using their best possible optical correction (glasses or contact lenses). On a visual acuity chart, each horizontal row of letters is called a “line.” On the ETDRS/LogMAR chart, this is equivalent to a gain of 15 or more letters (since each line=5 letters; 3 lines=15 letters).

In further aspects, the present disclosure provides a method for achieving a reduced central corneal thickness (CCT) from baseline at a timepoint post-treatment in a human subject with a corneal endothelial disease in need thereof, the method comprising administering to the human subject a therapeutically effective amount of human corneal endothelial cells (CECs) (e.g., CECs of a CEC composition disclosed herein) and a Rho-associated, coiled-coil containing protein kinase inhibitor, wherein the timepoint is at least 6 months post-treatment. A reduced central corneal thickness (CCT) from baseline means that the thickness of the central part of the cornea (e.g., measured in micrometers or microns, μm) has decreased compared to the initial (baseline) measurement taken before treatment. CCT may be measured using devices such as ultrasound pachymetry, optical coherence tomography (OCT), or Scheimpflug imaging. The purpose of administering CECs to a subject's eye is to restore or improve the function of the corneal endothelium. The endothelium is responsible for maintaining corneal clarity by actively pumping excess fluid out of the corneal stroma, thereby keeping the cornea thin, clear, and properly hydrated. A thickened cornea (increased CCT) usually indicates corneal edema (swelling), which is a sign of endothelial dysfunction or failure. This is common in conditions like Fuchs' endothelial dystrophy or after significant endothelial cell loss. A thinner cornea (reduced CCT) can occur after successful resolution of corneal edema. Patients with corneal endothelial failure often have increased CCT due to corneal swelling/edema. The goal is for the CCT to decrease toward the normal range as the new endothelial cells begin to function, pump out excess fluid, and restore corneal deturgescence (dehydration and clarity).

In yet further aspects, the present disclosure provides a method for achieving an improved subject-reported quality of life from baseline at a timepoint post-treatment (e.g., at least 6 months post-treatment) in a human subject with a corneal endothelial disease in need thereof, the method comprising administering to the human subject a therapeutically effective amount of human corneal endothelial cells (CECs) (e.g., CECs of a CEC composition disclosed herein) and a Rho-associated, coiled-coil containing protein kinase inhibitor. The subject-reported quality of life may be measured with Visual Function Questionnaire-25 (VFQ-25) or an equivalent thereof. An improved subject-reported quality of life means that, according to the subject's own assessment, their daily life, well-being, and ability to function have become better as a result of improved vision or vision-related health. This improvement is measured using standardized questionnaires or surveys that ask patients about how their vision affects their activities, independence, emotions, and social participation. These are called vision-related quality of life (VRQOL) or vision-specific patient-reported outcome measures (PROMs). For example, NEI VFQ-25 (National Eye Institute Visual Function Questionnaire-25) covers aspects like general vision, near and distance activities, social functioning, mental health, dependency, driving, and peripheral vision. VF-14 (Visual Function Index) focuses on the ability to perform daily activities requiring vision. Higher scores generally indicate better vision-related quality of life. Examples of improvement are being able to read, drive, or recognize faces more easily, feeling less dependent on others, experiencing less frustration, anxiety, or depression related to vision, or participating more in social or work activities.

In some aspects, the present disclosure provides a method for achieving an increased endothelial cell density (ECD) from baseline at a timepoint post-treatment in a human subject with a corneal endothelial disease in need thereof, the method comprising administering to the human subject a therapeutically effective amount of human corneal endothelial cells (CECs) (e.g., CECs of a CEC composition disclosed herein) and a Rho-associated, coiled-coil containing protein kinase inhibitor, wherein the timepoint is at least 6 months post-treatment. An increased endothelial cell density (ECD) from baseline means that the number of corneal endothelial cells per square millimeter (cells/mm²) measured after the treatment is higher than the number measured at the initial (baseline) assessment. ECD is typically measured using specular or confocal microscopy, which allows visualization and counting of the endothelial cells on the inner surface of the cornea.

In other aspects, the present disclosure provides a method for achieving a reduced number of adverse effects of a corneal endothelial disease treatment in a human subject at a timepoint post-treatment, the method comprising administering to the human subject a therapeutically effective amount of human corneal endothelial cells (CECs) (e.g., CECs of a CEC composition disclosed herein) and a Rho-associated, coiled-coil containing protein kinase (ROCK) inhibitor, wherein the timepoint is at least 6 months post-treatment, and wherein the adverse effects comprise one or more of ocular hypertension, conjunctival hemorrhage, epithelial defect, ocular pain, cystoid macular edema, or corneal abrasion. A reduced number of adverse effects means that, following the treatment for corneal endothelial disease, a subject experiences fewer unwanted or harmful side effects compared to a previous standard, baseline, or control group. This is a key indicator of improved safety and tolerability of the treatment. In the context of corneal endothelial disease treatments, common adverse effects may include:
1. Ocular hypertension: Elevated intraocular pressure, which can increase the risk of glaucoma;
2. Conjunctival hemorrhage: Bleeding under the conjunctiva, often seen as a red patch on the white of the eye;
3. Epithelial defect: Loss or damage to the corneal surface layer, which can delay healing and increase infection risk;
4. Ocular pain: Discomfort or pain in the eye, which can range from mild to severe;
5. Cystoid macular edema: Swelling of the central retina (macula), which can impair vision; or
6. Corneal abrasion: Scratching or injury to the corneal surface.

In some aspects, the present disclosure provides a method for achieving a reduced number of rescue surgeries in a corneal disease treatment in a human subject at a timepoint post-treatment, the method comprising administering to the human subject a therapeutically effective amount of human corneal endothelial cells (CECs) (e.g., CECs of a CEC composition disclosed herein) and a Rho-associated, coiled-coil containing protein kinase inhibitor, wherein the timepoint is at least 6 months post-treatment, and wherein the rescue surgeries are selected from Descemet Membrane Endothelial Keratoplasty (DMEK), Descemet Stripping Endothelial Keratoplasty (DSEK), and Penetrating Keratoplasty (PKP). A reduced number of rescue surgeries means that, following the treatment, fewer patients require additional, unplanned surgical interventions (called “rescue surgeries”) to address complications, treatment failure, or inadequate response to the treatment. This is a key indicator of the effectiveness and safety of the treatment. In the context of corneal disease treatments, rescue surgeries may include:
1. A procedure (e.g., DMEK, DSEK, or PKP);
2. Surgical interventions to address complications (e.g., graft detachment requiring re-bubbling, graft failure, persistent corneal edema, or infection). In clinical trials and real-world studies, the number and percentage of patients requiring rescue surgery after the treatment are tracked and compared between treatment groups.

In some embodiments, the human subject of the present disclosure is within an age group (in years) of 2 and 99. In some embodiments, the human subject is within an age group (in years) of 47 and 99. In some embodiments, the human subject is within an age group (in years) of 52 and 94. In some embodiments, the human subject is within an age group (in years) of 62 and 94. In some embodiments, the human subject is within an age group (in years) of 2 and 99, of 2 and 94, of 2 and 89, of 2 and 84, of 2 and 79, of 2 and 74, of 2 and 69, of 2 and 64, of 2 and 59, of 2 and 54, of 2 and 49, of 2 and 44, of 2 and 39, of 2 and 34, of 2 and 29, of 2 and 24, of 2 and 19, of 2 and 14, of 2 and 9, or of 2 and 4. In some embodiments, the human subject is within an age group (in years) of 7 and 99, of 7 and 94, of 7 and 89, of 7 and 84, of 7 and 79, of 7 and 74, of 7 and 69, of 7 and 64, of 7 and 59, of 7 and 54, of 7 and 49, of 7 and 44, of 7 and 39, of 7 and 34, of 7 and 29, of 7 and 24, of 7 and 19, of 7 and 14, or of 7 and 9. In some embodiments, the human subject is within an age group (in years) of 12 and 99, of 12 and 94, of 12 and 89, of 12 and 84, of 12 and 79, of 12 and 74, of 12 and 69, of 12 and 64, of 12 and 59, of 12 and 54, of 12 and 49, of 12 and 44, of 12 and 39, of 12 and 34, of 12 and 29, of 12 and 24, of 12 and 19, or of 12 and 14. In some embodiments, the human subject is within an age group (in years) of 17 and 99, of 17 and 94, of 17 and 89, of 17 and 84, of 17 and 79, of 17 and 74, of 17 and 69, of 17 and 64, of 17 and 59, of 17 and 54, of 17 and 49, of 17 and 44, of 17 and 39, of 17 and 34, of 17 and 29, of 17 and 24, or of 17 and 19. In some embodiments, the human subject is within an age group (in years) of 22 and 99, of 22 and 94, of 22 and 89, of 22 and 84, of 22 and 79, of 22 and 74, of 22 and 69, of 22 and 64, of 22 and 59, of 22 and 54, of 22 and 49, of 22 and 44, of 22 and 39, of 22 and 34, of 22 and 29, or of 22 and 24. In some embodiments, the human subject is within an age group (in years) of 27 and 99, of 27 and 94, of 27 and 89, of 27 and 84, of 27 and 79, of 27 and 74, of 27 and 69, of 27 and 64, of 27 and 59, of 27 and 54, of 27 and 49, of 27 and 44, of 27 and 39, of 27 and 34, or of 27 and 29. In some embodiments, the human subject is within an age group (in years) of 32 and 99, of 32 and 94, of 32 and 89, of 32 and 84, of 32 and 79, of 32 and 74, of 32 and 69, of 32 and 64, of 32 and 59, of 32 and 54, of 32 and 49, of 32 and 44, of 32 and 39, or of 32 and 34. In some embodiments, the human subject is within an age group (in years) of 37 and 99, of 37 and 94, of 37 and 89, of 37 and 84, of 37 and 79, of 37 and 74, of 37 and 69, of 37 and 64, of 37 and 59, of 37 and 54, of 37 and 49, of 37 and 44, or of 37 and 39. In some embodiments, the human subject is within an age group (in years) of 42 and 99, of 42 and 94, of 42 and 89, of 42 and 84, of 42 and 79, of 42 and 74, of 42 and 69, of 42 and 64, of 42 and 59, of 42 and 54, of 42 and 49, or of 42 and 44. In some embodiments, the human subject is within an age group (in years) of 47 and 99, of 47 and 94, of 47 and 89, of 47 and 84, of 47 and 79, of 47 and 74, of 47 and 69, of 47 and 64, of 47 and 59, of 47 and 54, or of 47 and 49. In some embodiments, the human subject is within an age group (in years) of 52 and 99, of 52 and 94, of 52 and 89, of 52 and 84, of 52 and 79, of 52 and 74, of 52 and 69, of 52 and 64, of 52 and 59, or of 52 and 54. In some embodiments, the human subject is within an age group (in years) of 57 and 99, of 57 and 94, of 57 and 89, of 57 and 84, of 57 and 79, of 57 and 74, of 57 and 69, of 57 and 64, or of 57 and 59. In some embodiments, the human subject is within an age group (in years) of 62 and 99, of 62 and 94, of 62 and 89, of 62 and 84, of 62 and 79, of 62 and 74, of 62 and 69, or of 62 and 64. In some embodiments, the human subject is within an age group (in years) of 67 and 99, of 67 and 94, of 67 and 89, of 67 and 84, of 67 and 79, of 67 and 74, or of 67 and 69. In some embodiments, the human subject is within an age group (in years) of 72 and 99, of 72 and 94, of 72 and 89, of 72 and 84, of 72 and 79, or of 72 and 74. In some embodiments, the human subject is within an age group (in years) of 77 and 99, of 77 and 94, of 77 and 89, of 77 and 84, or of 77 and 79. In some embodiments, the human subject is within an age group (in years) of 82 and 99, of 82 and 94, of 82 and 89, or of 82 and 84. In some embodiments, the human subject is within an age group (in years) of 87 and 99, of 87 and 94, or of 87 and 89. In some embodiments, the human subject is within an age group (in years) of 92 and 99, or of 92 and 94.

In humans, the cornea is comprised of five layers, in order from the outside (body surface), of corneal epithelium, Bowman's membrane (external boundary), Lamina propria, Descemet's membrane (internal boundary), and corneal endothelium. Unless specifically noted otherwise, parts other than epithelium and endothelium may be collectively called corneal stroma. The corneal endothelium is composed of a single layer of cells located on the posterior surface of the cornea, facing the anterior chamber. The endothelium governs fluid and nutrient transport across the posterior surface of the cornea in a pump-and-barrier function and maintains the cornea in an optimized state required for optical transparency and optimal vision.

Unlike the epithelium, which has a self-renewing capacity, the endothelium is not capable of regenerating. Corneal endothelial disease may occur, for example, when endothelial cells degrade and/or are lost due to conditions such as bullous keratopathy, Fuchs' dystrophy, congenital corneal dystrophies or ocular surgical trauma. Corneal endothelial disease leads to symptoms that impact vision, including blurred vision, vision loss, corneal hydration, increased glare or discomfort, or severe ocular pain.

The methods of the present disclosure may treat or prevent corneal endothelial disease. Non-limiting examples of corneal endothelial diseases include bullous keratopathy, corneal endothelial dystrophies (e.g., cornea guttata, Fuchs endothelial corneal dystrophy, posterior polymorphous corneal dystrophy, iridocorneal endothelial syndrome, and congenital hereditary corneal endothelial dystrophy), viral diseases (e.g., cytomegalovirus endotheliitis and herpetic endotheliitis), exfoliation syndrome, and corneal endothelial graft rejection; as well as inflammation or physical damage associated with external factors, such as keratouveitis, interstitial keratitis, corneal endotheliitis, corneal endothelial cell loss after corneal transplantation, corneal injury after intraocular surgery (e.g., cataract surgery, vitreous surgery, glaucoma surgery), corneal injury induced by glaucomatous attack, corneal injury caused by long-term contact lens use, corneal trauma, corneal edema, and intrapartum corneal trauma. In certain embodiments, the therapeutics methods disclosed herein treat Fuchs endothelial corneal dystrophy.

Corneal endothelial diseases include those having different grades, such as corneal endothelial disorder Grade 3 (e.g., CEC density below 500 cells/mm2 but no corneal edema) or corneal endothelial disorder Grade 4 (e.g., bullous keratopathy). This grade system was previously established based upon the severity of the corneal endothelial disorders (e.g., see Japanese Journal of Ophthalmology 118:81-83, 2014).

In some embodiments, the corneal endothelial disease is a bullous keratopathy, a corneal edema (e.g., Corneal Edema Secondary to Corneal Endothelial Dysfunction), a corneal leukoma, a corneal endothelial inflammation, or a corneal dystrophy.

In some embodiments, the disease is a bullous keratopathy. In some embodiments, the corneal endothelial disease is Fuchs endothelial corneal dystrophy, PEX-BK (pseudoexfoliation bullous keratopathy; bullous keratopathy involving pseudoexfoliation syndrome), post-laser iridotomy bullous keratopathy, post-cataract surgery bullous keratopathy (including pseudophakic or aphakic bullous keratopathy), post-glaucoma surgery bullous keratopathy, intraocular surgery-related bullous keratopathy, post-trauma bullous keratopathy, bullous keratopathy of an unknown cause after multiple surgeries, post-corneal transplantation graft failure, congenital corneal endothelial dystrophy, or congenital anterior chamber angle hypoplasia syndrome.

In some embodiments, the corneal endothelial disease is one characterized by low endothelial cell count. In some embodiments, the corneal endothelial disease is one characterized by visual impairment, blurred vision, eye discomfort, eye pain, or corneal edema. In some embodiments, the disease is a corneal edema (e.g., Corneal Edema Secondary to Corneal Endothelial Dysfunction).

A CEC composition disclosed herein may be administered in conjunction with an additional agent. The additional agent may be co-formulated with the CECs into a single composition, or may be administered in as a separate agent but as a combination therapy. Agents that are generally used in ophthalmic therapy (e.g., steroid agent, antibiotic agent, or NSAID) may be used. Such an addition agent may be administered together with the cell composition or provided in a separately administered form. In various embodiments, any of such additional agents (e.g., steroid agent, antibiotic agent, or NSAID) or combinations thereof may also be administered post-administration of the CEC composition in any suitable administration route (e.g., as a topical eye cream). In various embodiments, the any one of the additional agents or combinations thereof are administered for at least about 1 day, at least about 3 days, at least about 7 days, at least about 14 days, at least about 21 days, at least about 27 days, at least about 1 month, at least about 3 months, at least about 6 months, at least about 9 months, or at least about 12 months post-administration of the CEC composition. In various embodiments, the any one of the additional agents or combinations thereof are administered for up to about 1 day, up to about 3 days, up to about 7 days, up to about 14 days, up to about 21 days, up to about 27 days, up to about 1 month, up to about 3 months, up to about 6 months, up to about 9 months, or up to about 12 months post-administration of the CEC composition. Examples of steroid agents include, without limitation, methylprednisolone, betamethasone, fluorometholone, dexamethasone, or prednisolone. An antibiotic agent may be flomoxef sodium, cefcapene pivoxil hydrochloride, or gatifloxacin. Examples of NSAIDs include indomethacin, planoprofen, diclofenac sodium, bromfenac sodium hydrate, or nepafenac.

Various delivery systems are known, and such systems can be used to administer the CEC composition to a suitable site (e.g., ocular anterior chamber). A typical dosage form of the cellular composition is injected into the anterior chamber.

In some embodiments, the CEC composition can be prepared as a pharmaceutical composition adapted to administration to humans in accordance with a known method. Such a composition can be administered by injection or infusion. When a composition is to be administered by injection, the composition can be distributed by using an injection bottle containing a pharmaceutically acceptable carrier, such as a cell infusion solution, aseptic agent-grade water or saline.

Exemplary doses are described in the Example.

In various embodiments, references to the methods disclosed herein (e.g., methods of treating or preventing a corneal endothelial disease disclosed herein) may be interpreted as references to pharmaceutical compositions and medicaments of the present disclosure for use in those methods.

### III. Corneal Endothelial Cell Compositions

The CEC compositions for use in the methods of the invention comprise human CECs, including human allogeneic CECs. In some embodiments, the human CECs are cultured (e.g., expanded, differentiated, and/or matured) prior to administration. In some embodiments, the CEC compositions disclosed herein are stored prior to administration (e.g., the CEC compositions are not fresh or freshly prepared).

In various embodiments, the CECs of the present disclosure are functional mature differentiated CECs. In various embodiments, the CECs are capable of eliciting a human corneal endothelial functional property when administered into an anterior chamber of a human eye. In various embodiments, a functional mature differentiated CEC of the present disclosure refers to a mature and differentiated CEC having a corneal endothelial functional property. In various embodiments, a corneal endothelial functional property refers to a functional property that a mature differentiated cornea or corneal cell has in a normal state (e.g., in the state of a native corneal endothelial cell). In various embodiments, a corneal endothelial functional property of a cell can be confirmed from, e.g., the cell forming a small hexagonal cobblestone shape and using an energy metabolism system by mitochondrial function. In various embodiments, a corneal endothelial functional property can be determined by a surrogate marker as an indicator. In some embodiments, determination can be made with any one of or any combination of surrogate markers selected from (1) retention of endothelial pumping/barrier functions (including claudin expression), (2) adhesion/attachment to a specific laminin, (3) secreted cytokine profile, (4) produced micro RNA (miRNA) profile, (5) produced metabolite profile, (6) saturated cell density upon in vitro culture, and (7) spatial size and distribution of cells obtained in culturing. In various embodiments, retention of endothelial pumping/barrier functions can be determined using a pumping function measuring method or a barrier function measuring method commonly used for corneal endothelia. Examples include techniques described in Wigham C, Hodson S.: Current Eye Research, 1, 37-41, 1981; Hodson S, Wigham C.: J Physiol., 342:409-419, 1983; Hatou S., Yamada M., Akune Y., Mochizuki H., Shiraishi A., Joko T., Nishida T., Tsubota K.: Investigative Ophthalmology & Visual Science, 51, 3935-3942, 2010. Claudin expression can be confirmed by using a known approach in the art such as an immunological approach. In various embodiments, adhesion/attachment to a specific laminin can be determined via measurement of adhesion to laminin 511 (composite of alpha5 chain, beta1 chain, and gamma1 chain), laminin 521 (composite of alpha5 chain, beta2 chain, and gamma1 chain), or a functional fragment thereof (e.g., laminin 511-E8 fragment) and/or increase in integrin (e.g., alpha3beta1, alpha6beta1 or the like) expression. Alpha5 chain (LAMA5) is a subunit of a protein (laminin) of a cell adhesion molecule in an extracellular matrix, and is called LAMA5, KIAA1907, or the like. For human LAMA5, the sequences of the gene and protein can be found through NCBI registration numbers NM_005560 and NP_005551, respectively. Beta1 chain (LAMB1) is a subunit of a protein (laminin) of a cell adhesion molecule in an extracellular matrix, and is called LAMB1, CLM, LIS5, or the like. For human LAMB1, the sequences of the gene and protein can be found through NCBI registration numbers NM_002291 and NP_002282, respectively. Beta2 chain (LAMB2) (laminin S) is a subunit of a protein (laminin) of a cell adhesion molecule in an extracellular matrix, and is called LAMB2, LAMS, NPHS5, or the like. For human LAMB2, the sequences of the gene and protein can be found through NCBI registration numbers NM_002292 and NP_002283, respectively. Gamma1 chain (LAMC1) is a subunit of a protein (laminin) of a cell adhesion molecule in an extracellular matrix, and is called LAMC1, LAMB2, or the like. For human LAMC1, the sequences of the gene and protein can be found through NCBI registration numbers NM_002293 and NP_002284, respectively. In various embodiments, secreted cytokine profiles can be determined by measuring the production level of cytokines in biological samples (e.g., serum, or anterior aqueous humor of the eye). Such cytokines include, without limitation to, RANTES, PDGF-BB, IP-10, MIP-1b, VEGF, EOTAXIN, IL-1ra, IL-6, IL-7, IL-8, IL-0, IL-10, IL-12 (p70), IL-13, IL-17, FGFbasic, G-CSF, GM-CSI, IFN-gamma, MCP-1, MIP-1a, TNF-alpha, and the like. Analysis can be performed using a cytokine measuring kit and analysis system such as Bio-Plex for integrated analysis of cytokines.

In various embodiments, the produced microRNA (miRNA) profile can be determined using analysis methods known in the art. For example, Toray's “3D-Gene” human miRNA oligochip (miRBase version 17) can be used. In various embodiments, the produced metabolite profile can be determined using analysis methods known in the art. For example, a metabolic extract of an intracellular metabolite can be prepared from a cell culture (e.g., CEC culture) sample having methanol and an internal standard reagent such as Internal Standard Solution (Human Metabolome Technologies; HMT, Inc., Tsuruoka, Japan). Capillary electrophoresis-mass spectrometry (CE-MS) analysis can be preformed to analyze the metabolite. Metabolome profile can be measured according to a method developed by Soga, et al. (Soga, D. et al., T. Soga, et al., Anal. Chem. 2002; 74:2233-2239 Anal. Chem. 2000; 72:1236-1241; T. Soga, et al., J. Proteome Res. 2003; 2:488-494) with automatic integration software (MasterHands, Keio University, Tsuruoka, Japan (M. Sugimoto, et al., Metabolomics, 2009; 6:78-95) and analyzed with MassHunter Quantitative Analysis (Agilent Technologies, Santa Clara, CA, USA). Examples of metabolites include any products related to a product of energy metabolism system in a mitochondrial system, a glutathione metabolic system product, a methionine metabolic cycle product, a lipid metabolite, a pentose phosphate pathway product, a tricarboxylic acid (TCA) cycle metabolite, or a glycolytic system metabolite, such as succinic acid (succinate), Pro, Gly, glycerol 3-phosphate, Glu, lactic acid (lactate), arginosuccinic acid (arginosuccinate), xanthine, N-carbamoyl aspartic acid (N-carbamoyl aspartate), isocitric acid (isocitrate), cis-aconitic acid (cis-aconitate), citric acid (citrate), Ala, 3-phosphoglyceric acid (3-phosphoglycerate), hydroxyproline, malic acid (malate), uric acid (urate), betaine, folic acid (folate), Gln, 2-oxoisovaleric acid (2-oxoisovalerate), pyruvic acid (pyruvate), Ser, hypoxanthine, Asn, Trp, Lys, choline, Tyr, urea, Phe, Met, carnosine, Asp, ornithine, Arg, creatine, 2-hydroxy glutaminic acid (2-hydroxy glutamate), beta-Ala, citrulline, Thr, Ile, Leu, Val, creatinine, His, or N,N-dimethyl glycine. In various embodiments, the saturated cell density during in vitro culture can be determined by measuring the cell density under appropriate culture conditions described herein. Cell density may be measured in parallel with the cell size. In various embodiments, cell density and/or cell size may be determined using photo-taking phase contrast microscope images taken with a suitable image capturing system, e.g., a BZ X-700 Microscope (Keyence, Osaka, Japan) with an inverted microscope system (CKX41, Olympus, Tokyo, Japan). Cell density can be quantified with suitable cell counting software (e.g., BZ-H3C Hybrid cell count software (Keyence)). In various embodiments, the spatial size and distribution of cells in culture (e.g., in culture dish) can also be determined using the measurements and images of the cells.

In some embodiments, the cultured corneal endothelial cells (CECs) may have at least one, at least two, or all of the following characteristics (e.g., characteristics similar to CECs found in vivo).
(1) The cells may have a monolayer structure.
(2) The cell density of the cells may be about 10 to-about 10,000 cells/mm2.
(3) The visual flat plane shape of the cells may be approximately hexagonal.
(4) In the cell layer, cells may be regularly aligned.
(5) the cells may express markers characteristic of CECs found in vivo. For example, the CECs have a cell surface expression of a marker selected from the group consisting of CD166 positive, CD44 negative to CD44 weakly positive, CD24 negative to weakly positive, CD44 negative to weakly positive, CD105 negative to weakly positive, CD26 negative to weakly positive, CD200 negative to weakly positive, and CD90 negative to weakly positive phenotypes. In some embodiments, the CECs have a cell surface expression at the end of P4 selected from the group consisting of sodium-potassium ATPase, ZO-1, VDAC3, SLC4A4, CLCN3, COL4A2, COL8A1, COL8A2, CDH2, CD98, CD166, CD340, Integrin a3B1, CD56, Prdx-6, CD248, SLC4A11, and CYYR1.

In various embodiments, a CEC of the present disclosure (e.g., a CEC of a CEC composition disclosed herein, such as a non-fresh CEC composition disclosed herein) expresses a phenotype (e.g., a cell surface marker phenotype) disclosed herein. As used herein, “cell surface marker” refers to any biological material expressed on a cell surface. This may also be referred to as a cell surface antigen, surface antigen, or surface marker. A cell surface marker can be identified as an antigen binding to a monoclonal antibody. Cell surface markers referred to as CD (cluster of differentiation) markers or CD antigens in the art are also encompassed. In various embodiments, a CEC (e.g., a cultured human CEC) of the present disclosure has a marker phenotype disclosed herein. In some embodiments, a CEC (e.g., a cultured human CEC) of the present disclosure is CD166 positive, CD105 negative to weakly positive, and CD44 negative to weakly positive. In some embodiments, a CEC (e.g., a cultured human CEC) of the present disclosure is CD24 negative. In some embodiments, a CEC (e.g., a cultured human CEC) of the present disclosure is CD26 negative. In some embodiments, a CEC (e.g., a cultured human CEC) of the present disclosure is CD166 positive, CD105 negative to weakly positive, CD24 negative, and CD44 negative to weakly positive. In some embodiments, a CEC (e.g., a cultured human CEC) of the present disclosure is CD166 positive, CD105 negative to weakly positive, CD26 negative, and CD44 negative to weakly positive. In some embodiments, a CEC (e.g., a cultured human CEC) of the present disclosure is CD166 positive, CD105 negative, CD24 negative, and CD44 negative. In some embodiments, a CEC (e.g., a cultured human CEC) of the present disclosure is CD166 positive, CD105 negative, CD26 negative, and CD44 negative. In some embodiments, a CEC (e.g., a cultured human CEC) of the present disclosure is CD166 positive, CD105 negative, CD26 negative, CD24 negative, and CD44 negative. In some embodiments, a CEC (e.g., a cultured human CEC) of the present disclosure is CD90 negative to weakly positive, CD133 negative, CD26 negative, LGR5 negative, SSEA3 negative, MHC1 weakly positive, MHC2 negative, PDL1 positive, ZO1 positive, or Na+/K+ ATPase positive. In some embodiments, a CEC (e.g., a cultured human CEC) of the present disclosure is CD90 negative to weakly positive.

In various embodiments, a CEC of the present disclosure (e.g., a CEC of a CEC composition disclosed herein, such as a non-fresh CEC composition disclosed herein) is capable of adhering to surrounding cells. In various embodiments, the CEC expresses tight junction protein ZO-1. In various embodiments, a CEC of the present disclosure expresses Na+/K+ ATPase (ATP-dependent sodium-potassium pump). In various embodiments, a CEC of the present disclosure expresses ZO-1 and Na+/K+ ATPase.

In various embodiments, a CEC of the present disclosure is CD90 negative to weakly positive, CD133 negative, CD26 negative, LGR5 negative, SSEA3 negative, MHC1 weakly positive, MHC2 negative, PDL1 positive, ZO-1 positive, or Na+/K+ ATPase positive. In some embodiments, the CEC is CD200 negative. In various embodiments, a CEC of the present disclosure is CD105 negative to weak positive, CD24 negative, CD26 negative, LGR5 negative, SSEA3 negative, MHC1 weak positive, MHC2 negative, ZO-1 positive, Na⁺/K⁺ ATPase positive.

In various embodiments, a CEC of the present disclosure expresses at least one of sodium-potassium ATPase, ZO-1, VDAC3, SLC4A4, CLCN3, COL4A2, COL8A1, COL8A2, CDH2, CD98, CD166, CD340, Integrin a3B1, CD56, Prdx-6, CD248, SLC4A11, and CYYR1.

The cell marker phenotype (e.g., the cluster of differentiation (CD) marker phenotype) may be identified via common techniques and methods in the art, such as flow cytometry analysis. Cell immunophenotyping via flow cytometry is well known in the art. The characterization of positive and negative (or weekly positive) marker profiles through gating has also been well known in the art. Gating is a fundamental aspect of flow cytometry and a skilled person understands how to gate and analyze data when immunophenotyping cells (e.g., CECs disclosed herein). For example, an initial gating (forward scatter vs. side scatter) can be performed to exclude debris. Singlet population and viability (live/dead) cells can then be determined. For the markers (e.g., the CD markers disclosed herein), the left side of the interval gate for each marker in the multicolor flow cytometry analysis can be set based on the pre-qualification Fluoresce Minus One (FMO) control for the marker. The upper limit of the FMO control's fluorescence distribution can be used to set the negative gating. Cells with fluorescence well above the FMO-defined boundary and form a clearly separate population may be considered positive. Cells in the fully stained sample that fall just above the FMO-defined negative boundary but do not form a clearly separate population may be considered weakly positive.

In various embodiments, at least 50%, at least 55%, at least 60%, at least 65%, at least 70%, at least 75%, at least 80%, at least 85%, at least 90%, at least 95%, or at least 99% of the human CECs of a composition of the present disclosure (e.g., a non-fresh CEC composition disclosed herein) have a marker phenotype disclosed herein. In various embodiments, about 50%, about 55%, about 60%, about 65%, about 70%, about 75%, about 80%, about 85%, about 90%, about 95%, or about 99% of the CECs of a composition of the present disclosure (e.g., a non-fresh CEC composition disclosed herein) have a marker phenotype disclosed herein.

In various embodiments, at least 70%, at least 75%, at least 80%, at least 85%, at least 90%, at least 95%, at least 97%, at least 98%, or at least 99% of the human CECs of a composition of the present disclosure (e.g., a non-fresh CEC composition disclosed herein) are CD166 positive, CD105 negative to weakly positive, CD26 negative, and CD44 negative to weakly positive. In various embodiments, at least 70%, at least 75%, at least 80%, at least 85%, at least 90%, at least 95%, at least 97%, at least 98%, or at least 99% of the human CECs of a composition of the present disclosure (e.g., a non-fresh CEC composition disclosed herein) are CD166 positive, CD105 negative, CD26 negative, and CD44 negative. In various embodiments, at least 70%, at least 75%, at least 80%, at least 85%, at least 90%, at least 95%, at least 97%, at least 98%, or at least 99% of the human CECs of a composition of the present disclosure (e.g., a non-fresh CEC composition disclosed herein) are CD166 positive, CD105 negative to weakly positive, CD24 negative, and CD44 negative to weakly positive. In various embodiments, at least 70%, at least 75%, at least 80%, at least 85%, at least 90%, at least 95%, at least 97%, at least 98%, or at least 99% of the human CECs of a composition of the present disclosure (e.g., a non-fresh CEC composition disclosed herein) are CD166 positive, CD105 negative, CD24 negative, and CD44 negative. In various embodiments, at least 70%, at least 75%, at least 80%, at least 85%, at least 90%, at least 95%, at least 97%, at least 98%, or at least 99% of the human CECs of a composition of the present disclosure (e.g., a non-fresh CEC composition disclosed herein) are CD166 positive, CD105 negative to weakly positive, CD26 negative, CD24 negative, and CD44 negative to weakly positive. In various embodiments, at least 70%, at least 75%, at least 80%, at least 85%, at least 90%, at least 95%, at least 97%, at least 98%, or at least 99% of the human CECs of a composition of the present disclosure (e.g., a non-fresh CEC composition disclosed herein) are CD166 positive, CD105 negative, CD26 negative, CD24 negative, and CD44 negative.

In various embodiments, a CEC composition comprising human CECs disclosed herein has one or more of the following characteristics:
(1) when assessed by culture supernatant enzyme-linked immunoassay assay (ELISA):
   TIMP-1: 500 ng/mL or less
   IL-8: 500 pg/mL or less
   PDGF-BB: 30 pg/mL or greater
   MCP-1: 3000 pg/mL or less
(2) when assessed by fluorescence-activated cell sorting (FACS):
   CD166 positive=95% or greater
   CD133 positive=5% or less
   CD105 low positive=95% or greater
   CD44 low positive=80% or greater
   CD44 high positive=5% or less
   CD24 positive=5% or less
   CD26 positive=5% or less
   CD200 positive=5% or less
(3) barrier function (ZO-1) positive
(4) pumping function (Na+/K+ATPase) positive
(5) CD44 strongly positive cell<5%, CD26 positive cell<5 %, or CD24 positive cell<5%
(6) karyotype abnormality negative.

In various embodiments, a composition of the present disclosure comprises neltependocel. As used herein, “neltependocel” refers to cultured, allogeneic human CECs. In preferred embodiments, at least 75%, at least 80%, at least 85%, at least 90%, at least 95%, at least 97%, at least 98%, or at least 99% of the CECs of neltependocel are CD166 positive, CD105 negative to weakly positive, CD24 negative, and CD44 negative to weakly positive. In further preferred embodiments, at least 75%, at least 80%, at least 85%, at least 90%, at least 95%, at least 97%, at least 98%, or at least 99% of the CECs of neltependocel are CD166 positive, CD105 negative, CD24 negative, and CD44 negative.

Measurement and quantification of polynucleotide or polypeptide expression can be accomplished by suitable methods known in the art, including, for example, northern blot, dot blot, PCR, immunohistochemistry methods such as ELISA, radioimmunoassay (RIA), fluorescent antibody, luminescence immunoassay (LIA), immunoprecipitation (IP), radial immunodiffusion (RID), turbidimetric immunoassay (TIA), western blot, immunohistochemical staining, and flow cytometry methods.

In various embodiments, a CEC of the present disclosure (e.g., a CEC of a CEC composition disclosed herein, such as a non-fresh CEC composition) has a hexagonal morphology as detected by microscopy.

In various embodiments, a CEC of the present disclosure (e.g., a CEC of a CEC composition disclosed herein, such as a non-fresh CEC composition) has a cytokine production profile of at least one of high PDGF-BB production, low IL-8 production, low MCP-1 production, high TNFα production, high IFNγ production, high IL-IR antagonist production, or low VEGF production. As used herein, “high production” and “low production” are relative and determined as being high or low compared to a generally observed level for each cytokine or the like. For instance, it is typically preferable that PDGF-BB is about 30 μg/ml or greater and IL-8 is about 500 μg/ml. Further, MCP-1 is preferably about 3000 μg/ml or less. TNFα is preferably about 10 μg/ml or greater, and IFNγ is preferably about 30 μg/ml or greater. IL-1R antagonists are preferably about 40 μg/ml or greater, and VEGF is preferably about 200-500 μg/ml or less.

In various embodiments, a CEC of the present disclosure (e.g., a CEC of a CEC composition disclosed herein, such as a non-fresh CEC composition) does not have a karyotype abnormality (e.g., aneuploidy which may be induced in cell culture due to cell division). For humans, karyotype abnormalities can be measured in accordance with the Standard International System for Human Cytogenetic Nomenclature (ISCN) (1995) and definitions thereof.

Having such morphological characteristics, the composition herein may have functions similar to those of the corneal endothelial cells in living organisms.

In various embodiments, the CECs of the present disclosure are sourced from a donor cornea. In various embodiments, the CECs are sourced from CECs of a donor cornea (e.g., are corneal endothelial tissue derived cells). In various embodiments, the CECs are not sourced from CECs of a donor cornea. In various embodiments, the CECs are differentiated from pluripotent stem cells (e.g., iPSCs), mesenchymal stem cells, corneal endothelial progenitor cells (e.g., precursor cells harvested from a corneal endothelium), cells harvested from a corneal endothelium, or corneal endothelial precursor cells or corneal endothelial-like cells produced via a direct programing method (e.g., corneal endothelial progenitor cells or corneal endothelial-like cells differentiated from stem cells, e.g., iPSCs). In various embodiments, the CECs are differentiated from pluripotent stem cells (e.g., iPSCs).

In various embodiments, about 1×10⁴ CECs, about 2.5×10⁴ CECs, about 5×10⁴ CECs, about 7.5×10⁴ CECs, about 1×10⁵ CECs, about 2.5×10⁵ CECs, about 5×10⁵ CECs, about 7.5×105 CECs, about 1×10⁶ CECs, about 2.5×10⁶ CECs, about 5×10⁶ CECs, or about 7.5×10⁶ CECs of the present disclosure are administered to the subject, or a composition of the present disclosure comprises about 1×10⁴ CECs, about 2.5×10⁴ CECs, about 5×10⁴ CECs, about 7.5×10⁴ CECs, about 1×10⁵ CECs, about 2.5×10⁵ CECs, about 5×10⁵ CECs, about 7.5×10⁵ CECs, about 1×10⁶ CECs, about 2.5×10⁶ CECs, about 5×10⁶ CECs, or about 7.5×10⁶ CECs. In various embodiments, about 2.5×10⁵ CECs, about 5×10⁵ CECs, about 7.5×10⁵ CECs, about 1×10⁶ CECs, or about 2.5×10⁶ CECs of the present disclosure are administered to the subject, or a composition of the present disclosure comprises about 2.5×10⁵ CECs, about 5×10⁵ CECs, about 7.5×10⁵ CECs, about 1×10⁶ CECs, or about 2.5×10⁶ CECs.

In various embodiments, the CECs of a composition disclosed herein achieve a mean cell density of at least about 1500 cells/mm² or higher, at least about 1600 cells/mm² or higher, at least about 1700 cells/mm² or higher, at least about 1800 cells/mm² or higher, at least about 1900 cells/mm² or higher, or at least about 2000 cells/mm² or higher after administration to a human subject (e.g., to an eye of the subject) and integration into a human corneal endothelial surface.

In various embodiments, the CECs of a composition disclosed herein are in a suspension form. As use herein, “suspension” refers to a non-adherent, re-suspendable preparation of cells in a liquid vehicle in which the cells remain capable of being maintained and/or redistributed throughout the liquid phase with minimal agitation. A “suspension” includes preparations in which the cells are present as single cells and/or as cell clusters, microaggregates, or larger aggregates, and encompasses states in which the cells are momentarily dispersed throughout the liquid or have settled or precipitated under gravity during storage but can be readily redispersed by gentle mixing. Without limitation, a “suspension” thus includes: (i) cells uniformly dispersed in the medium; (ii) cells that have partially or fully settled to the bottom of the container over time but are not substantially adherent and can be redispersed by inversion, gentle shaking, or pipetting; and (iii) cells present as single cells, loose clusters, or compact aggregates that remain non-adherent to the container surface and maintain the capacity to be redistributed throughout the medium. A preparation is not a “suspension” if the cells are substantially adherent to the container surface such that they cannot be redistributed throughout the liquid phase without enzymatic treatment or forceful mechanical detachment. In various embodiments, the CECs of a composition disclosed herein are not substantially adherent to a surface of a container (e.g., a vial) of the composition comprising the CECs. In various embodiments, “substantially adherent to a surface of a container” refers to at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the CECs adhering to the surface of the container. In various embodiments, the CECs are in a suspension form or are not substantially adherent to a surface of a container (e.g., a vial) of a composition comprising the CECs. In various embodiments, the CECs are in a suspension form or are not substantially adherent to a surface of a container (e.g., a vial) of a composition comprising the CECs, while the CECs maintain a cell adhesion characteristic disclosed herein (e.g., capability of a CEC to adhere to a substrate or another CEC).

The CEC compositions provided herein may be formulated with or without a ROCK inhibitor. Previously, ROCK inhibition was considered necessary to promote adhesion of corneal endothelial cell in cell culture (see, e.g. U.S. Pat. No. 11,633,404B2). Preferably, a pharmaceutical composition used in the methods disclosed herein comprises CECs and a ROCK inhibitor, such as Y-27632.

Examples of ROCK inhibitors used as a combined agent include compounds disclosed in U.S. Pat. No. 4,678,783, Japanese Patent No. 3421217, WO 95/28387, WO 99/20620, WO 99/61403, WO 02/076976, WO 02/076977, WO 2002/083175, WO 02/100833, WO 03/059913, WO 03/062227, WO 2004/009555, WO 2004/022541, WO 2004/108724, WO 2005/003101, WO 2005/039564, WO 2005/034866, WO 2005/037197, WO 2005/037198, WO 2005/035501, WO 2005/035503, WO 2005/035506, WO 2005/080394, WO 2005/103050, WO 2006/057270, WO 2007/026664. Such compounds can be manufactured by the method described in each disclosed document. Examples thereof include 1-(5-isoquinolinesulfonyl) homopiperazine or a salt thereof (e.g., fasudil or fasudil hydrochloride), (+)-trans-4-(1-aminoethyl)-1-(4-pyridylcarbamoyl)cyclohexanecarboxamide or a salt thereof (e.g., Y-27632 ((R)-(+)-trans-(4-pyridyl)-4-(1-aminoethyl)-cyclohexanecarboxamide dihydrochloride monohydrate), and the like), and preferably comprising Y-27632. In various embodiments, the ROCK inhibitor is Y-27632.

In various embodiments, a ROCK inhibitor, such as Y-27632, is administered to the subject, or a composition of the present disclosure comprises a ROCK inhibitor, such as Y-27632, at a concentration of at least 10 UM, at least 15 μM, at least 20 μM, at least 25 μM, at least 30 μM, at least 35 μM, at least 40 μM, at least 45 μM, at least 50 μM, at least 55 μM, at least 60 μM, at least 65 μM, at least 70 μM, at least 75 μM, at least 80 μM, at least 85 μM, at least 90 μM, at least 95 μM, at least 100 μM, at least 105 M, at least 110 μM, at least 115 μM, or at least 120 μM. In various embodiments, a ROCK inhibitor, such as Y-27632, is administered to the subject, or a composition of the present disclosure comprises a ROCK inhibitor, such as Y-27632, at a concentration of about 10 μM, about 15 μM, about 20 μM, about 25 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM, about 100 μM, about 105 μM, about 110 μM, about 115 μM, or about 120 μM. In various embodiments, a ROCK inhibitor, such as Y-27632, is administered to the subject, or a composition of the present disclosure comprises a ROCK inhibitor, such as Y-27632, at a concentration of about 100 μM.

In various embodiments, a composition of the present disclosure is AURN001. As used herein, “AURN001” refers to a composition comprised of neltependocel and Y-27632. In preferred embodiments, AURN001 comprises about 85 μM, about 90 μM, about 95 μM, about 100 μM, about 105 μM, about 110 μM, about 115 μM, or about 120 μM of Y-27632. In further preferred embodiments, AURN001 comprises about 100 μM of Y-27632. In preferred embodiments, AURN001 comprises neltependocel at about 2.5×10⁵ cells, about 5×10⁵ cells, about 7.5×10⁵ cells, about 1×10⁶ cells, about 2.5×10⁶ cells, or about 5×10⁶ cells. In further preferred embodiments, AURN001 comprises neltependocel at about 1×10⁶ cells. In some embodiments, AURN001 comprises neltependocel at about 1×10⁶ cells and about 100 μM of Y-27632.

Further provided are pharmaceutical compositions including the CECs and a pharmaceutically acceptable carrier or excipient. As used herein, “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained. As used herein “carrier” refers to a culture, infusion vehicle, irrigating solution, diluent, adjuvant, excipient or vehicle administered in conjunction with a medicament, such as a cellular composition provided herein.

Such a composition contains a therapeutically effective amount of cellular agent together with a suitable amount of carrier, such that the composition is provided in a form suitable for administration to a patient. In some embodiments, the pharmaceutically acceptable carrier is a cell infusion vehicle. The cell infusion vehicle can be any solution in which a cell can be maintained.

The composition can be prepared as a pharmaceutical composition adapted to administration to humans in accordance with a known method. Such a composition can be administered by injection or infusion. When a composition is to be administered by injection, the composition can be distributed by using an injection bottle containing cell infusion solution, aseptic agent-grade water or saline.

In various embodiments, a CEC composition (e.g., comprising CECs and Y-27632) is formulated with one or more of the following: Human Serum Albumin (HSA), Dulbecco's Modified Eagle Medium (DMEM), glucose, and/or HEPES. In various embodiments, a CEC composition (e.g., comprising CECs and Y-27632) is formulated with the following: Human Serum Albumin (HSA), Dulbecco's Modified Eagle Medium (DMEM), glucose, and HEPES. In various embodiments, a CEC composition (e.g., comprising CECs and Y-27632) is formulated with Human Serum Albumin (HSA) and Dulbecco's Modified Eagle Medium (DMEM). In various embodiments, a CEC composition (e.g., comprising CECs and Y-27632) is formulated with Dulbecco's Modified Eagle Medium (DMEM). In various embodiments, a CEC composition (e.g., comprising CECs and Y-27632) is formulated with 2% HSA.

In some aspects, a composition disclosed herein (e.g., comprising CECs and a Rho-associated, coiled-coil containing protein kinase inhibitor (e.g., Y-27632)) is freshly prepared, formulated, and administered (i.e., a “fresh composition”). In other aspects, the composition is not freshly prepared, formulated, and administered (i.e., a “non-fresh composition”). As used herein, a “fresh composition” refers to a CEC composition wherein the CECs are subjected to any culturing and/or formulation as disclosed herein and the composition is administered (e.g., to a subject with a corneal endothelial disease), after any culturing and/or formulation, without an intervening storage period extending beyond routine short-term hold conditions used during processing and release. In certain embodiments, a fresh composition is administered within 3 hours of the culturing and/or formulation. A “non-fresh composition” refers to a CEC composition wherein the CECs are subjected to any culturing and/or formulation as disclosed herein and the composition is then stored for up to about 72 hours before administration (e.g., to a subject with a corneal endothelial disease). In various aspects, a non-fresh composition (e.g., comprising CECs and Y-27632) is stored at between 2° C. and 8° C. before administered to a patient. In various aspects, a non-fresh composition (e.g., comprising CECs and Y-27632) is refrigerated at between 2° C. and 8° C. before administered to a patient.

In some aspects, a non-fresh composition (e.g., comprising CECs and Y-27632) administered to a patient is prepared or formulated about 24 h, about 36 h, about 48 h, or about 72 h in advance. In some aspects, a non-fresh composition (e.g., comprising CECs and Y-27632) administered to a patient is prepared or formulated at most about 24 h, about 36 h, about 48 h, or about 72 h in advance. In some aspects, a non-fresh composition (e.g., comprising CECs and Y-27632) administered to a patient is prepared or formulated at least about 3 h, about 4 h, about 5 h, about 6 h, about 7 h, about 8 h, about 9 h, about 10 h, about 11 h, or about 12 h in advance, but at most about 24 h, about 36 h, about 48 h, or about 72 h in advance. In some aspects, a non-fresh composition (e.g., comprising CECs and Y-27632) administered to a patient is prepared or formulated about 3 h to about 72 h in advance. In some aspects, a non-fresh composition (e.g., comprising CECs and Y-27632) administered to a patient is prepared or formulated about 3 h to about 48 h in advance. In some aspects, a non-fresh composition (e.g., comprising CECs and Y-27632) administered to a patient is prepared or formulated at least about 3 h but at most about 24 h, about 36 h, about 48 h, or about 72 h in advance. In some aspects, a non-fresh composition (e.g., comprising CECs and Y-27632) administered to a patient is prepared or formulated at least about 4 h but at most about 24 h, about 36 h, about 48 h, or about 72 h in advance. In some aspects, a non-fresh composition (e.g., comprising CECs and Y-27632) administered to a patient is prepared or formulated at least about 5 h but at most about 24 h, about 36 h, about 48 h, or about 72 h in advance. In some aspects, a non-fresh composition (e.g., comprising CECs and Y-27632) administered to a patient is prepared or formulated at least about 6 h but at most about 24 h, about 36 h, about 48 h, or about 72 h in advance. In some aspects, a non-fresh composition (e.g., comprising CECs and Y-27632) administered to a patient is prepared or formulated at least about 9 h but at most about 24 h, about 36 h, about 48 h, or about 72 h in advance. In some aspects, a non-fresh composition (e.g., comprising CECs and Y-27632) administered to a patient is prepared or formulated at least about 12 h but at most about 24 h, about 36 h, about 48 h, or about 72 h in advance.

In various embodiments, CECs of a non-fresh composition disclosed herein maintain, after storage, a characteristic (e.g., cell surface marker phenotype) of the CECs disclosed herein. In various embodiments, a non-fresh composition disclosed herein maintains, after storage, at least 50%, at least 55%, at least 60%, at least 65%, at least 70%, at least 75%, at least 80%, at least 85%, or at least 90% viability of the CECs of the composition. In various embodiments, a non-fresh composition disclosed herein maintains, after storage, at least 70% viability of the CECs of the composition. In various embodiments, CECs of a non-fresh composition disclosed herein maintain, after storage, a capability of cell adhesion. Cell adhesion may be mediated through transmembrane adhesion molecules linked to the intracellular cytoskeleton, and in various embodiments refers to the capability of the CECs to adhere to a substrate (e.g., to the Descemet's membrane when used in a therapy for a corneal endothelial dysfunction). In various embodiments, cell adhesion may be represented by adhesions of the CECs (e.g., the cultured human CECs) to a substrate, such as collagen of a collagen coated cell culturing dish, and quantified by the number of attached cells. In various embodiments, cell adhesion may be represented by expression or production of ZO-1, actin, and/or vinculin of the CECs. Cell count, cell viability, and cell adhesion may be measured by methods known in the art, such as through flow cytometry, phase contrast images and actin fiber staining, CellTiter-Glo® luminescent cell viability assay (Promega), or an automated cell counter platform such as the NucleoCounter® NC-202™ counter using the Via2-Cassette™.

In some aspects, a composition described herein (e.g., comprising CECs and Y-27632) is injected in a subject's eye in an amount around 200, 210, 220, 230, 240, 250, 260, 270, 280, 290, 300, 310, 320, 330, 340, 350, 360, 370, 380, 390, or 400 μL, or more. In some aspects, a pharmaceutical composition described herein (e.g., comprising CECs and Y-27632) is injected in a subject's eye in an amount around 300 μL. In other aspects, around 9.6 μg of Y-27632 is delivered to a patient's eye together with neltependocel (e.g., around 1×10⁶ cells).

### Methods of Making CEC Compositions

Methods for harvesting and culturing CEC cells in suspension are generally known in the art (See, for example, Kinoshita, S., et al. (2018). New England Journal of Medicine, 378 (11), 995-1003).

Corneal endothelial cells (e.g., human CECs) may be collected by any conventional methods known in the art from the cornea of a suitable corneal donor. In brief, the CECs may be isolated by stripping Descemet's membrane, followed by enzyme treatment to remove the collagen matrix. These cells may undergo further analysis to confirm their biological characteristics and to verify criteria for therapeutic use. Markers for characterizing CECs and methods of identifying CECs are known in the art (See, e.g., Hamuro J, et al. Invest Ophthalmol Vis Sci. 2016 Aug. 1; 57 (10): 4385-92. doi: 10.1167/iovs.16-19771. PMID: 27564520; and Wongvisavavit, R., et al (2021). Regenerative medicine, 16 (09), 871-891, which are each hereby incorporated by reference).

In some embodiments, the CECs are from a human CEC primary cell line. Homogeneous corneal endothelial cells may be prepared using methods known in the art. For example, the Descemet's membrane and the endothelial cell layer of a corneal tissue may be detached from the corneal stroma, transferred into a culture vessel (e.g., a culture dish), and treated with an enzyme, such as collagenase A. In some embodiments, the CECs with Descemet's membrane and the endothelial cell layer are digested in a basal growth medium (e.g., OPTI-MEM® I Reduced Serum Media (Thermo Fisher Scientific, Inc., e.g., free of ammonium meta vanadate combined with fetal bovine serum (e.g., 8%)), which may be supplemented with additional components such as calcium chloride (e.g., 200 mg/L), chondroitin sulfate (e.g., 0.08%), and/or an antibiotic (e.g., gentamicin). The CECs may be digested at 37° C. for two to 24 hours. As a result, the corneal endothelial cells are detached from the Descemet's membrane. The corneal endothelial cells remaining in the Descemet's membrane can be further detached by mechanical methods, such as pipetting. This step may additionally include one or more washing steps (e.g., using the basal growth medium without an enzyme).

After removal of the Descemet's membrane, the corneal endothelial cells may then be cultivated in a suitable culture medium that permits growth of CECs (e.g., in an initial culture at passage 0). For example, in some embodiments, the CECs are cultured in a basal growth medium (e.g., OPTI-MEM® I Reduced Serum Media (Thermo Fisher Scientific, Inc., e.g., free of ammonium meta vanadate and combined with fetal bovine serum (e.g., 8%)). In some embodiments, the basal growth medium is further supplemented with an epidermal growth factor (EGF) and/or ascorbic acid (e.g., 20 μg/mL). In some embodiments, the basal growth medium further comprises a Rho-associated protein kinase (ROCK)-inhibitor, such as Y-27632. As a further example, commercially available DMEM (Dulbecco's Modified Eagle's Medium) appropriately supplemented with FBS (fetal bovine serum), b-FGF (basic-fibroblast growth factor) and antibiotics such as penicillin, streptomycin and the like can be used.

The temperature for cultivating corneal endothelial cells is not limited as long as the cells proliferate. For example, in some embodiments, the cells are cultured at a temperature of about 25° C. to about 45° C. or about 30° C. to about 40° C. In certain embodiments, the cells are cultured at a temperature of about 37° C. As a cultivation method, the cells may be cultured in a conventional incubator for cell culture under humidification in an environment of about 5-10% CO₂.

Additional objects, advantages, and novel features of the present invention will become apparent to one ordinarily skilled in the art upon examination of the following examples, which are not intended to be limiting.

Additionally, each of the various embodiments and aspects of the present invention as delineated hereinabove and as claimed in the claims section below finds experimental support in the following examples.

### EXAMPLES OF EMBODIMENTS OF THE PRESENT DISCLOSURE

Specific embodiments of the present disclosure include, but are not limited to, the following:

Embodiment 1. A method for treating a corneal endothelial disease in a human subject in need thereof, comprising administering an effective amount of a corneal endothelial cell (CEC) composition and an effective amount of a Rho-associated, coiled-coil containing protein kinase inhibitor to the human subject, wherein the composition comprises allogeneic human CECs, and wherein the corneal endothelial disease is a bullous keratopathy, a corneal edema, a corneal leukoma, a corneal endothelial inflammation, or a corneal dystrophy.

Embodiment 2. A method for treating or preventing a corneal endothelial disease in a human subject in need thereof, comprising administering an effective amount of a corneal endothelial cell (CEC) composition and an effective amount of a Rho-associated, coiled-coil containing protein kinase inhibitor to the human subject, wherein the CEC composition comprises allogeneic human CECs, and wherein the human subject has corneal edema secondary to corneal endothelial dysfunction.

Embodiment 3. The method of embodiment 2, wherein the human subject achieves >15-letter improvement (>3-line gain using the Early Treatment Diabetic Retinopathy Standard (ETDRS) visual acuity test) from baseline in a best corrected visual acuity (BCVA) at six months.

Embodiment 4. The method of embodiment 2 or 3, wherein the human subject has a change from baseline in BCVA and in central corneal thickness (CCT) at six months.

Embodiment 5. The method of embodiment 2, 3 or 4, wherein the human subject has no loss from baseline in BCVA of more than 15 letters.

Embodiment 6. The method of any one of embodiments 1-5, wherein the CEC composition comprises about 2.5×10⁵ CECs.

Embodiment 7. The method of any one of embodiments 1-5, wherein the CEC composition comprises about 5×10⁵ CECs.

Embodiment 8. The method of any one of embodiments 1-5, wherein the CEC composition comprises about 1×10⁶ CECs.

Embodiment 9. The method of any one of embodiments 1-5, wherein the CEC composition comprises about 2.5×10⁵ to about 1×10⁶ CECs.

Embodiment 10. The method of any one of embodiments 1-9, wherein the allogeneic human CECs are cultured prior to administration to the human subject.

Embodiment 11. The method of any one of embodiments 1-10, wherein the CEC composition is administered to an anterior chamber of an eye of the human subject.

Embodiment 12. The method of any one of embodiments 1-11, wherein the CEC composition is administered to the human subject as a one-time procedure.

Embodiment 13. The method of any one of embodiments 1-12, wherein the CEC composition and the Rho-associated, coiled-coil containing protein kinase inhibitor are administered as a combination composition.

Embodiment 14. The method of embodiment 13, wherein the combination composition is AURN001.

Embodiment 15. The method of any one of embodiments 1-12, wherein the CEC composition and the Rho-associated, coiled-coil containing protein kinase inhibitor are administered separately.

Embodiment 16. The method of any one of embodiments 1-15, wherein the CEC composition is neltependocel.

Embodiment 17. The method of any one of embodiments 1-16, wherein the Rho-associated, coiled-coil containing protein kinase inhibitor is Y-27632.

Embodiment 18. The method of embodiment 17, wherein about 100 microliters of Y-27632 is administered to the human subject.

Embodiment 19. A method for treating Fuchs' corneal dystrophy in human subjects in need thereof, comprising administering an effective amount of a corneal endothelial cell (CEC) composition and an effective amount of a Rho-associated protein kinase inhibitor to the human subject, wherein the composition comprises allogeneic human CECs, and wherein at least 50% of subjects treated achieve >15-letter improvement (>3-line gain using the Early Treatment Diabetic Retinopathy Standard (ETDRS) visual acuity test).

Embodiment 20. The method of embodiment 19, wherein the CEC composition comprises about 2.5×10⁵ CECs.

Embodiment 21. The method of embodiment 19, wherein the CEC composition comprises about 5×10⁵ CECs.

Embodiment 22. The method of embodiment 19, wherein the CEC composition comprises about 1×10⁶ CECs.

Embodiment 23. The method of embodiment 19, wherein the CEC composition comprises about 2.5×10⁵ to about 1×10⁶ CECs.

Embodiment 24. The method of any one of embodiments 19-23, wherein the allogeneic human CECs are cultured prior to administration to the human subjects.

Embodiment 25. The method of any one of embodiments 19-25, wherein the CEC composition is administered to an anterior chamber of an eye of each of the human subjects.

Embodiment 26. The method of any one of embodiments 19-25, wherein the CEC composition is administered to the human subjects as a one-time procedure for each.

Embodiment 27. The method of any one of embodiments 19-26, wherein the CEC composition and the Rho-associated, coiled-coil containing protein kinase inhibitor are administered as a combination composition.

Embodiment 28. The method of embodiment 27, wherein the combination composition is AURN001.

Embodiment 29. The method of any one of embodiments 19-28, wherein the CEC composition and the Rho-associated, coiled-coil containing protein kinase inhibitor are administered separately.

Embodiment 30. The method of any one of embodiments 19-29, wherein the CEC composition is neltependocel.

Embodiment 31. The method of any one of embodiments 19-30, wherein the Rho-associated, coiled-coil containing protein kinase inhibitor is Y-27632.

Embodiment 32. The method of embodiment 31, wherein about 100 microliters of Y-27632 is administered to the human subjects.

Embodiment 33. A method for treating a corneal endothelial disease in a human subject in need thereof, comprising administering an effective amount of a corneal endothelial cell (CEC) composition and an effective amount of a Rho-associated, coiled-coil containing protein kinase inhibitor to the human subject, wherein the composition comprises allogeneic human CECs, and wherein the corneal endothelial disease is a bullous keratopathy, a corneal edema, a corneal leukoma, a corneal endothelial inflammation, or a corneal dystrophy (optionally, Fuchs' dystrophy).

Embodiment 34. A method for treating or preventing a corneal endothelial disease (optionally, Fuchs' dystrophy) in a human subject in need thereof, comprising administering an effective amount of a corneal endothelial cell (CEC) composition and an effective amount of a Rho-associated, coiled-coil containing protein kinase inhibitor to the human subject, wherein the CEC composition comprises allogeneic human CECs, and wherein the human subject has corneal edema secondary to corneal endothelial dysfunction.

Embodiment 35. The method of embodiment 34, wherein the human subject achieves >15-letter improvement (>3-line gain using the Early Treatment Diabetic Retinopathy Standard (ETDRS) visual acuity test) from baseline in a best corrected visual acuity (BCVA) at six months.

Embodiment 36. The method of embodiment 34 or 35, wherein the human subject has a change from baseline in BCVA and in central corneal thickness (CCT) at six months.

Embodiment 37. The method of any one of embodiments 34-36, wherein the human subject has no loss from baseline in BCVA of more than 15 letters.

Embodiment 38. The method of any one of embodiments 33-37, wherein the CEC composition comprises about 2.5×10⁵ CECs.

Embodiment 39. The method of any one of embodiments 33-38, wherein the CEC composition comprises about 5×10⁵ CECs.

Embodiment 40. The method of any one of embodiments 33-38, wherein the CEC composition comprises about 1×10⁶ CECs.

Embodiment 41. The method of any one of embodiments 33-38, wherein the CEC composition comprises about 2.5×10⁵ to about 1×10⁶ CECs.

Embodiment 42. The method of any one of embodiments 33-41, wherein the allogeneic human CECs are cultured prior to administration to the human subject.

Embodiment 43. The method of any one of embodiments 33-42, wherein the CEC composition is administered to an anterior chamber of an eye of the human subject.

Embodiment 44. The method of any one of embodiments 33-43, wherein the CEC composition is administered to the human subject as a one-time procedure.

Embodiment 45. The method of any one of embodiments 33-44, wherein the CEC composition and the Rho-associated, coiled-coil containing protein kinase inhibitor are administered as a combination composition.

Embodiment 46. The method of embodiment 45, wherein the combination composition is AURN001.

Embodiment 47. The method of any one of embodiments 33-46, wherein the CEC composition and the Rho-associated, coiled-coil containing protein kinase inhibitor are administered separately.

Embodiment 48. The method of any one of embodiments 33-47, wherein the CEC composition is neltependocel.

Embodiment 49. The method of any one of embodiments 33-48, wherein the Rho-associated, coiled-coil containing protein kinase inhibitor is Y-27632.

Embodiment 50. The method of embodiment 49, wherein about 100 μM of Y-27632 is administered to the human subject.

Embodiment 51. A method for treating Fuchs' corneal dystrophy in human subjects in need thereof, comprising administering an effective amount of a corneal endothelial cell (CEC) composition and an effective amount of a Rho-associated protein kinase inhibitor to the human subject, wherein the composition comprises allogeneic human CECs, and wherein at least 50% of subjects treated achieve >15-letter improvement (>3-line gain using the Early Treatment Diabetic Retinopathy Standard (ETDRS) visual acuity test).

Embodiment 52. The method of embodiment 51, wherein the CEC composition comprises about 2.5×10⁵ CECs.

Embodiment 53. The method of embodiment 51, wherein the CEC composition comprises about 5×10⁵ CECs.

Embodiment 54. The method of embodiment 51, wherein the CEC composition comprises about 1×10⁶ CECs.

Embodiment 55. The method of embodiment 51, wherein the CEC composition comprises about 2.5×10⁵ to about 1×10⁶ CECs.

Embodiment 56. The method of any one of embodiments 51-55, wherein the allogeneic human CECs are cultured prior to administration to the human subjects.

Embodiment 57. The method of any one of embodiments 51-56, wherein the CEC composition is administered to an anterior chamber of an eye of each of the human subjects.

Embodiment 58. The method of any one of embodiments 51-57, wherein the CEC composition is administered to the human subjects as a one-time procedure for each.

Embodiment 59. The method of any one of embodiments 51-58, wherein the CEC composition and the Rho-associated, coiled-coil containing protein kinase inhibitor are administered as a combination composition.

Embodiment 60. The method of embodiment 59, wherein the combination composition is AURN001.

Embodiment 61. The method of any one of embodiments 51-60, wherein the CEC composition and the Rho-associated, coiled-coil containing protein kinase inhibitor are administered separately.

Embodiment 62. The method of any one of embodiments 51-61, wherein the CEC composition is neltependocel.

Embodiment 63. The method of any one of embodiments 51-62, wherein the Rho-associated, coiled-coil containing protein kinase inhibitor is Y-27632.

Embodiment 64. The method of embodiment 63, wherein about 100 μM of Y-27632 is administered to the human subjects.

Embodiment 65. A method for achieving an improvement from baseline in Best Corrected Visual Acuity (BCVA) at a timepoint post-treatment in a human subject with a corneal endothelial disease in need thereof, the method comprising administering to the human subject a therapeutically effective amount of human corneal endothelial cells (CECs) (e.g., CECs of a CEC composition disclosed herein) and a Rho-associated, coiled-coil containing protein kinase inhibitor, wherein the improvement is a ≥10-letter improvement in a Logarithm of the Minimum Angle of Resolution (LogMAR) chart or an equivalent improvement, and wherein the timepoint is at least 6 months post-treatment.

Embodiment 66. The method of embodiment 65, wherein the improvement is a ≥10-letter improvement in the Early Treatment Diabetic Retinopathy Standard (ETDRS) visual acuity test or an equivalent improvement.

Embodiment 67. The method of embodiment 65 or 66, wherein the improvement is a ≥15-letter improvement in the Early Treatment Diabetic Retinopathy Standard (ETDRS) visual acuity test or an equivalent improvement.

Embodiment 68. The method of any one of embodiments 65-67, wherein the equivalent improvement is an improvement in the Snellen chart.

Embodiment 69. A method for achieving a reduced central corneal thickness (CCT) from baseline at a timepoint post-treatment in a human subject with a corneal endothelial disease in need thereof, the method comprising administering to the human subject a therapeutically effective amount of human corneal endothelial cells (CECs) (e.g., CECs of a CEC composition disclosed herein) and a Rho-associated, coiled-coil containing protein kinase inhibitor, wherein the timepoint is at least 6 months post-treatment.

Embodiment 70. A method for achieving an improved subject-reported quality of life from baseline at a timepoint post-treatment in a human subject with a corneal endothelial disease in need thereof, the method comprising administering to the human subject a therapeutically effective amount of human corneal endothelial cells (CECs) (e.g., CECs of a CEC composition disclosed herein) and a Rho-associated, coiled-coil containing protein kinase inhibitor, wherein the subject-reported quality of life is measured with Visual Function Questionnaire-25 (VFQ-25) or an equivalent thereof, and wherein the timepoint is at least 6 months post-treatment.

Embodiment 71. The method of embodiment 70, wherein the subject-reported quality of life is measured with VFQ-25.

Embodiment 72. A method for achieving an increased endothelial cell density (ECD) from baseline at a timepoint post-treatment in a human subject with a corneal endothelial disease in need thereof, the method comprising administering to the human subject a therapeutically effective amount of human corneal endothelial cells (CECs) (e.g., CECs of a CEC composition disclosed herein) and a Rho-associated, coiled-coil containing protein kinase inhibitor, wherein the timepoint is at least 6 months post-treatment.

Embodiment 73. A method for achieving a reduced number of adverse effects of a corneal endothelial disease treatment in a human subject at a timepoint post-treatment, the method comprising administering to the human subject a therapeutically effective amount of human corneal endothelial cells (CECs) (e.g., CECs of a CEC composition disclosed herein) and a Rho-associated, coiled-coil containing protein kinase inhibitor, wherein the timepoint is at least 6 months post-treatment, and wherein the adverse effects comprise one or more of ocular hypertension, conjunctival hemorrhage, epithelial defect, ocular pain, cystoid macular edema, or corneal abrasion.

Embodiment 74. A method for achieving a reduced number of rescue surgeries in a corneal disease treatment in a human subject at a timepoint post-treatment, the method comprising administering to the human subject a therapeutically effective amount of human corneal endothelial cells (CECs) (e.g., CECs of a CEC composition disclosed herein) and a Rho-associated, coiled-coil containing protein kinase inhibitor, wherein the timepoint is at least 6 months post-treatment, and wherein the rescue surgeries are selected from Descemet Membrane Endothelial Keratoplasty (DMEK), Descemet Stripping Endothelial Keratoplasty (DSEK), and Penetrating Keratoplasty (PKP).

Embodiment 75. A method for achieving a >3-line gain using the Early Treatment Diabetic Retinopathy Standard (ETDRS) visual acuity test from baseline in a best corrected visual acuity (BCVA) at a timepoint post-treatment in a human subject with a corneal endothelial disease in need thereof, comprising administering to the human subject a therapeutically effective amount of human corneal endothelial cells (CECs) (e.g., CECs of a CEC composition disclosed herein) and a Rho-associated, coiled-coil containing protein kinase inhibitor, wherein the timepoint is at least 6 months post-treatment.

Embodiment 76. The method of any of embodiments 65-75, wherein the human CECs comprise allogeneic human CECs.

Embodiment 77. The method of any of embodiments 65-76, wherein the corneal endothelial disease is a bullous keratopathy, a corneal edema, a corneal leukoma, a corneal endothelial inflammation, or a corneal dystrophy.

Embodiment 78. The method of embodiment 77, wherein the corneal endothelial disease is Fuchs' corneal dystrophy or bullous keratopathy.

Embodiment 79. The method of embodiment 78, wherein the corneal endothelial disease is Fuchs' corneal dystrophy.

Embodiment 80. The method of any of embodiments 65-79, wherein the human subject has corneal edema secondary to corneal endothelial dysfunction.

Embodiment 81. The method of any of embodiments 65-80, wherein the human subject is within an age group (in years) of 2 and 99.

Embodiment 82. The method of embodiment 81, wherein the human subject is within an age group (in years) of 47 and 99.

Embodiment 83. The method of embodiment 81, wherein the human subject is within an age group (in years) of 52 and 94.

Embodiment 84. The method of embodiment 81, wherein the human subject is within an age group (in years) of 62 and 94.

Embodiment 85. The method of any of embodiments 65-84, wherein the therapeutically effective amount of human CECs comprise about 2.5×10⁵ CECs.

Embodiment 86. The method of any of embodiments 65-84, wherein the therapeutically effective amount of human CECs comprises about 5×10⁵ CECs.

Embodiment 87. The method of any of embodiments 65-84, wherein the therapeutically effective amount of human CECs comprises about 1×10⁶ CECs.

Embodiment 88. The method of any of embodiments 65-84, wherein the therapeutically effective amount of human CECs comprises about 2.5×10⁵ to about 1×10⁶ CECs.

Embodiment 89. The method of any of embodiments 65-84, wherein the therapeutically effective amount of human CECs comprise more than 2.5×10⁵ CECs.

Embodiment 90. The method of any of embodiments 65-84, wherein the therapeutically effective amount of human CECs comprises more than 5×10⁵ CECs.

Embodiment 91. The method of any of embodiments 65-84, wherein the therapeutically effective amount of human CECs comprises more than 1×10⁶ CECs.

Embodiment 92. The method of any of embodiments 65-91, wherein the human CECs are cultured prior to administration to the human subject.

Embodiment 93. The method of any of embodiments 65-92, wherein the human CECs and the Rho-associated, coiled-coil containing protein kinase inhibitor are administered as a single composition.

Embodiment 94. The method of any of embodiments 65-93, wherein the human CECs and the Rho-associated, coiled-coil containing protein kinase inhibitor are administered via separate compositions.

Embodiment 95. The method of embodiment 93 or 94, wherein the composition(s) are administered to an anterior chamber of an eye of the human subject.

Embodiment 96. The method of any of embodiments 93-95, wherein the composition(s) are administered to the human subject as a one-time procedure.

Embodiment 97. The method of any of embodiments 65-96, wherein the Rho-associated, coiled-coil containing protein kinase inhibitor is Y-27632.

Embodiment 98. The method of embodiment 97, wherein about 9.6 μg of Y-27632 is administered to the human subject.

Embodiment 99. The method of embodiment 97 or 98, wherein the Y-27632 is administered at a concentration of about 100 μM.

Embodiment 100. The method of any of embodiments 65-99, wherein the human subject has a change from baseline in BCVA and in CCT at six and/or twelve months post-treatment.

Embodiment 101. The method of any of embodiments 65-99, wherein the timepoint is at least 7 months post-treatment, at least 8 months post-treatment, at least 9 months post-treatment, at least 10 months post-treatment, at least 11 months post-treatment, or at least 12 months post-treatment.

Embodiment 102. A method for treating a corneal endothelial disease in a human subject in need thereof, comprising administering to the human subject about or more than 1×10⁶ corneal endothelial cells (CECs) (e.g., CECs of a CEC composition disclosed herein) and an effective amount of a Rho-associated, coiled-coil containing protein kinase inhibitor (optionally, Y-27632), wherein the CECs are allogeneic human CECs, wherein the administering is via a single dose or procedure.

Embodiment 103. The method of embodiment 102, wherein the corneal endothelial disease is Fuchs' dystrophy.

Embodiment 104. A method for treating corneal edema associated with a corneal endothelial dysfunction in a human subject in need thereof, comprising administering to the human subject about or more than 1×10⁶ corneal endothelial cells (CECs) (e.g., CECs of a CEC composition disclosed herein) and an effective amount of a Rho-associated, coiled-coil containing protein kinase inhibitor (optionally, Y-27632), wherein the CECs are allogeneic human CECs, wherein the administering is via a single dose or procedure.

Embodiment 105. The method of embodiment 104, wherein the corneal endothelial dysfunction is Fuchs' dystrophy.

Embodiment 106. The method of any one of embodiments 102-105, wherein the Rho-associated, coiled-coil containing protein kinase inhibitor is Y-27632, and the effective amount of Y-27632 is about 100 μM.

Embodiment 107. The method of any one of embodiments 102-106, wherein the method is effective to achieve and/or maintain a therapeutic improvement at least or more than 6 months or 12 months post-treatment.

Embodiment 108. A method for treating corneal edema associated with a corneal endothelial dysfunction (optionally, wherein the corneal endothelial dysfunction is Fuchs' dystrophy) in a human subject in need thereof, comprising administering to the human subject about or more than 1×10⁶ corneal endothelial cells (CECs) (e.g., CECs of a CEC composition disclosed herein) and 100 μM of Y-27632, wherein the CECs are allogeneic human CECs, wherein administering is via a single dose or procedure, and wherein the method is effective to achieve and/or maintain a therapeutic improvement at least or more than 6 months or 12 months post-treatment.

Embodiment 109. The method of embodiment 107 or 108, wherein the therapeutic improvement is an improvement in Best Corrected Visual Acuity (BCVA) which is a ≥15-letter improvement or >3-line gain in the Early Treatment Diabetic Retinopathy Standard (ETDRS) visual acuity test.

Embodiment 110. The method of embodiment 107 or 108, wherein the therapeutic improvement is an improvement in mean BCVA or a reduced central corneal thickness.

Embodiment 111. The method of embodiment 107 or 108, wherein the therapeutic improvement is an improvement in the subject-reported quality of life, optionally as measured using Visual Function Questionnaire-25 (VFQ-25).

Embodiment 112. The method of embodiment 107 or 108, wherein the therapeutic improvement is a reduced number of needed rescue surgeries for the disease or dysfunction.

Embodiment 113. A method of achieving an improvement in Best Corrected Visual Acuity (BCVA) in a human subject with corneal endothelial disease or dysfunction in need thereof (optionally, wherein the corneal endothelial dysfunction is Fuchs' dystrophy), comprising administering to the human subject about or more than 1×10⁶ corneal endothelial cells (CECs) (e.g., CECs of a CEC composition disclosed herein) and an effective amount of a Rho-associated, coiled-coil containing protein kinase inhibitor (optionally, 100 μM of Y-27632), wherein the CECs are allogeneic human CECs, wherein the human subject has corneal edema, wherein the improvement is a ≥15-letter improvement or >3-line gain in the Early Treatment Diabetic Retinopathy Standard (ETDRS) visual acuity test, and wherein the improvement is achieved and/or maintained at least or more than 6 months or 12 months post-treatment.

Embodiment 114. The method of embodiment 113, wherein the administering is via a single dose or procedure.

Embodiment 115. The method of any one of embodiments 102-114, wherein the administering is to an anterior chamber of an eye of the human subject.

Embodiment 116. The method of any one of embodiments 102-115, which does not result in a graft rejection.

Embodiment 117. The method of any one of embodiments 102-116, which causes no or substantially no ocular adverse effects (optionally, no or substantially no ocular hypertension, conjunctival hemorrhage, epithelial defect, ocular pain, cystoid macular edema, or corneal abrasion).

Embodiment 118. The method of any one of embodiments 102-117, wherein the CECs comprise at least 70%, at least 75%, at least 80%, at least 85%, at least 90%, at least 95%, at least 97%, at least 98%, or at least 99% (optionally, at least or more than 80%, 85% or 90%) of human corneal endothelial cells that are CD166 positive, CD105 negative to weakly positive, and CD44 negative to weakly positive; optionally at least or more than 80% or 85% of human corneal endothelial cells that are CD166 positive, CD105 negative, and CD44 negative.

Embodiment 119. The method of any one of embodiments 102-117, wherein the CECs comprise at least 70%, at least 75%, at least 80%, at least 85%, at least 90%, at least 95%, at least 97%, at least 98%, or at least 99% (optionally at least or more than 80%, 85% or 90%) of human corneal endothelial cells that are CD166 positive, CD105 negative to weakly positive, CD24 negative, and CD44 negative to weakly positive; optionally at least or more than 80% or 85% of human corneal endothelial cells that are CD166 positive, CD105 negative, CD24 negative, and CD44 negative.

Embodiment 120. The method of any one of embodiments 102-117, wherein the CECs comprise at least 70%, at least 75%, at least 80%, at least 85%, at least 90%, at least 95%, at least 97%, at least 98%, or at least 99% (optionally at least or more than 80%, 85% or 90%) of human corneal endothelial cells that are CD166 positive, CD105 negative to weakly positive, CD26 negative, and CD44 negative to weakly positive; optionally at least or more than 80% or 85% of human corneal endothelial cells that are CD166 positive, CD105 negative, CD26 negative, and CD44 negative.

Embodiment 121. The method of any one of embodiments 1 to 120, wherein the CECs are not in a freshly prepared composition.

Embodiment 122. The method of embodiment 121, wherein the non-fresh CEC composition is administered to the human subject at most about 1, about 2, or about 3 days since preparation.

Embodiment 123. The method of embodiment 121, wherein the non-fresh CEC composition is stored at 2-8 degrees Celsius.

Embodiment 124. The method of any one of embodiments 121-123, wherein the non-fresh CEC composition comprises a Rho-associated, coiled-coil containing protein kinase inhibitor (optionally, Y-27632 or 100 μM of Y-27632).

### Example

### Example 1: Phase I/II Clinical Trial for AURN001

### Overview

AURN001 is Aurion's allogeneic cell therapy candidate designed to treat corneal edema secondary to corneal endothelial disease. AURN001 is a combination product, comprising neltependocel and Y-27632. AURN-001 is formulated and supplied as a suspension with human serum albumin (HSA) and Dulbecco's Modified Eagle Medium (DMEM). AURN001 is currently being studied in a Phase 1/2 Study of AURN001 in Subjects with Corneal Edema Secondary to Corneal Endothelial Dysfunction (ABA-1) (CLARA) (ClinicalTrials.gov ID NCT06041256). The present example describes the design of the CLARA trial and presents results analysis at month 6 following treatment.

Corneal edema secondary to endothelial dysfunction poses a significant threat to vision, impacting millions globally. The condition arises from the loss or degradation of corneal endothelial cells, which do not regenerate naturally. Traditional treatments include corneal transplants like PKP, DMEK, or DSAEK, which face challenges such as limited donor supply and post-operative recovery requirements.

Despite the effectiveness of transplants, the scarcity of donor corneas remains a critical issue, with one donor cornea available for every 70 diseased eyes. Additionally, recovery from transplant surgery often necessitates extended periods of post-operative care. AURN001 offers a promising alternative, potentially transforming the landscape of corneal endothelial disease treatment by overcoming the scarcity of donor corneas.

The goal of the CLARA clinical trial is to compare different doses of AURN001 in patients with corneal edema secondary to corneal endothelial dysfunction. The main questions the clinical trial aims to answer are whether AURN001 is effective and safe. Participants received a single injection of AURN001. A comparison between low, medium, and high doses of AURN001 against the contribution of each element, cells alone and Y27632 alone, was conducted to determine the effects on corneal edema. The primary endpoint is the percentage of subjects achieving a three-line improvement in vision at six months.

The following inclusion and exclusion criteria were used when enrolling patients in the study.

### Inclusion Criteria Included:

Have a diagnosis of corneal edema secondary to corneal endothelial dysfunction, requiring surgery (full- or partial-thickness endothelial keratoplasty).

BCVA between 65 Early Treatment of Diabetic Retinopathy Study (ETDRS) letters (i.e., 0.4 LogMAR or approximate 20/50 Snellen equivalent) and 5 ETDRS letters (i.e., 1.6 LogMAR or approximate 20/800 Snellen equivalent).

### Exclusion Criteria Included:

Have pre-operative corneal epithelial, sub-epithelial or stromal scarring or other opacity that is paracentral/central and visually significant, but not suspected to be secondary to corneal endothelial disease with the potential to improve from treatment in the study eye.

Have history or presence of an ocular disease other than corneal endothelial dysfunction that could affect vision or safety assessments.

The study design was a treatment study with randomized allocation into groups. Quadruple masking was used (Participant, Care Provider, Investigator, Outcomes Assessor). The treatment groups were:

| Participant Group/Arm | Intervention/Treatment |
|---|---|
| Experimental: AURN001 High | Combination Product: AURN001 |
| Neltependocel High and Rho- | 1.0 × 10⁶ Corneal Endothelial |
| associated protein kinase | Cells and 100 μM Y27632 |
| Experimental: AURN001 Medium | Combination Product: AURN001 |
| Neltependocel High and Rho- | 5 × 10⁵ Corneal Endothelial |
| associated protein kinase | Cells and 100 μM Y27632 |
| Experimental: AURN001 Low | Combination Product: AURN001 |
| Neltependocel High and Rho- | 2.5 × 10⁵ Corneal Endothelial |
| associated protein kinase | Cells and 100 μM Y27632 |
| Experimental: Neltependocel - High | 1.0 × 10⁶ Corneal Endothelial |
| Neltependocel- High | Cells |
| Experimental: ROCK | 100 μM Y27632 |
| Rho-associated protein kinase | |
| (ROCK) | |

Primary outcome measure was:

| Outcome | | Time |
|---|---|---|
| Measure | Measure Description | Frame |
| BCVA - 15-letter | Response, defined as a ≥ 15-letter | Month |
| improvement (3- | improvement (3-line gain) from baseline in | 6 |
| line gain) | best-corrected visual acuity (BVCA) | |

The study and outcome are summarized with further details provided below.

Background: 97 subjects with corneal edema secondary to corneal endothelial dysfunction were enrolled across 20 sites (US & Canada). Overall, baseline best corrected visual acuity (BCVA) and central corneal thickness (CCT) were better relative to those reported in previous OUS studies. Baseline BCVA and CCT varied across groups, with the high-dose group having best baseline BCVA and CCT.

Efficacy: A dose-dependent response was evident for the primary endpoint (percentage of subjects with 3 lines BCVA gained), with the high-dose group achieving the best outcomes at Month 6 with a 50% response rate (FAS) (56%-PP). A dose-dependent response was evident for the secondary endpoints (change from baseline in BCVA and CCT). Demonstrated quality of life benefit as measured by improvements in visual function questionnaire (VFQ-25) (composite and construct scores). A dose-dependency was observed in the percentage of subjects requiring rescue surgery with the high-dose group achieving the lowest rescue rate at Month 6 with a 0% rescue rate.

Efficacy outcomes for various groups were as follows:
High-dose group (1.0×10\hat{ }6 Cells+100 μM Y-27632) achieved statistical significance (p=0.020) in the primary endpoint (proportion of subjects with 3-line improvement) and secondary endpoints (change from baseline in BCVA and CCT) relative to 100 μM Y-27632.
1.0×10\hat{ }6 Cells+100 μM Y-27632:59 letters (20/60) and 629 microns vs. overall population: 53.5 letters (20/90) and 677 microns.
Dose response seen for 3-Line gainers: 14.3% (100 μM Y-27632), 44.4% (1.0×10\hat{ }6 Cells), 10.5% (2.5×10\hat{ }5 Cells+100 μM Y-27632), 36.8% (5.0×10\hat{ }5 Cells+100 μM Y-27632) and 50% (1.0×10\hat{ }6 Cells+100 μM Y-27632).
Dose response seen, with 1.0×10\hat{ }6 Cells+100 μM Y-27632 and 1.0×10\hat{ }6 Cells having greater overall efficacy (mean BCVA & CCT) than the 5.0×10\hat{ }5 Cells+100 μM Y-27632, 2.5×10\hat{ }5 Cells+100 μM Y-27632 and 100 μM Y-27632 groups.
Mean change from baseline in BCVA at 6 months was statistically significant (p=<0.05) in 1.0×10\hat{ }6 Cells+100 μM Y-27632 and 1.0×10\hat{ }6 Cell groups relative to the 100 UM Y-27632 group.
ECD data was not consistently measurable.
Safety: The drug product was generally well tolerated with a favorable safety profile across all study groups.
Safety outcomes for various groups were as follows:
   All doses were generally safe and well tolerated with no ocular serious adverse events (SAEs).
   Dose response seen in the rescue population (n=25) with the majority of subjects being in the 100 μM Y-27632 group (n=13) and zero in 1.0×10\hat{ }6 Cells+100 μM Y-27632 group.
   No iris abnormalities were observed.
   Intra ocular pressure (IOP) was the most common adverse event (AE)—none observed with high dose group.

### Study Design and Demographics

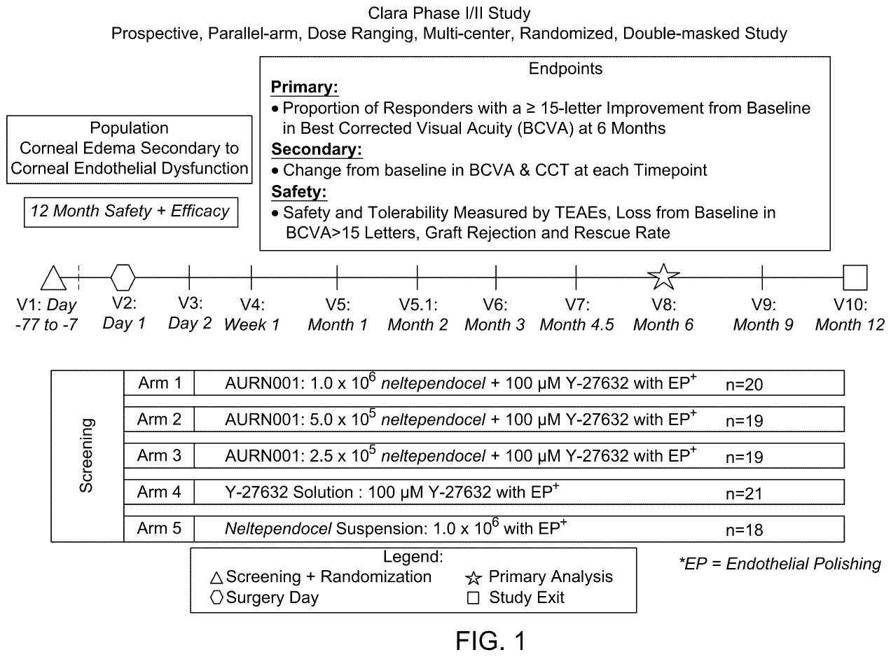

The design of the study can be readily understood by reference to FIG. 1 which provides the treatment groups and timelines of the study. Treatments were prepared before surgery on D1 (e.g., AURN001 formulated 12-24 hours or more before surgery and stored at 2-8° C. before use). As indicated in FIG. 1, subjects in each treatment group received endothelial polishing (EP) for the administration of each treatment. Briefly, the injection of the respective treatment into the eye was performed under local anesthesia. After creating a small incision at the corneal limbus, the corneal endothelium was polished to a diameter of approximately 8 mm. The respective treatment was injected into the anterior chamber. Immediately after completion of surgery, subjects lay face down for approximately 3 hours.

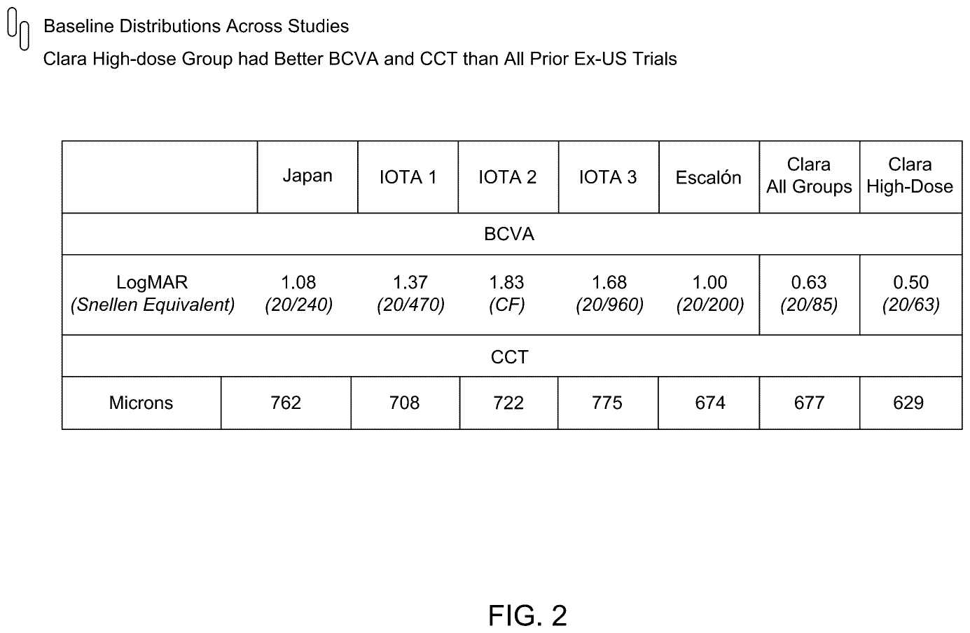

To better appreciate the results from the CLARA study, baseline BCVA and CCT were compared to baseline values in other studies to treat ocular disease. An overview is provided in FIG. 2. Subjects in the high-dose group of the CLARA trail (receiving 1.0×10⁶ Corneal Endothelial Cells and 100 μM Y27632) had better baseline BCVA (0.50 LogMAR) and lower CCT (629 microns).

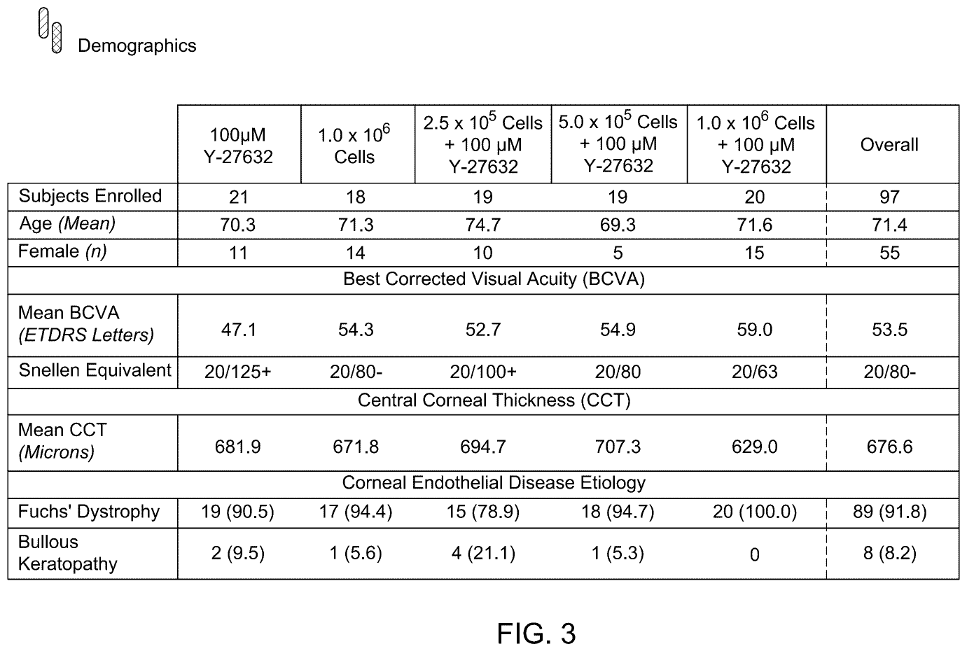

In the CLARA study, subjects were randomized into five groups. The demographics for each group and the population overall are provided in FIG. 3. A majority of subjects in each treatment group had Fuch's dystrophy and a minority had bullous keratopathy.

### Efficacy Outcomes

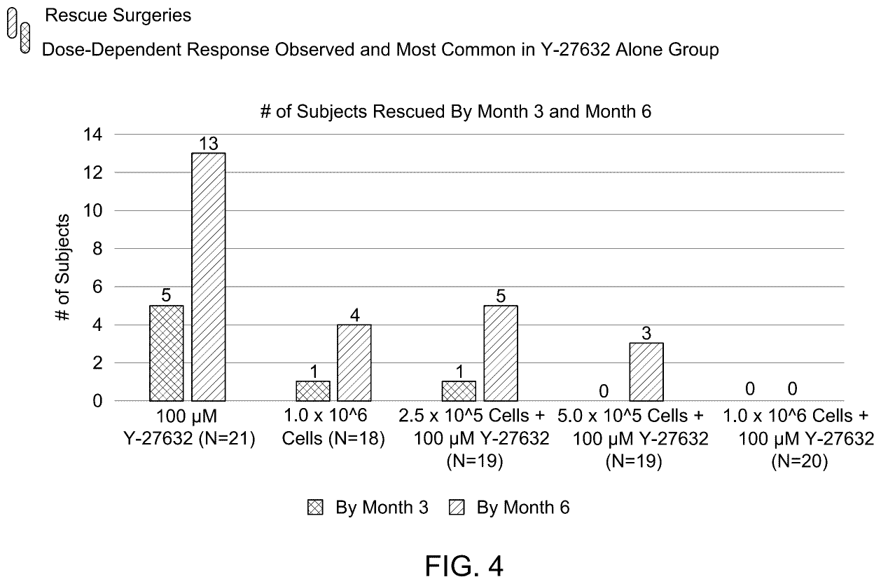

The efficacy of the various treatment regimens can be assessed, for example, by the number of rescue surgeries required after treatment with the various regimens. As shown in FIG. 4, subjects treated with cell therapy, either alone or in combination with 100 μM Y-27632 required fewer rescue surgeries at both the 3 month and 6 month time points as compared to subjects treated with 100 μM Y-27632 alone. None of the subjects treated with a combination of 1.0×10\hat{ }6 cells and 100 μM Y-27632 required rescue surgery during six months of the study. Fewer subjects in the medium-dose group required rescue surgery than in the low-dose group.

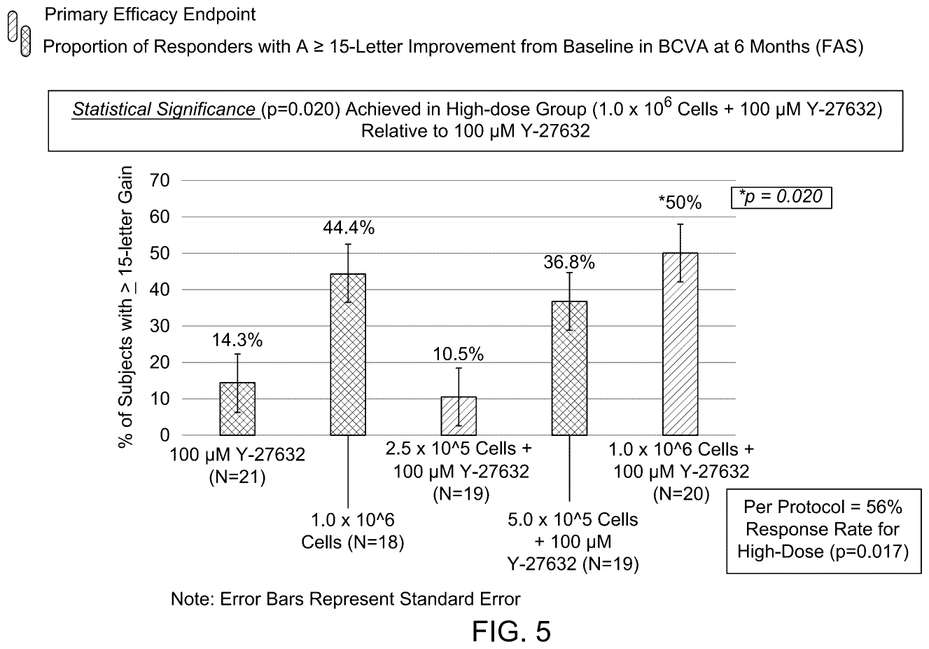

The efficacy of the various treatment regimens can be assessed, for example, by the primary efficacy endpoint of the study, i.e., the proportion of responders with a ≥15-letter improvement from baseline in BCVA at 6 months (FAS). The results are shown in FIG. 5. A statistically significant (p=0.020) outcome was achieved in High-dose group (1.0×10\hat{ }6 Cells+100 μM Y-27632) relative to 100 μM Y-27632. Overall, 50% of subjects achieved ≥15-letter improvement from baseline in BCVA at 6 months. In Y-27632 group, 14.3% of subjects showed ≥15-letter improvement. In Neltependocel-only (cell only) group, 44.4% subjects showed ≥15-letter improvement at month 6. Per Protocol Set (PPS) analysis, a subset of Full Analysis Set, excluding subjects that had protocol deviations, yielded a response rate of Per Protocol=56% Response Rate for High-Dose (p=0.017).

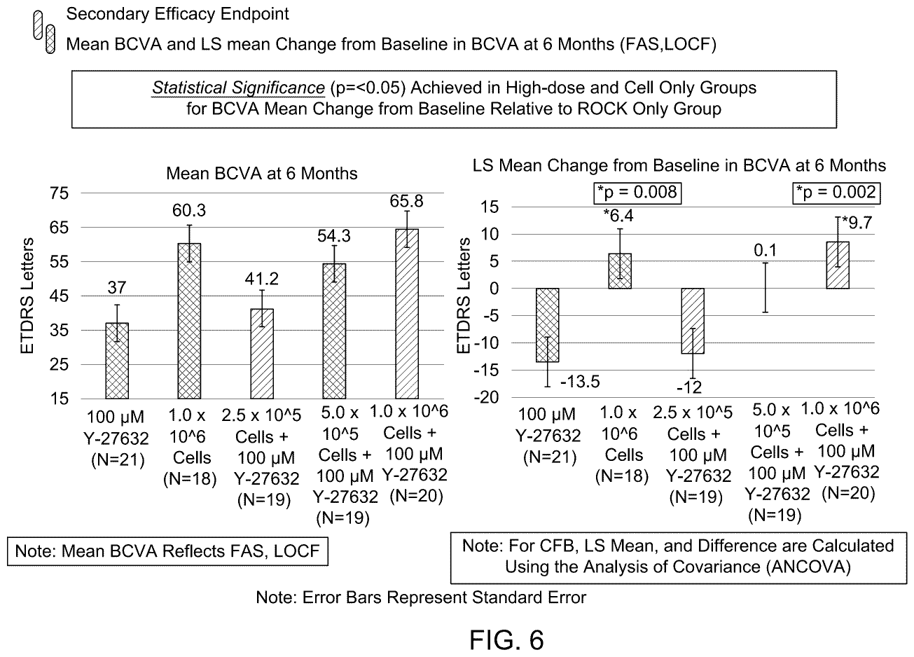

The efficacy of the various treatment regimens can be assessed, for example, by the secondary efficacy endpoint of the study, i.e., the mean BCVA and LS mean change from baseline BCVA at 6 months (FAS, LOCF). The results are shown in FIG. 6. A statistically significant (p=<0.05) outcome was achieved in High-Dose and Cell Only Groups for BCVA Mean Change From Baseline Relative to ROCK Only Group. On average, the High-Dose group receiving 1.0×10⁶ Corneal Endothelial Cells and 100 M Y-27632 showed the highest mean BCVA (65.8 letters) at month 6 and the highest mean change in BCVA (9.7 letters) at month 6 compared to baseline. The Neltependocel-only (1.0×10⁶ cells only) group showed a mean BCVA of 60.3 letters at month 6 and a mean change in BCVA of 6.4 letters. The Y-27632 only group, the low-dose group, and the medium-dose group experienced negative mean BCVA changes or no changes.

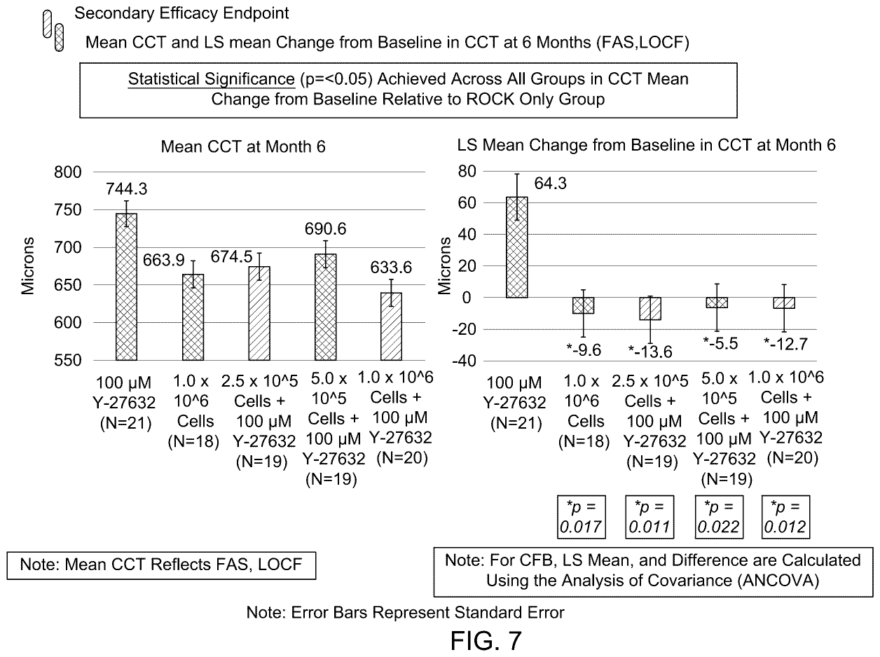

The efficacy of the various treatment regimens can be assessed, for example, by an alternate secondary efficacy endpoint of the study, i.e., the mean CCT and LS mean change from baseline CCT at 6 months (FAS, LOCF). The results are shown in FIG. 7. A statistically significant (p=<0.05) outcome was achieved in all groups for CCT Mean Change from Baseline relative to ROCK Only Group. The high-dose group had the lowest mean CCT at month 6, and a second largest decrease in mean CCT.

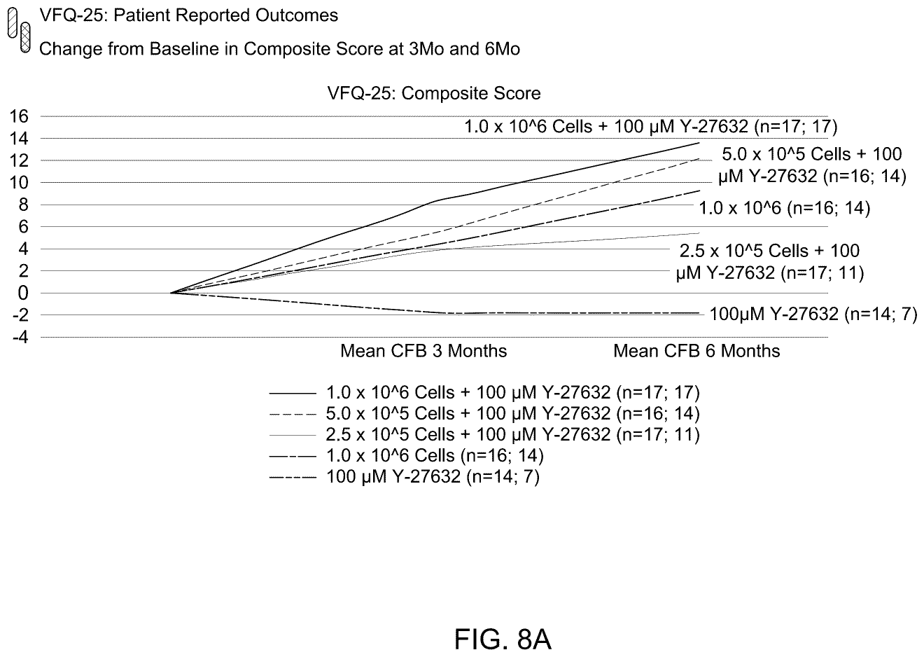

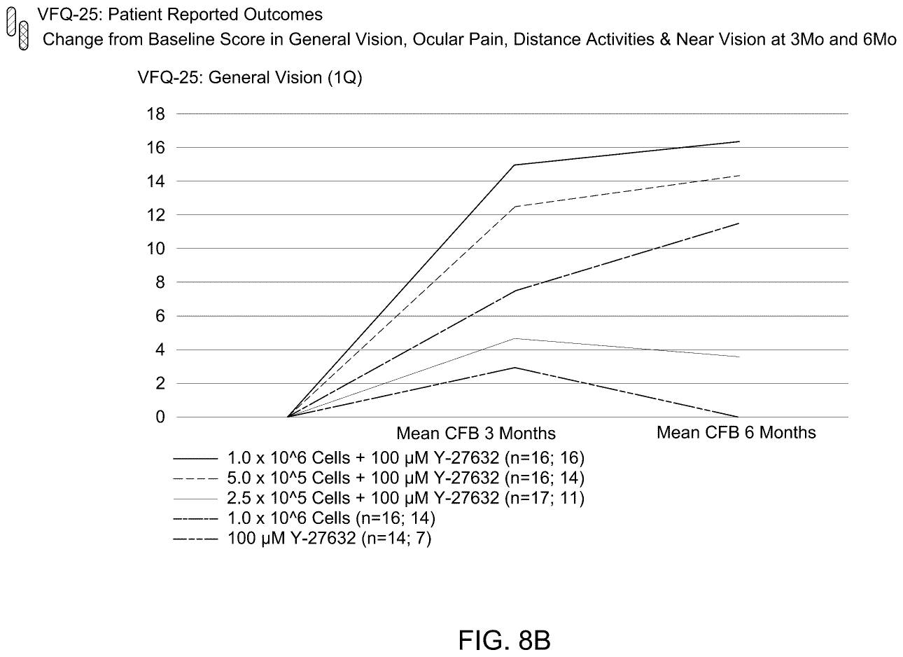

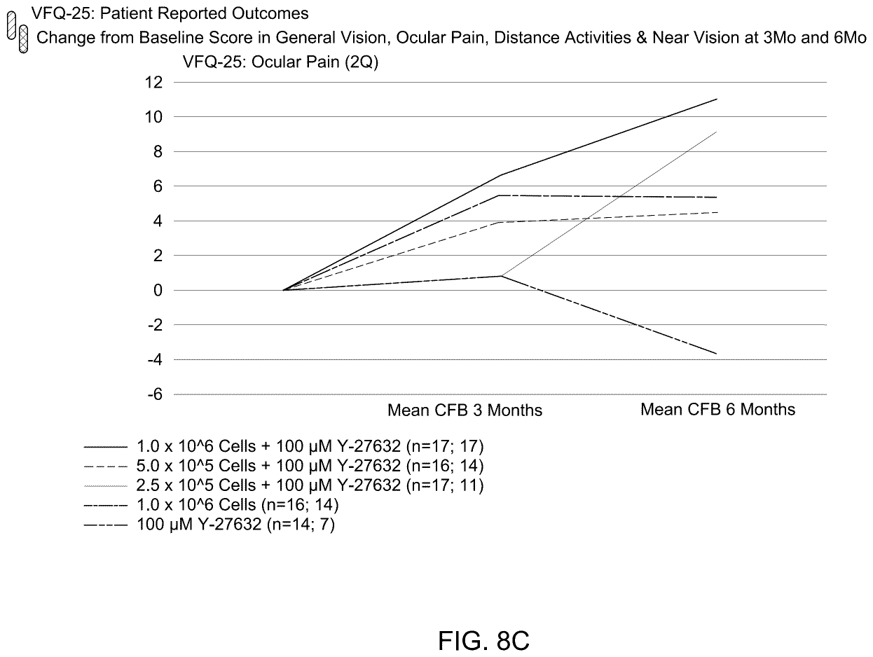

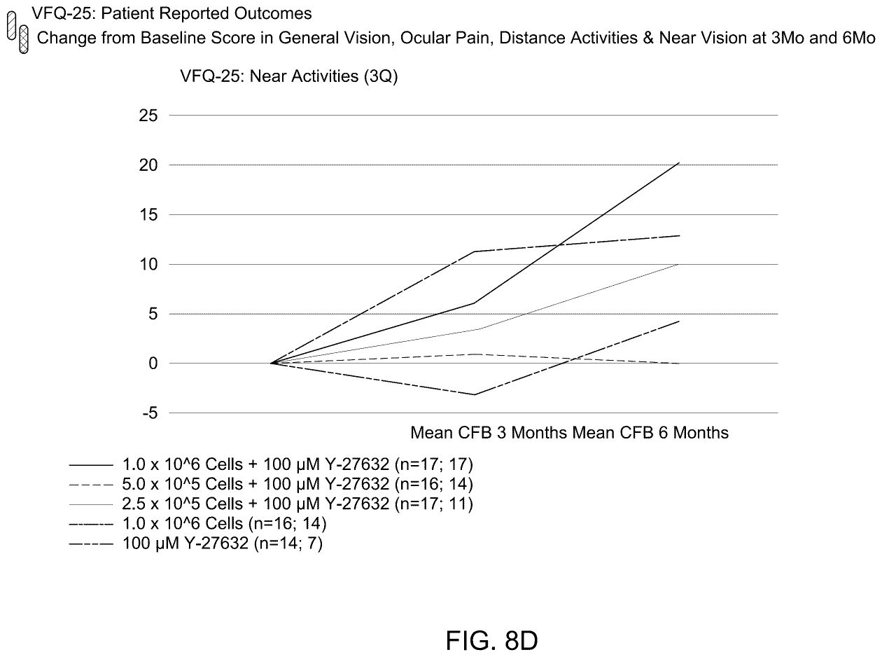

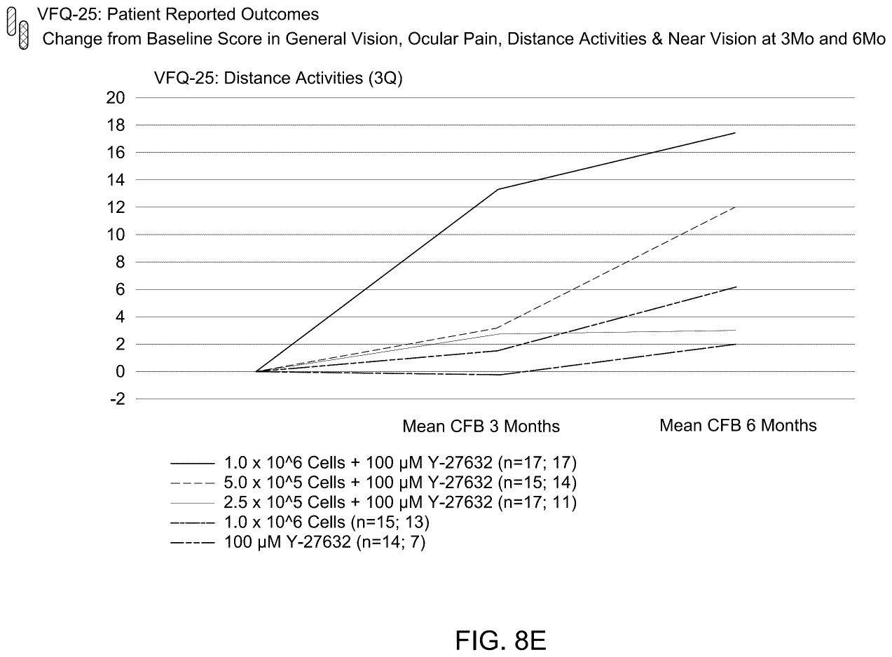

The efficacy of the various treatment regimens can be assessed, for example, by patient reported outcomes, as assessed using Visual Function Questionnaire 25 (VFQ-25), i.e., in the change in baseline composite score at 3 and 6 months. The assessment tool assigns 100 to the best possible score and 0 to the worst possible score. Therefore, an increase in the score is indicative of an increase in quality of life. The results for the composite score are shown in FIG. 8A. All treatment groups except for the Y-27632 only group experienced positive improvements in the VFQ-25 composite score while the high-dose group experienced the most improvement at month 3 and month 6. The results for various components of the score are shown in FIGS. 8B-8E. The high-dose group experienced the most improvement at month 6 relative to baseline for each component of the composite score.

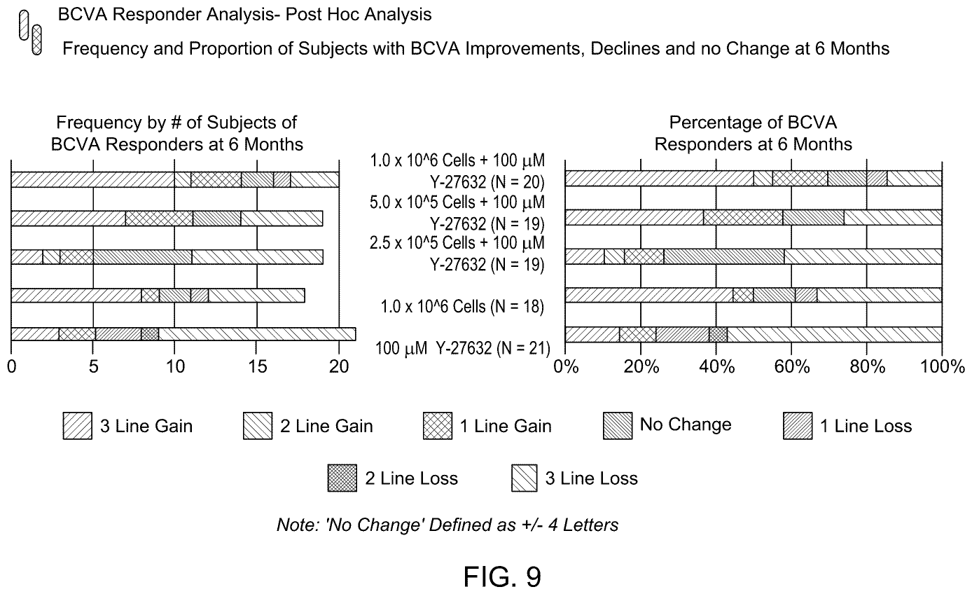

A post-hoc BCVA responder analysis was performed to visualize the study data in a different manner. The analysis was performed to determine the frequency and proportion of subjects with BCVA improvements, declines, or no change at 6 months. The results are shown in FIG. 9. As shown in FIG. 9, more subjects in the high-dose group experienced either an overall BCVA improvement or an 3-line gain (15-letter improvement) than the other treatment groups. A majority of subjects in the Y-27632 only group experienced negative BCVA changes at month 6.

### Safety Outcomes

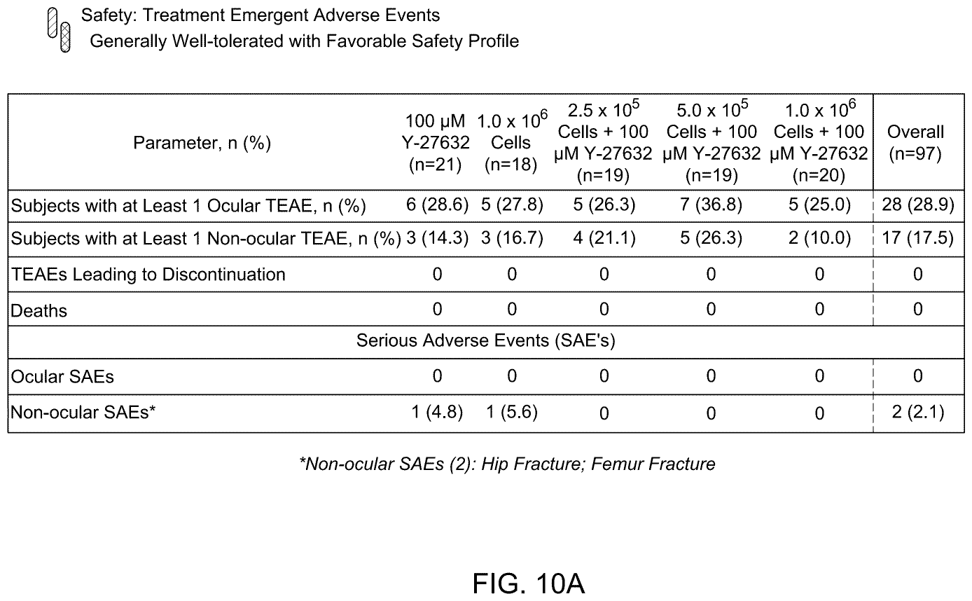

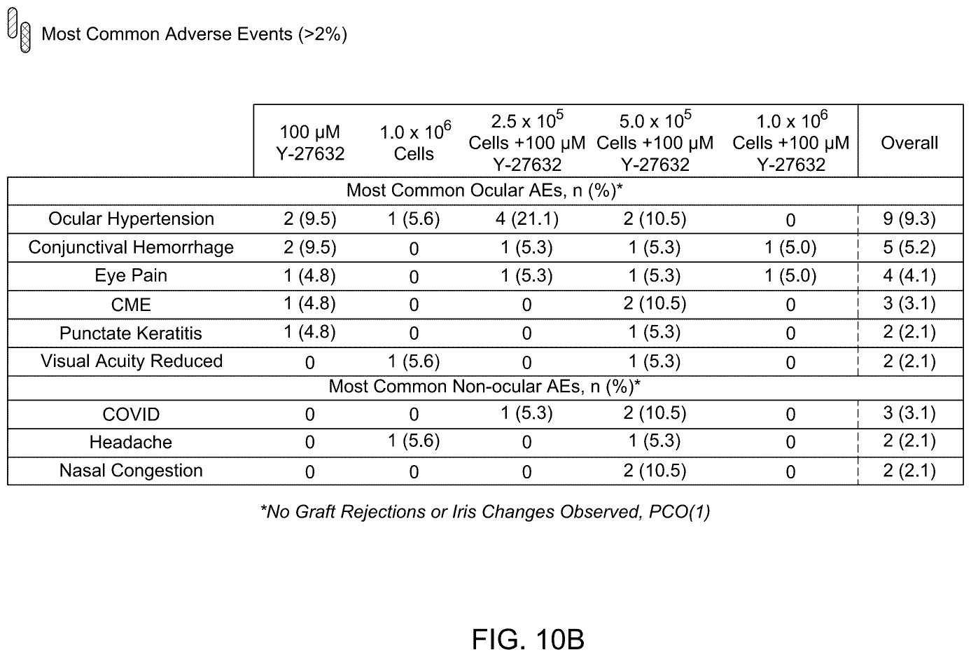

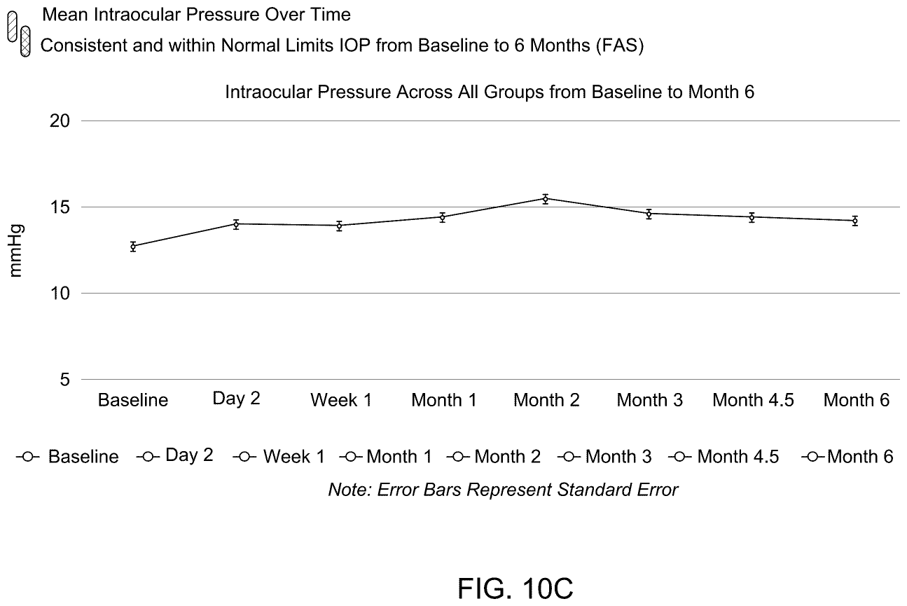

Clinical trials are performed to determine the safety of drug products and treatment regimens. Throughout the CLARA clinical trial, subjects were monitored for adverse events. The assessment of adverse events demonstrated that the drug product is generally well-tolerated with a favorable safety profile. Safety treatment emergent events are shown in FIG. 10A. The most common adverse events are shown in FIG. 10B. Mean intraocular pressure over time, monitored as a safety indicator, is shown in FIG. 10C.

The results provided herein demonstrate the safety and efficacy of all of the cell-based treatment regimens, particularly the safety and efficacy of the 1.0×10\hat{ }6 Cells+100 μM Y-27632 treatment regimen. The high-dose regimen resulted in the least treatment emergent adverse events and no serious adverse events.

### Additional Objectives and Outcomes

The CLARA clinical trial provided additional information to inform and facilitate further clinical studies.

The outcomes of CLARA inform future clinical development pathway including, selection of concentration of active arm 1.0×10\hat{ }6 Cells+100 μM Y-27632 (AURN001) to carry forward, establishment of control for pivotal studies; and determining statistical powering and study size for pivotal studies based on response rates seen in Phase I/II, 90%, with a plan for 3:1 randomization in Phase III study.

The study demonstrated successfully manufacture and distribution of the drug product from central CDMO to multiple sites in US and Canada (20 Sites).

The study validated clinical performance of AURN001 with an extended 48-hour shelf life (previously 6 hours). The extended shelf life could allow for inclusion of additional sites having additional time for transport of AURN001 to trial sites.

Also important to the inclusion of additional sites in future studies was the development and implementation surgical training program to ensure procedural consistency and further optimize surgical procedure (i.e., intraoperative AC maintenance). Enhance surgical efficiency: upgraded endothelial polisher and reduced procedure duration.

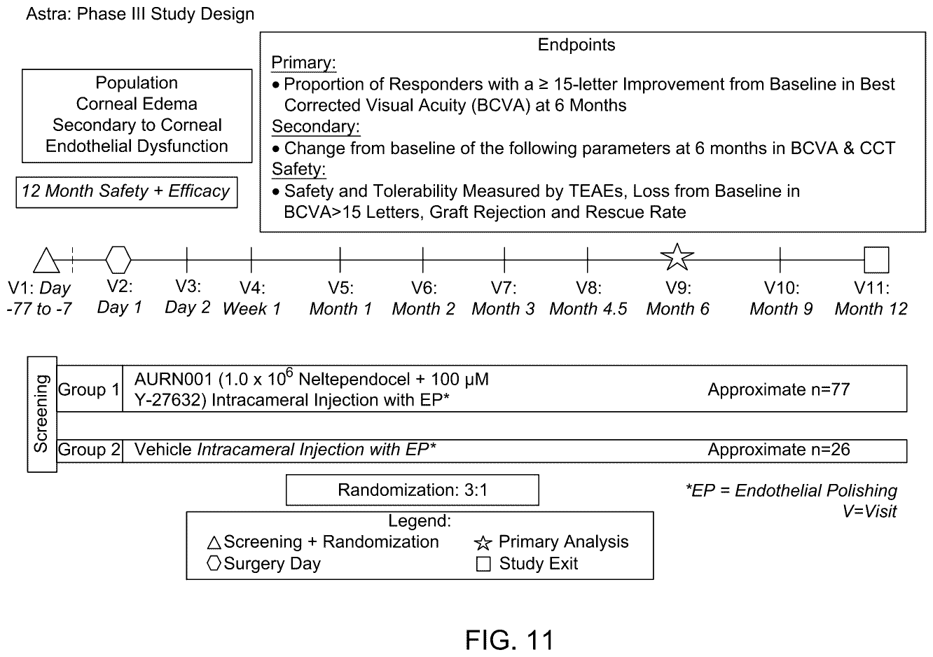

A proposed Phase III clinical trial is provided in FIG. 11.

### SUMMARY

AURN001 is a combination cell therapy product candidate comprised of neltependocel, allogeneic human corneal endothelial cells, and Y-27632, a Rho-associated, coiled-coil containing protein kinase inhibitor. AURN001 is intended to be administered to the anterior chamber of the eye as a one-time procedure.

The CLARA Phase 1/2 clinical trial (NCT06041256) is a prospective, multi-center, randomized, double-masked, parallel-arm dose-ranging clinical trial in subjects with corneal edema secondary to corneal endothelial dysfunction. The Phase 1/2 CLARA trial was designed to assess the safety, tolerability and efficacy of AURN001 for the treatment of corneal edema secondary to corneal endothelial dysfunction. Ninety-seven subjects were randomized at US and Canadian sites. The trial consists of five arms: three arms evaluating AURN001 (different doses of neltependocel in combination with Y-27632), one arm evaluating neltependocel monotherapy and one arm evaluating Y-27632 monotherapy. The primary endpoint is the proportion of responders with a >15-letter improvement (>3-line gain using the Early Treatment Diabetic Retinopathy Standard (ETDRS) visual acuity test) from baseline in best corrected visual acuity (BCVA) at six months. Key secondary endpoints include (i) change from baseline in BCVA and in central corneal thickness (CCT) at six months and (ii) safety and tolerability as measured by treatment-emergent adverse events (TEAEs), loss from baseline in BCVA >15 letters, graft rejection and rescue rate.

The primary endpoint, the percentage of subjects with three lines of BCVA gained at six months, showed statistically significant improvement in the high-dose AURN001 arm (50%, p=0.020), as compared to the Y-27632 only arm (14%). Overall, 70% of subjects in the high-dose AURN001 arm achieved one or more lines of improvement in BCVA.

A secondary endpoint, change in BCVA at six months, using the full analysis set (FAS) population (LOCF, LS Mean¹) showed a statistically significant improvement for the high-dose AURN001 arm (p=0.002) and for the neltependocel only arm (p=0.008), as compared to the Y-27632 only arm. ¹ LOCF: Last Observed Carry Forward statistical analysis, Ls Mean: Least Squares Mean statistical analysis

Separately, an improvement in quality of life using the Visual Function Questionnaire (VFQ-25) was noted with the greatest benefit being seen in the high-dose AURN001 arm. Similar trends were seen in most of the other VFQ-25 constructs.

Doses from all five treatment arms were generally well tolerated with favorable safety profiles. In the Y-27632 only arm, there were 13 rescues (N=21 subjects). Overall, there were 28 subjects with ocular TEAEs and 17 subjects with non-ocular TEAEs. There was no dose response in frequency of adverse events (AEs). There were no ocular serious adverse events (SAEs) reported and two non-ocular SAEs (hip fracture and femur fracture). The most frequently reported (>3%) ocular AEs were ocular hypertension (9.3%), conjunctival hemorrhage (5.2%) and eye pain (3.1%), and the most frequently reported non-ocular AE was COVID-19 (3.1%).

In the CLARA trial, 97 subjects were enrolled across 20 sites in the United States and Canada. At baseline across all arms, the mean age was 71.4 years with 55% being female subjects. Baseline mean BCVA was 53.5 letters ETDRS (20/80-Snellen) and CCT was 676.6 microns.

| | | | “Low” | “Medium” | “High” |
|---|---|---|---|---|---|
| | | | AURN001: | AURN001: | AURN001: |
| | | | 2.5 × 10⁵ | 5 × 10⁵ | 1 × 10⁶ |
| | | 1.0 × 10⁶ | Neltepenmdocel + | Neltepenmdocel + | Neltepenmdocel + |
| | Y-27632 | Neltepenmdocel | 100μ | 100μ | 100μ |
| | Only | Only | Y-27632 | Y-27632 | Y-27632 |
| Number of | 21 | 18 | 19 | 19 | 20 |
| subjects | | | | | |
| Baseline | 47.1 | 54.3 | 52.7 | 54.9 | 59.0 |
| BCVA | | | | | |
| (ETDRS) | | | | | |
| Baseline | 0.76 | 0.61 | 0.65 | 0.60 | 0.52 |
| BCVA | | | | | |
| (LogMAR) | | | | | |
| Baseline | 20/125+ | 20/80− | 20/100+ | 20/80 | 20/63 |
| BCVA | | | | | |
| (Snellen) | | | | | |
| Baseline | 681.9 | 671.8 | 694.7 | 707.3 | 629 |
| CCT | | | | | |
| (microns) | | | | | |

### Example 2: Phase I/II Clinical Trial for AURN001-12 Month Analysis

As described in FIG. 1, a Study Exit analysis was performed at month 12 in the CLARA trial of the subjects described in Example 1, using the described parameters.

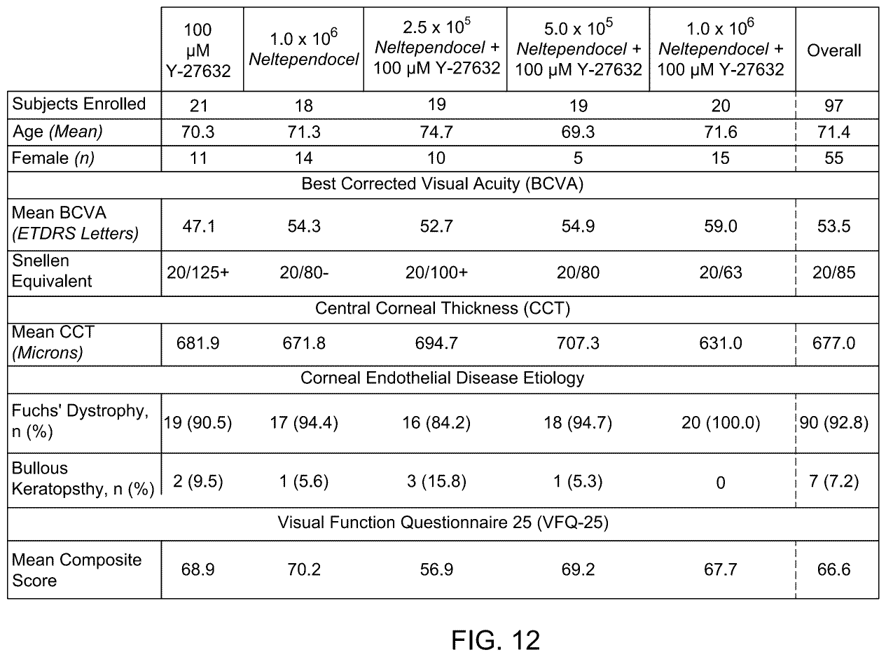

The demographics for each group and the population overall were re-analyzed and provided in FIG. 12. Specifically, the baseline mean CCT in the high-dose group receiving 1.0×10⁶ Cells and 100 μM Y-27632 was adjusted to 631 microns from 629 microns. Of the subjects in the low-dose group, 16 subjects were classified to have Fuch's dystrophy and 3 to have bullous keratopahy. Baseline subject reported VFQ-25 was 66.6 points across all treatment groups.

### Efficacy Outcomes

The efficacy of the various treatment groups at month 12 was assessed.

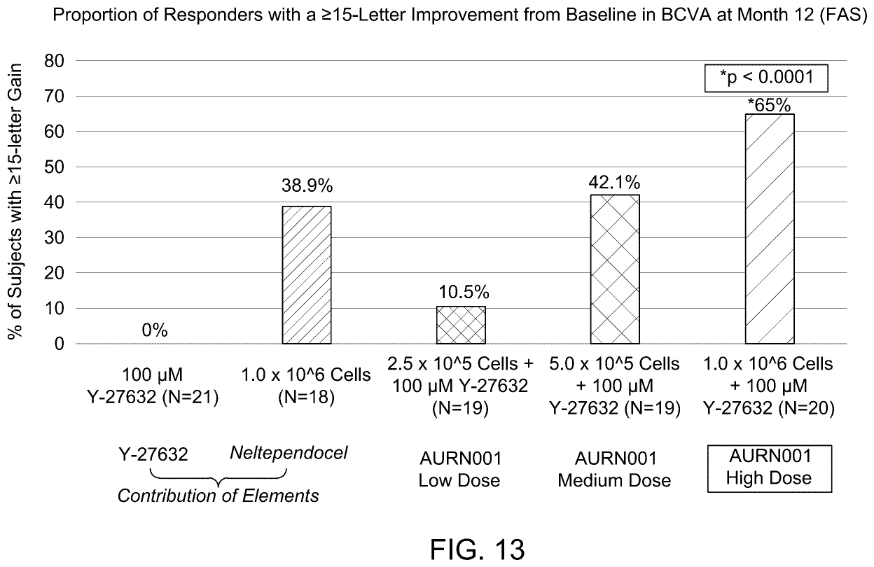

Similar to the month 6 analysis, efficacy of the various treatment regimens at month 12 was assessed, for example, by the primary efficacy endpoint, i.e., the proportion of responders with a ≥15-letter improvement from baseline in BCVA at 12 months (FAS). As shown in FIG. 13, 65% of subjects in the high-dose group achieved ≥15-letter improvement from baseline in BCVA at 12 months and demonstrated a significant difference from the Y-27632 only group, improving from the 50% at month 6. In contrast, no subjects maintained ≥15-letter BCVA improvement at month 12 in the Y-27632 only group, whereas 14.3% demonstrated improvement at prior month 6 time point. Subjects in Neltependocel only (cell only) group, the low-dose group, and the medium-dose group experienced smaller changes in the proportion of subjects demonstrating ≥15-letter BCVA improvement, with medium-dose subjects showing a small increase in the proportion and Neltependocel only subjects showing a small decrease in the proportion.

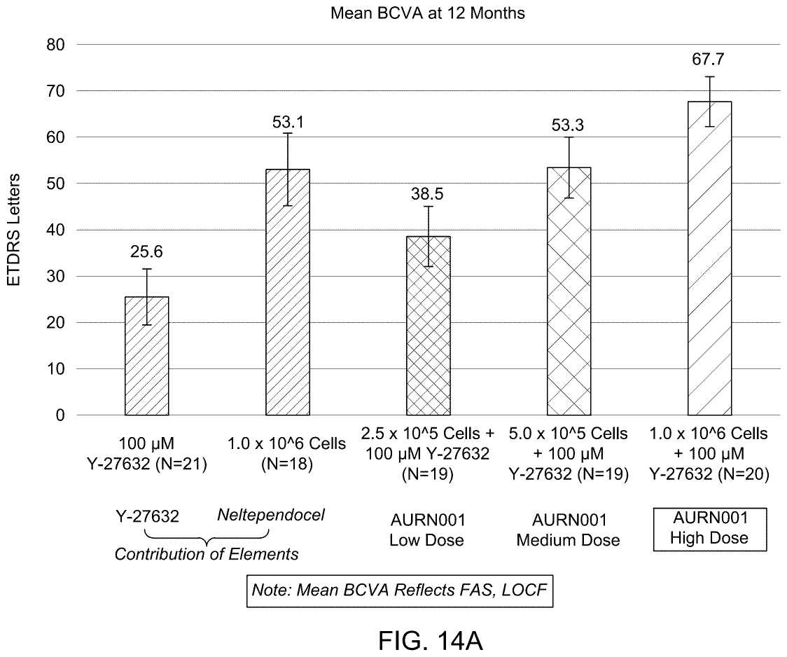

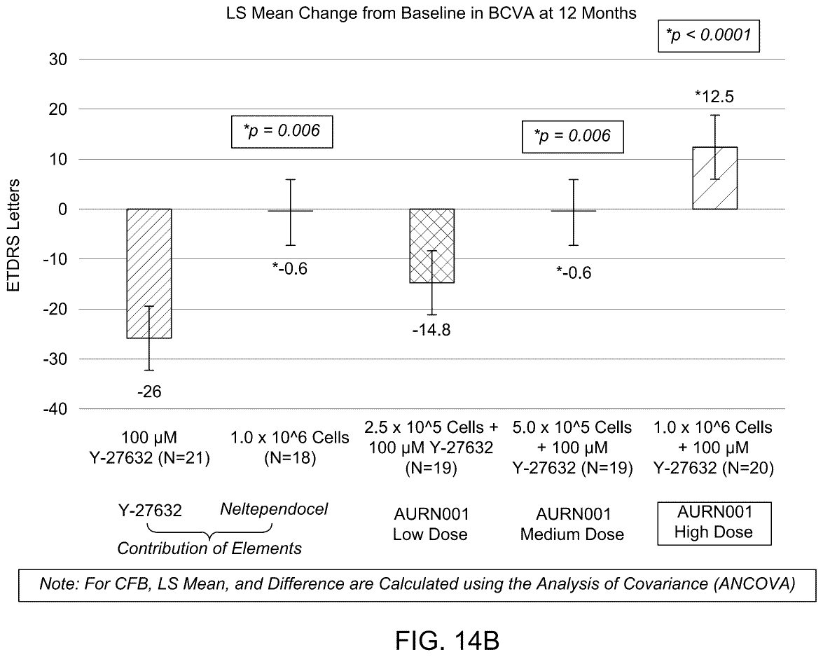

The mean BCVA and LS mean change from baseline BCVA at 12 months (FAS, LOCF) was also quantified. Subjects in the high-dose group demonstrated the highest mean BCVA (67.7 letters) at month 12 (FIG. 14A), and an improvement from the 65.8 letters at month 6 (FIG. 6). In comparison, all other treatment groups demonstrated decreased mean BCVA compared to month 6 analysis. Similarly, FIG. 14B shows that only subjects in the high-dose group exhibited a positive BCVA change from baseline at month 12, by 12.5 letters, while subjects in the other treatment groups experienced negative BCVA changes.

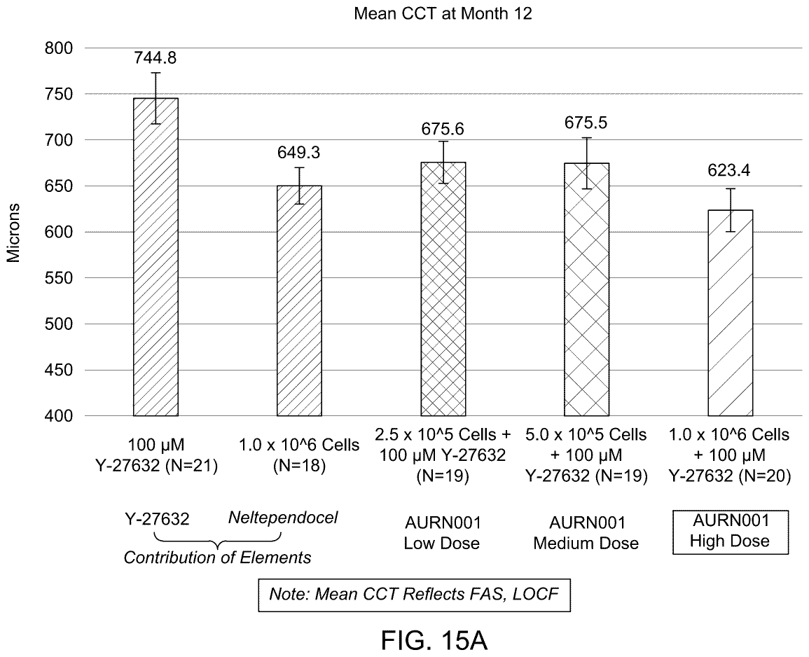

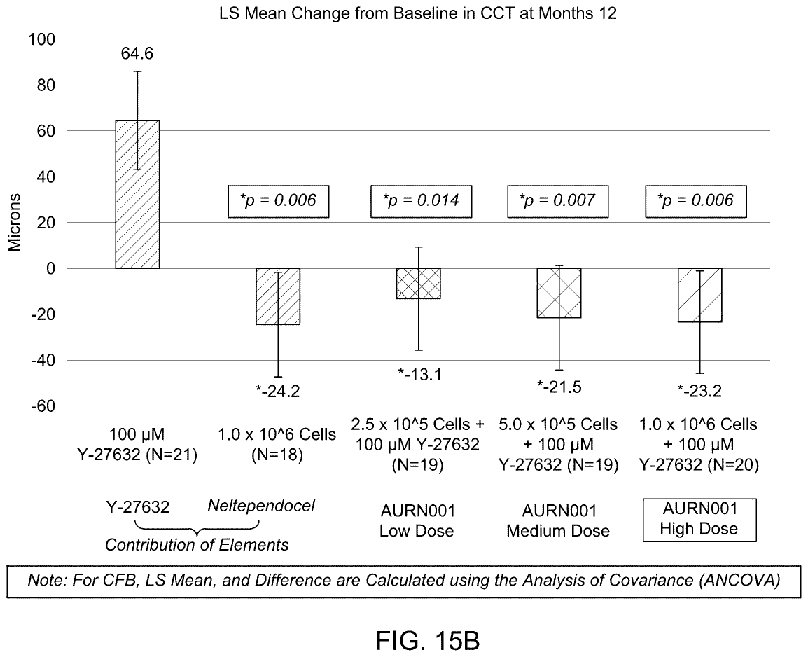

The efficacy of the various treatment regimens was also assessed by an alternate secondary efficacy endpoint of the study, i.e., the mean CCT and LS mean change from baseline CCT at 12 months (FAS, LOCF). The results are shown in FIGS. 15A-15B. A statistically significant (p=<0.05) outcome was achieved in all treatment groups for mean CCT changes from baseline relative to the Y-27632 Only Group. The high-dose group maintained the lowest mean CCT at month 12, and a second largest decrease in mean CCT, consistent with month 6 results.

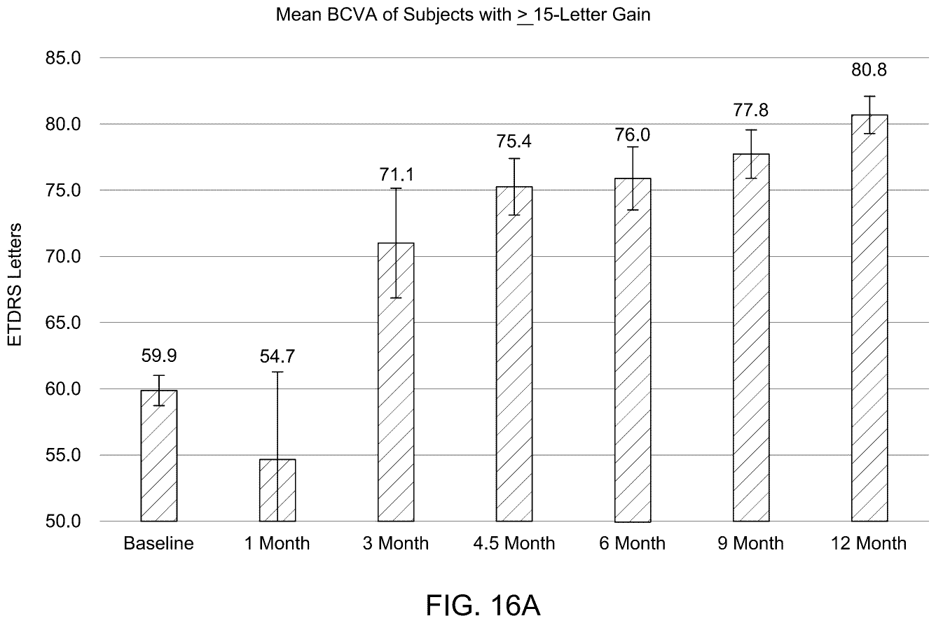

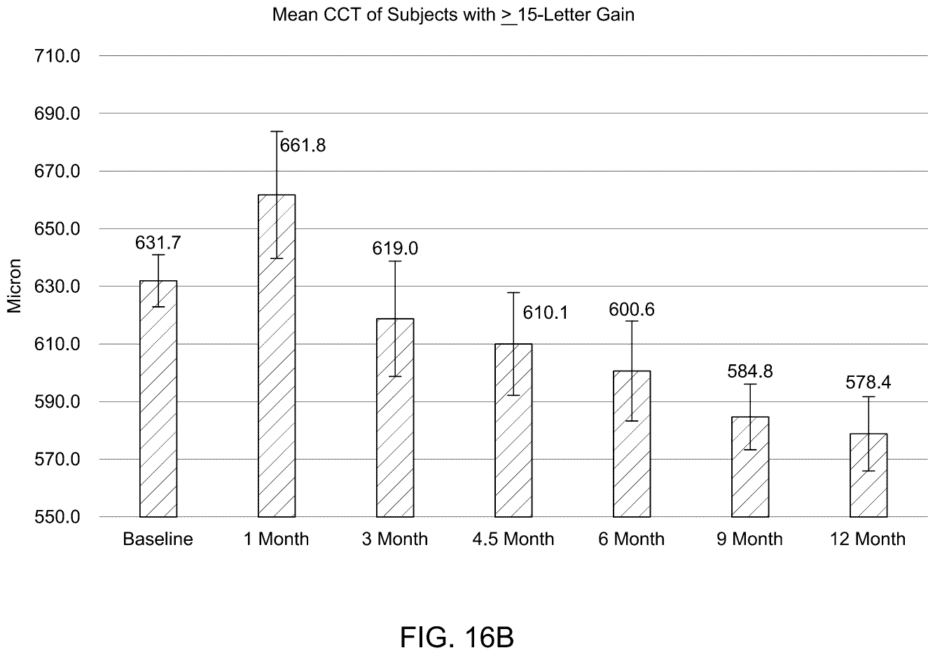

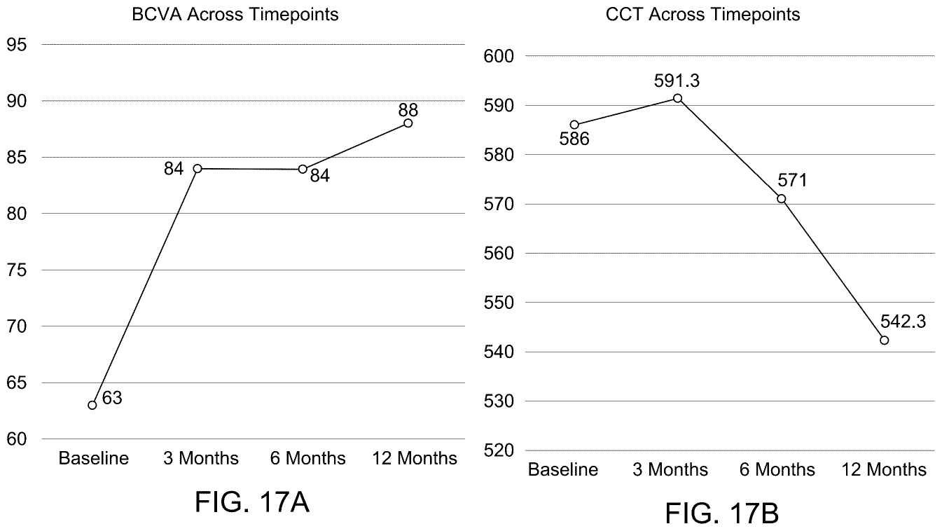

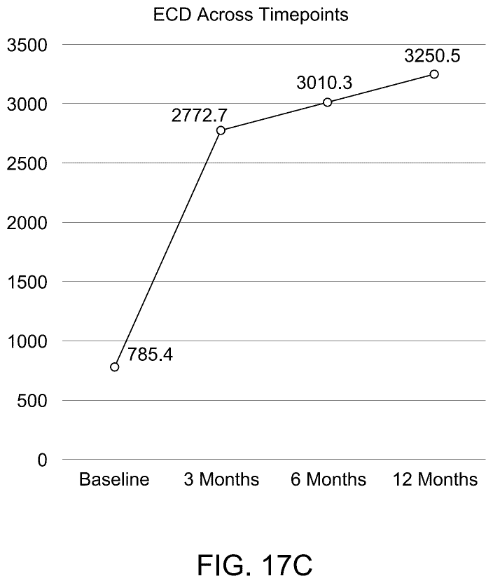

Additionally, subjects in the high-dose group that met the primary endpoint of >15-letter BCVA improvement at month 12 also demonstrated consistent BCVA improvements post treatment throughout the 12-month period (FIG. 16A) and consistent decreases in mean CCT (FIG. 16B). Overall, 65% of subjects achieved ≥15-letter improvement from baseline in BCVA at 12 months. In the meantime, Per Protocol Set (PPS) analysis, excluding subjects that had protocol deviations, yielded a percentage of 72% (i.e., 72% high-dose subjects met the BCVA primary endpoint). FIGS. 17A-17C demonstrate the changes in BCVA (FIG. 17A), CCT (FIG. 17B), and endothelial cell density (ECD) (FIG. 17C) for a representative subject in the high-dose group that met the primary endpoint. As shown, BCVA and ECD increased across timepoints and CCT decreased post-treatment.

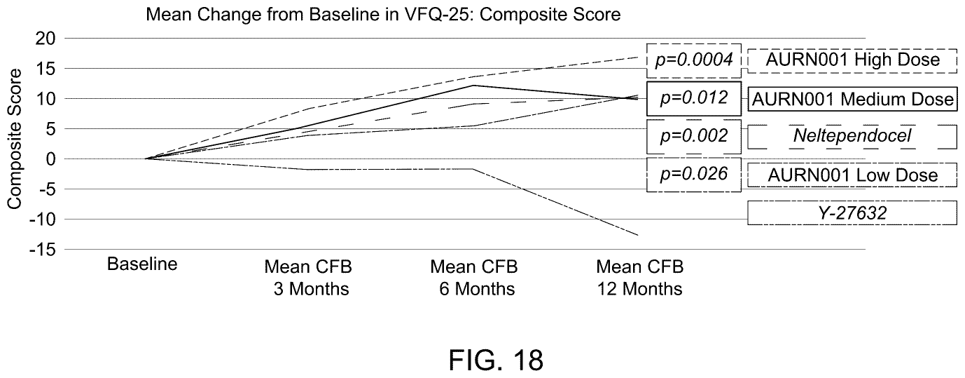

The efficacy of the various treatment regimens was also assessed by the patient reported outcomes as assessed using VFQ-25. Similar to month 6 analysis, month 12 analysis indicates that all treatment groups expect for Y-27632 only group resulted in positive improvement in VFQ-25 composite score, with the high-dose group having continuous improvements to month 12 (FIG. 18). In contrast, subjects in Y-27632 only group reported worsened self-reported outcomes reflected by the negative VFQ-25 change.

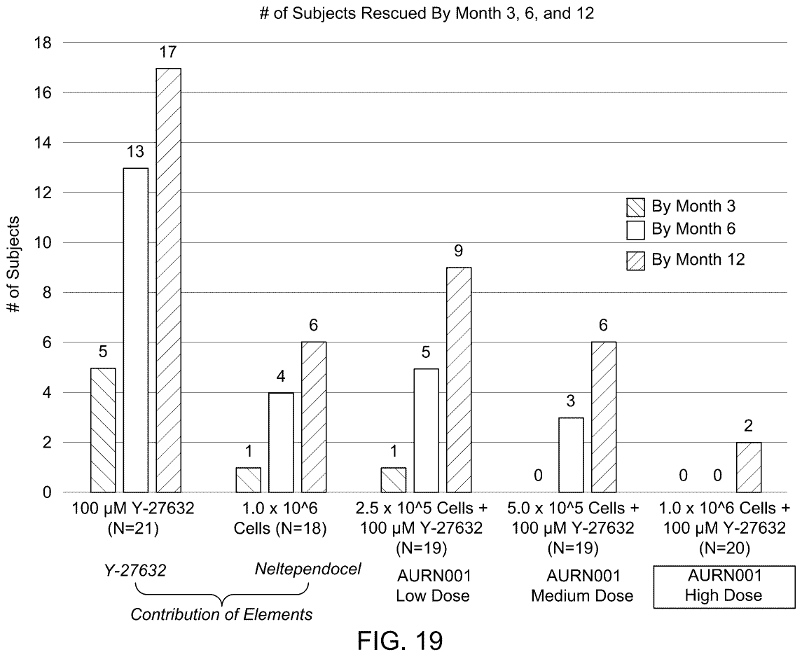

As shown in FIG. 19, subjects treated with cell therapy, either alone or in combination with 100 μM Y-27632 required fewer rescue surgeries at all of 3 month, 6 month, and 12 month time points as compared to subjects treated with 100 μM Y-27632 alone. Only two subjects treated with a combination of 1.0×10\hat{ }6 cells and 100 μM Y-27632 (high-dose) required rescue surgery during the 12 months of the study. Fewer subjects in the medium-dose group required rescue surgery than in the low-dose group at all time points, suggesting a dose-dependent effect.

### Safety Outcomes

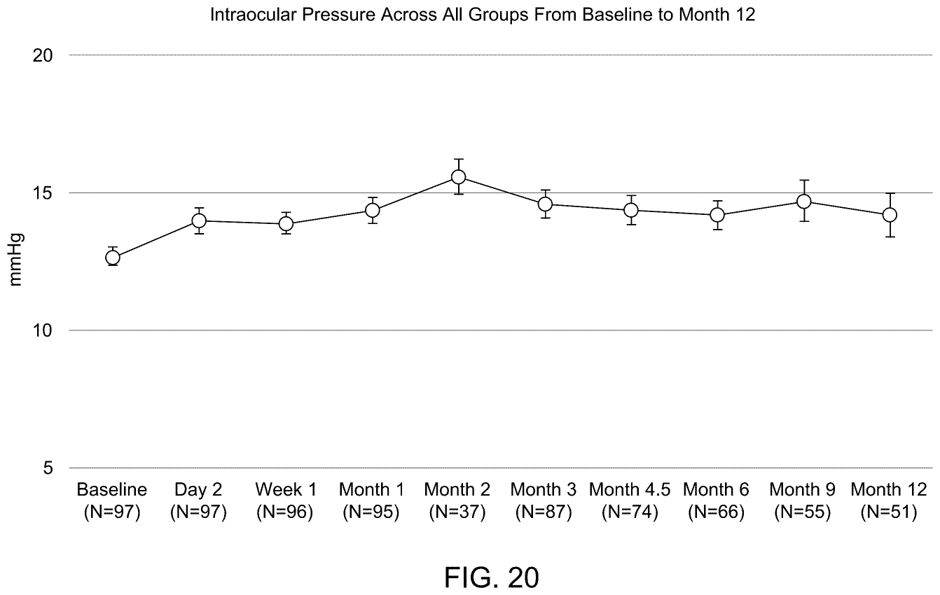

The results provided in FIG. 20 demonstrate the maintained safety profile of all of the treatment regimens. No significant changes in the intraocular pressure was observed across all groups from baseline to month 12. Of all subjects, the most common adverse event (>3%) were: ocular hypertension (10.3%; transient in nature and considered secondary to a steroid-induced response; lowest incidence in high-dose group), conjunctival hemorrhage (6.2%), epithelia defect (3.1%), ocular pain (3.1%), cystoid macular edema (3.1%), and corneal abrasion (3.1%). The most common non-ocular adverse events (>3%) were sinusitis (4.1%) and COVID-19 (3.1%).

Overall, Example 2 suggests that the high-dose group of AURN001 (1.0×10\hat{ }6 cells and 100 μM Y-27632) lead to long-lasting efficacy, the most improvement, and satisfactory safety profile maintained throughout the 12 month period post-treatment. AURN001 across doses were well tolerated with a favorable safety profile, and no graft rejections or severe adverse events.

### Example 3: Phase I Clinical Trial for AURN001

A Phase I prospective, randomized, double-masked, parallel-group clinical trial (the ESCALÓN trial) was performed to evaluate the safety and efficacy of doses of AURN001, a combination product comprising neltependocel and Y-27632, for treating corneal edema secondary to endothelial dysfunction.

The ESCALÓN trial was conducted at a single center in El Salvador. The study adhered to the guidelines set by the Declaration of Helsinki. The study protocol was reviewed and approved by the El Salvador National Research Ethics Committee, and all subjects provided written informed consent to participate in the study. The study was registered with ClinicalTrials.gov ID NCT 05309135.

### Inclusion Criteria Included:

Adults aged 18 years or older with a clinical diagnosis of corneal edema secondary to endothelial dysfunction;
BCVA between 0.5 and 1.6 log MAR in the study eye;
Pseudophakic with monofocal intraocular lens (IOL) implant located fully within the capsular bag;
Willing and able to lie face down for up to 3 hours immediately after the procedure; and Able to give written informed consent.

### Exclusion Criteria Included:

Keratoconus or other conditions of corneal thinning/ectasia;
Progressive stromal or anterior corneal dystrophies;
Prior ophthalmic surgery including corneal transplantation, glaucoma, vitreoretinal surgery, aphakia, and anterior chamber/iris claw/multifocal IOL;
Vitreous prolapse into the anterior segment; uncontrolled comorbidities of the ocular surface or anterior segment (including herpetic infections);
Uveitis or peripheral anterior synechiae, visually significant macular or intraretinal hemorrhage, retina or optic nerve pathology;
Relative Afferent Pupillary Defect in the study eye; or
Cognitive impairment or were unable to make independent decisions on study participation or had life-threatening or other diseases that would prevent participation in the study.

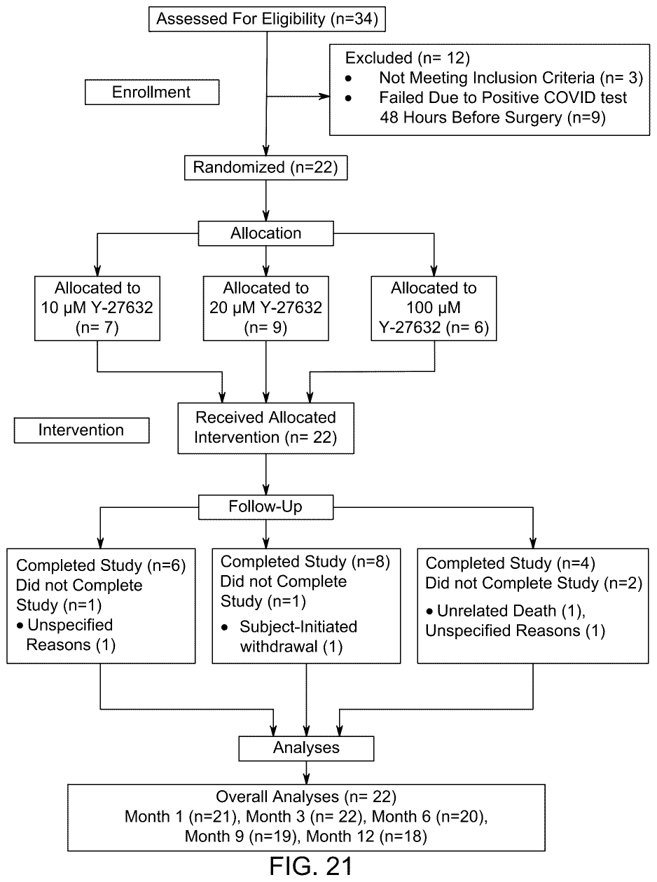

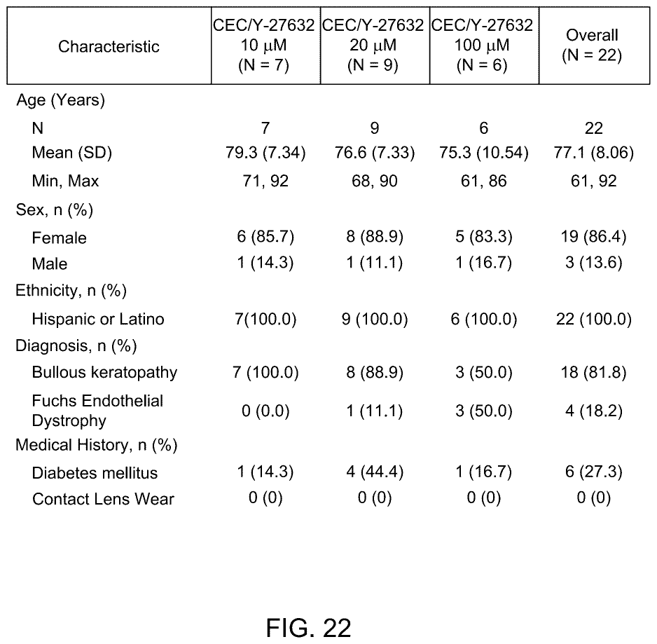

Eligible subjects with bullous keratopathy (N=18) or Fuchs dystrophy (N=4) were randomized to receive endothelial polishing, a single intracameral injection containing 1×10⁶ CECs (neltependocel), and 10, 20, or 100 μM Y-27632. Subjects were randomly assigned to the Y-27632 dosage groups (7 in the 10 μM Y-27632 group, 9 in the 20 μM group, and 6 in the 100 μM group). All subjects received 1×10⁶ CECs. The study design is shown in FIG. 21.

CECs (neltependocel) were cultured in Japan and shipped in culture vessel to El Salvador with temperature maintained between 2-8° C. On the day of surgery, cells were harvested and combined with 10, 20, or 100 μM Y-27632. The mixture product was stored between 2-8° C. before use, and administered within 6 hours from harvest.

The surgical procedure is similar to that described in Examples 1-2. On the day of treatment, a topical anesthetic agent was administered to the study eye. A supratemporal 1.2-mm clear limbocorneal incision was created using a keratome knife. The central 8-mm area of diseased endothelium was removed using a 27-gauge silicone-tipped polishing cannula. Endothelial removal and retention of Descemet membrane were confirmed with trypan dye. The anterior chamber was irrigated using BSS Plus® (Alcon) to clear residual dye and shallowed to accommodate cell suspension injection. The main incision was closed using a single 10-0 nylon suture. The cell suspension (300 μL) was injected intracamerally at a new entry point with a 27-gauge needle. Corticosteroids and antimicrobial agents were then administered topically to the corneal surface, and the eye was covered using a protective eye shield. The subject was immediately transferred to a prone position for 3 hours before discharge.

Follow-up assessments occurred on day 1, week 1, and months 1, 3, 6, 9, and 12. Key clinical and laboratory evaluations in this study, similar to those in Examples 1-2, included concomitant medications, noncontact and contact corneal pachymetry, corneal topography, ECD, anterior/posterior segment optical coherence tomography, manifest refraction, BCVA, brightness acuity test (BAT), slit-lamp exam, tonometry, gonioscopy, dilated fundus/posterior segment exam, and adverse events (AEs) recording. ECD was determined by masked staff at a Central Reading Center using images from noncontact specular microscopy (Konan NSP-9900). The primary outcome was safety based on incidence and severity of ocular and nonocular treatment-emergent adverse events (TEAEs). Efficacy outcomes included changes from baseline in central corneal thickness (CCT) and best-corrected visual acuity (BCVA) at all time points up to month 12.

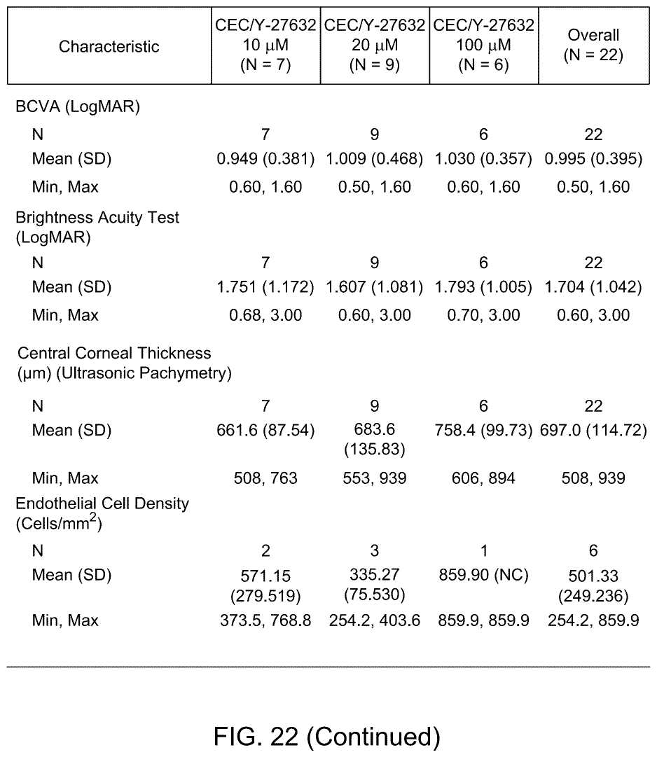

The demographics for each group and the population overall, and the baseline BCVA, baseline CCT, and baseline brightness acuity test (BAT) are provided in FIG. 22. Eighteen subjects (82%) completed the study; reasons for early study exit included unrelated death (1), subject-initiated withdrawal (1), and unspecified reasons (2). Nineteen of the 22 subjects (86.4%) remained in the study for at least 9 months. Because of significant corneal edema preoperatively, baseline ECD data were only available from 6/22 subjects. The mean ECD assessed by the Central Reading Center was 501.33 cells/mm² (range: 254.2-859.9 cells/mm²) in the study eye. Baseline characteristics were comparable among the three groups.

### Safety Outcomes

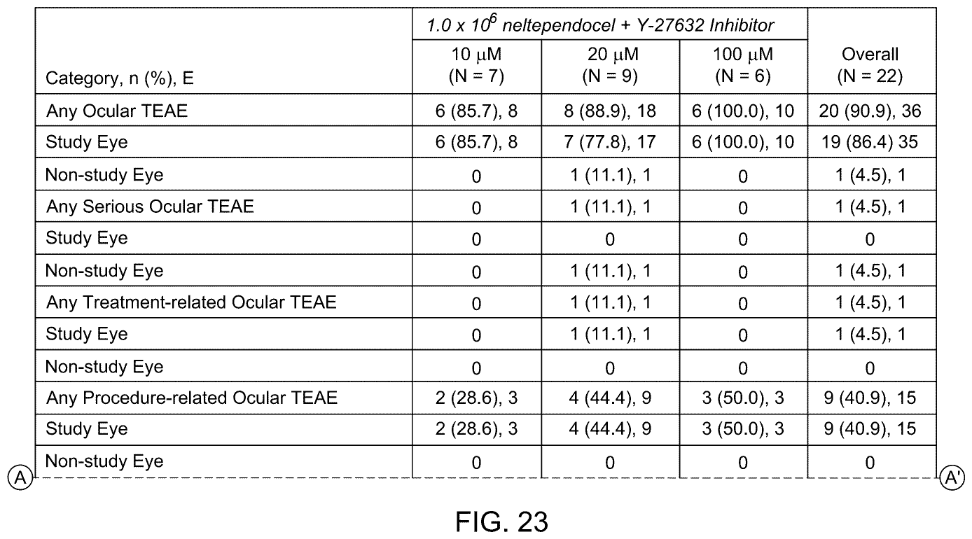

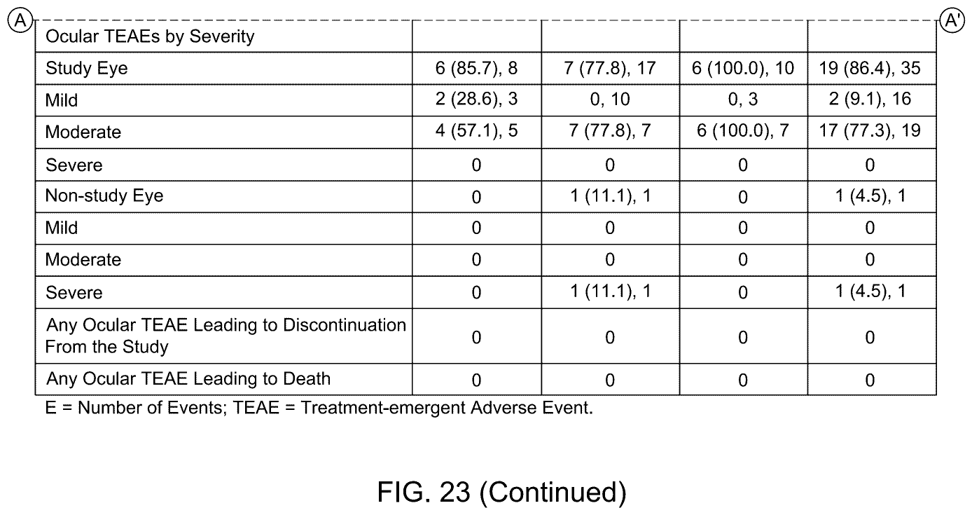

No cases of graft rejection were observed in ESCALÓN study. Thirty-six TEAEs occurred in 20 subjects, 35 of which occurred in the study eyes of 19 subjects and one in the nonstudy eye of one subject (bacterial keratitis) (FIG. 23). Fifteen procedure-related TEAEs occurred in nine subjects. No SAEs occurred in the study eyes; a single SAE (bacterial keratitis) occurred in a nonstudy eye and was deemed unrelated to treatment or procedure. No ocular TEAEs were reported that led to subjects discontinuing the study. Two nonocular TEAEs were reported, neither was related to the study treatment or procedure. These included one fatal case of myocardial infarction (SAE) and one case of nonserious, mild hypertensive crisis.

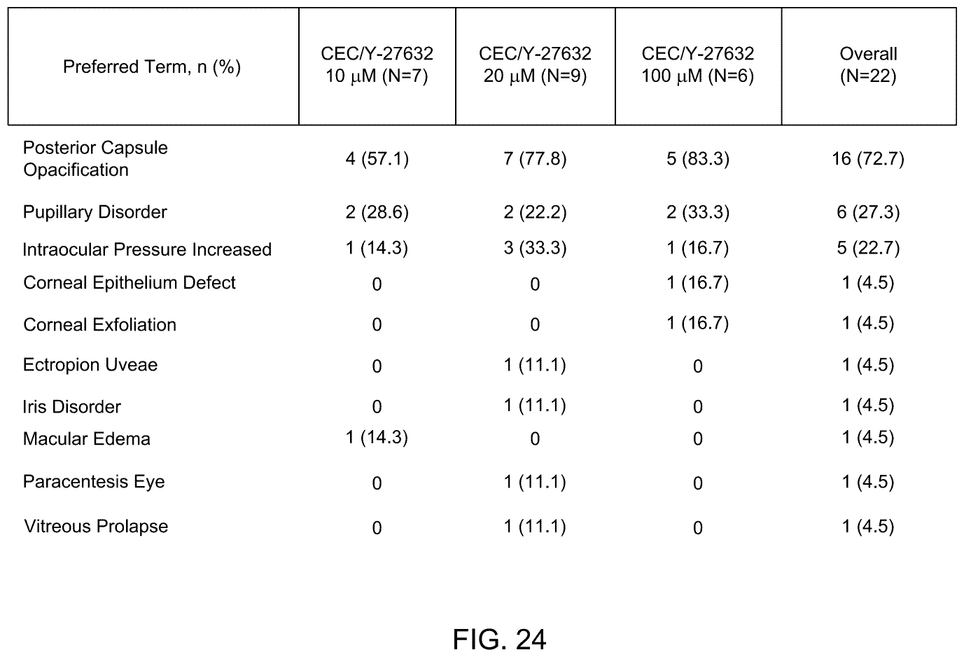

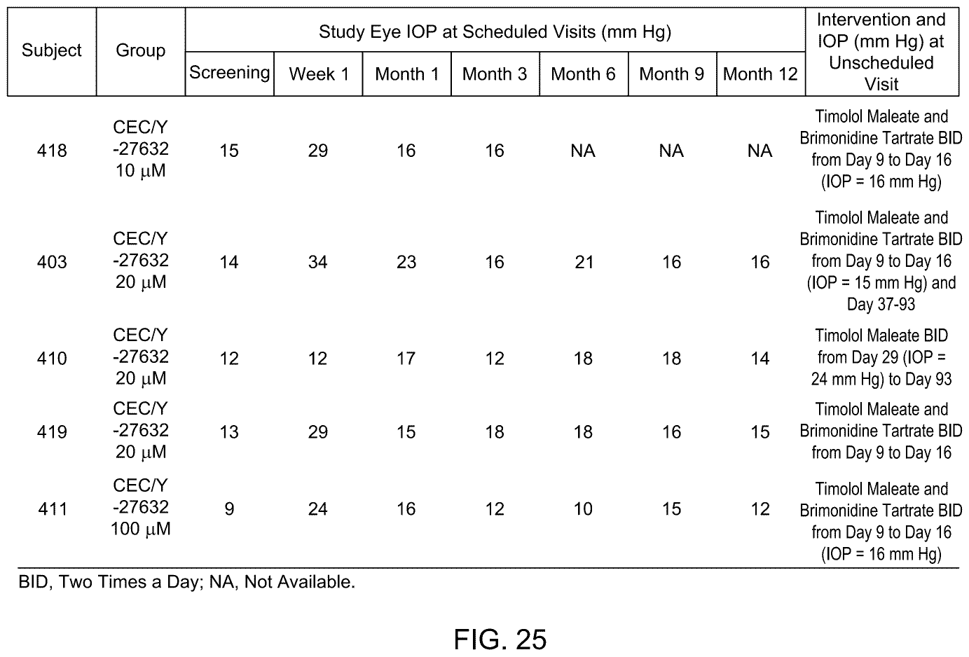

The nature and incidence of TEAEs in ESCALÓN study eyes are shown in (FIG. 24). The most common TEAEs were posterior capsule opacification (PCO) in 16/22 eyes (72.7%), pupillary disorders (dyscoria) in 6/22 eyes (27.3%), and transient intraocular pressure (IOP) elevations in 5/22 eyes (22.7%). Severe corneal edema at baseline hampered the detection of PCO in most study eyes; subjects with PCO underwent YAG capsulotomy after the 6-month follow-up visit. All cases of increased IOP were considered by the investigator to be related to the study procedure or steroid medication. One subject in the 10 μM group had controlled glaucoma at baseline without surgical intervention. Elevated IOP in this study was managed with topical IOP-lowering agents, which were discontinued upon the return of IOP to baseline levels (FIG. 25). No dose-dependent relationship for TEAE was observed across the study cohorts.

Preoperative corneal edema impaired visibility and hindered the accurate baseline assessment of intraocular structures in several subjects, including the pupil, iris, and lens. Throughout the study, observed pupillary/iris changes were primarily mechanical in nature, stemming from preoperative atrophic irides, shallow anterior chambers, and surgical manipulation as assessed. There were six cases of pupillary disorder (two in each treatment group), one case of ectropion uveae, and one case of iris disorder (both in the 20 μM group). All cases were mild, asymptomatic, and unaccompanied by visual disturbance, ocular dysphotopsia, or photophobia. No significant perioperative complications were observed in association with endothelial cell removal.

### Efficacy Outcomes

### Central Corneal Thickness (CCT)

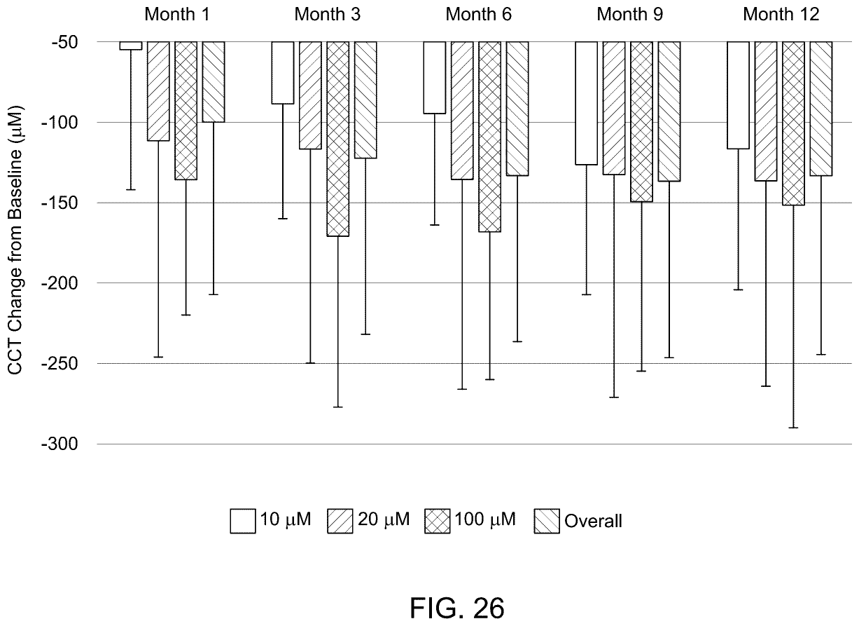

In the overall population, the mean (SD) CCT in the study eye was 697.0 (114.72) microns at baseline, which improved to 598.1 (82.03) microns by month 1, 574.1 (77.74) microns by month 3, 574.1 (74.94) microns by month 6, 570.3 (74.09) microns by month 9, and to 571.2 (88.82) microns by month 12. Mean (%) change in CCT from baseline was −100 (−12.95%) microns at month 1, −122.9 (−16.26%) microns at month 3, −133.5 (−17.71%) microns at month 6, −136.9 (−18.03%) microns at month 9, and −133.4 (−17.74%) microns at month 12. Changes in CCT were observed as early as month 1 and persisted throughout the 12-month study duration, with a trend toward greater improvement with the higher Y-27632 doses, although interpretation was limited by nonequivalent baseline values between treatment groups (FIG. 26). At month 12, the mean (%) change in CCT from baseline was −116.7 (−16.53%) microns in the 10 μM group, −136.6 (−17.55%) microns in the 20 μM group, and −152.3 (−19.92%) microns in the 100 μM group.

### Best-Corrected Visual Acuity (BCVA)

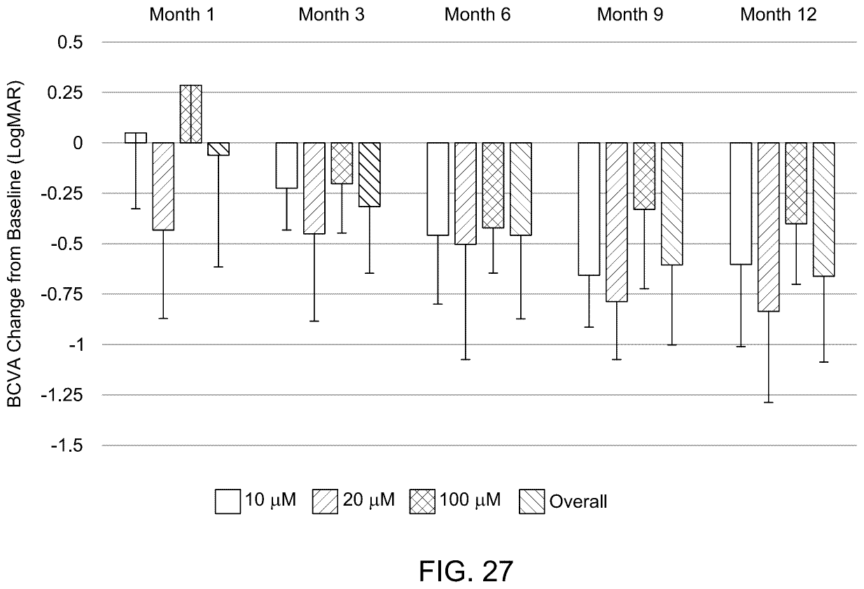

In the overall population, the mean (SD) BCVA at baseline was 0.995 (0.395) log MAR, which improved to 0.945 (0.687) log MAR at month 1, 0.683 (0.409) log MAR at month 3, 0.567 (0.455) log MAR at month 6, 0.407 (0.369) log MAR at month 9, and 0.330 (0.318) log MAR at month 12. Mean (SD) improvement in BCVA was 0.068 (0.548) log MAR at month 1, 0.313 (0.333) log MAR at month 3, 0.466 (0.406) log MAR at month 6, 0.612 (0.391) log MAR at month 9, and 0.662 (0.423) log MAR at month 12 (FIG. 27). BCVA showed notable improvement by month 3 and continued to improve up to the end of the study at month 12. No dose-response relationship was observed between BCVA and Y-27632 concentration. At month 12, the mean (SD) change from baseline in BCVA was −0.603 (0.407) log MAR in the 10 μM group, −0.835 (0.450) log MAR in the 20 μM group, and −0.405 (0.297) log MAR in the 100 μM group.

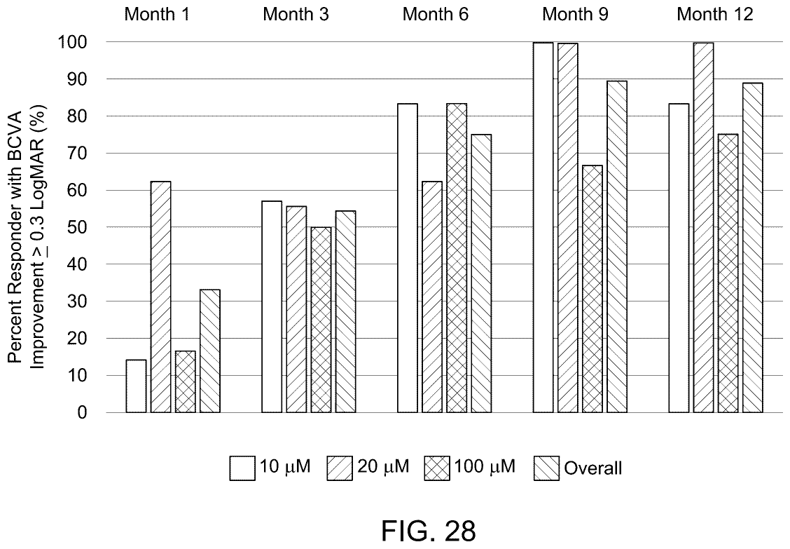

In the overall population, the proportion of all subjects who had a ≥0.3 LogMAR (3-line) score improvement in BCVA was 33.3% at month 1 (N=21), 54.5% at month 3 (N=22), 75.0% at month 6 (N=20), 89.5% at month 9 (N=19), and 88.9% at month 12 (N=18) (FIG. 28). No dose-response relationship was observed between BCVA and Y-27632 concentration. At month 12, the proportion of all subjects who had a ≥0.3 LogMAR (3-line) score improvement in BCVA was 83.3% in the 10 μM group, 100% in the 20 μM group, and 75% in the 100 μM group.

### Brightness Acuity Testing (BAT)

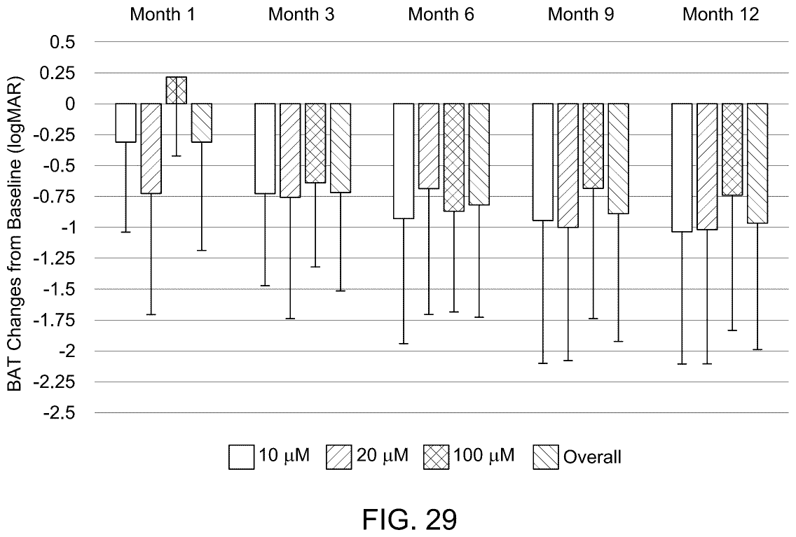

The improvements observed in BAT were comparable to the changes in BCVA. In the overall population, mean (SD) BAT was 1.704 (1.042) log MAR at baseline, which improved to 1.417 (1.075) log MAR at month 1, 0.981 (0.801) log MAR at month 3, 0.960 (0.543) log MAR at month 6, 0.823 (0.358) log MAR at month 9, and 0.754 (0.270) log MAR at month 12. These changes represent improvements of −0.328 (0.863) log MAR at month 1, −0.723 (0.798) at month 3, −0.823 (0.913) log MAR at month 6, −0.895 (1.034) at month 9, and −0.970 (1.024) log MAR at month 12 (FIG. 29). The changes in BAT were evident by the first 1 to 3 months and continued to improve throughout the 12-month study period. No dose-response relationship was observed between Y-27632 concentration and BAT improvement. At month 12, the mean (SD) change from baseline in BAT was −1.047 (1.067) log MAR in the 10 μM group, −1.025 (1.087) log MAR in the 20 μM group, and −0.745 (1.094) log MAR in the 100 μM group.

### Endothelial Cell Density (ECD)

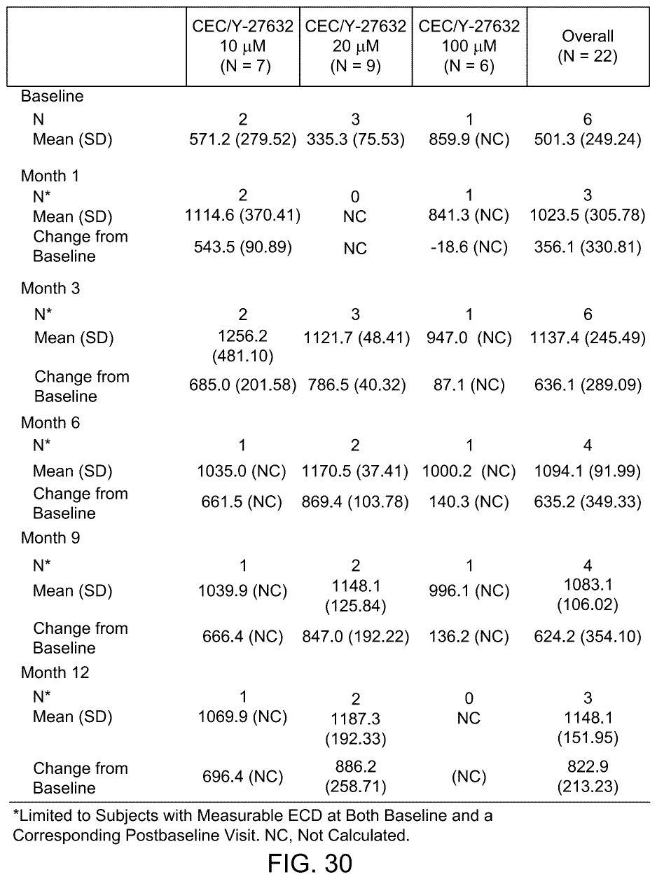

Endothelial cell density (ECD) was assessed using a noncontact specular microscope (Konan NSP-9900). Severe preoperative corneal edema prevented baseline ECD measurements in 16 out of 22 subjects. In the six subjects with measurable ECD data, the mean (SD) ECD at baseline was 501.3 (249.24) cells/mm² (FIG. 30). Post-procedure, overall ECD values improved to 1023.5 (305.78) cells/mm² at month 1 (N=3), 1137.4 (245.49) cells/mm² at month 3 (N=6), 1094.1 (91.99) cells/mm² at month 6 (N=4), 1083.1 (106.02) cells/mm² at month 9 (N=4), and 1148.1 (151.95) cells/mm² at month 12 (N=3). These results represented overall improvements of 356.1 (330.81), 636.1 (289.09), 635.2 (349.33), 624.2 (354.10), and 822.9 (213.23) cells/mm² at months 1, 3, 6, 9, and 12, respectively. Although the small sample size limited interpretation, ECD showed a favorable upward trend at 1, 3, 6, 9, and 12 months after intervention.

Overall, Example 3 suggests that corneal endothelial cell therapy (neltependocel and Y-27632) was well tolerated, with a favorable safety profile among the three Y-27632 dose groups evaluated. The nature and incidence of AEs were similar across the Y-27632 dosing groups with no apparent dose-response relationship. Data also provided evidence supporting the efficacy of corneal endothelial cell therapy (neltependocel and Y-27632) in several key measures, including BCVA, BAT, and CCT.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the disclosure have been discussed, the above specification is illustrative and not restrictive. Many variations of the disclosure will become apparent to those skilled in the art upon review of this specification and the claims below. The full scope of the disclosure should be determined by reference to the claims, along with their full scope of equivalents, and the specification, along with such variations.

1. A method for treating a corneal endothelial disease in a human subject in need thereof, comprising administering an effective amount of a corneal endothelial cell (CEC) composition and an effective amount of a Rho-associated, coiled-coil containing protein kinase (ROCK) inhibitor to the human subject, wherein
the CEC composition comprises allogeneic human CECs,
the CEC composition is stored at a temperature between about 2° C. and about 8° C. for a period of time before administration,
the period of time is about 3 hours to about 72 hours; optionally wherein the period of time is about 6 hours to about 48 hours, about 12 hours to about 48 hours, or about 24 hours to about 48 hours.

2. (canceled)

3. The method of claim 1, wherein the ROCK inhibitor is Y-27632, optionally wherein the effective amount of the ROCK inhibitor is about 10 μM to about 500 μM, about 50 μM to about 250 μM, about 80 μM to about 120 μM, or about 100 μM; further optionally wherein the effective amount of the ROCK inhibitor is about 100 μM.

4. The method of claim 1, wherein the CEC composition comprises Dulbecco's modified eagle medium (DMEM); optionally wherein the CEC composition further comprises human serum albumin (HSA).

5. The method of claim 1, wherein
(i) at least 75% of the allogeneic human CECs are CD166 positive, CD105 negative to weakly positive, and CD44 negative to weakly positive;
(ii) at least 75% of the allogeneic human CECs are CD166 positive, CD105 negative, and CD44 negative;
(iii) at least 75% of the allogeneic human CECs are CD24 negative, CD166 positive, CD105 negative to weakly positive, and CD44 negative to weakly positive;
(iv) at least 75% of the allogeneic human CECs are CD24 negative, CD166 positive, CD105 negative, and CD44 negative;
(v) at least 75% of the allogeneic human CECs are CD26 negative, CD166 positive, CD105 negative to weakly positive, and CD44 negative to weakly positive; or
(vi) at least 75% of the allogeneic human CECs are CD26 negative, CD166 positive, CD105 negative, and CD44 negative.

6-7. (canceled)

8. The method of claim 1, wherein the corneal endothelial disease is a bullous keratopathy, a corneal edema, a corneal leukoma, a corneal endothelial inflammation, or a corneal dystrophy (optionally, Fuchs' dystrophy).

9-11. (canceled)

12. A method for achieving an improvement from baseline in Best Corrected Visual Acuity (BCVA) at a timepoint post-treatment in a human subject with a corneal endothelial disease in need thereof, the method comprising administering to the human subject an effective amount of a corneal endothelial cell (CEC) composition and an effective amount of a Rho-associated, coiled-coil containing protein kinase (ROCK) inhibitor, wherein
the improvement is a ≥10-letter improvement in a Logarithm of the Minimum Angle of Resolution (LogMAR) chart or an equivalent improvement,
the timepoint post-treatment is at least about 6 months post-treatment, and
the CEC composition comprises allogeneic human CECs.

13. The method of claim 12, wherein the CEC composition is stored at a temperature between about 2° C. and about 8° C. for a period of time before administration, and the period of time is about 3 hours to about 72 hours; optionally wherein the period of time is about 6 hours to about 48 hours, about 12 hours to about 48 hours, or about 24 hours to about 48 hours.

14-15. (canceled)

16. The method of claim 12, wherein the improvement is a ≥15-letter improvement in a Logarithm of the Minimum Angle of Resolution (LogMAR) chart or an equivalent improvement; optionally wherein the improvement is a ≥15-letter improvement in the Early Treatment Diabetic Retinopathy Standard (ETDRS) visual acuity test.

17. The method of claim 12, wherein the method further achieves a reduced central corneal thickness (CCT) or an increased endothelial cell density (ECD) from baseline in the human subject at the timepoint post-treatment.

18. (canceled)

19. The method of claim 12, wherein the timepoint post-treatment is at least 7 months post-treatment, at least 8 months post-treatment, at least 9 months post-treatment, at least 10 months post-treatment, at least 11 months post-treatment, or at least 12 months post-treatment.

20. The method of claim 12, wherein the ROCK inhibitor is Y-27632, optionally wherein the effective amount of the ROCK inhibitor is about 10 μM to about 500 μM, about 50 μM to about 250 μM, about 80 μM to about 120 M, or about 100 μM; further optionally wherein the effective amount of the ROCK inhibitor is about 100 μM.

21. The method of claim 12, wherein the CEC composition comprises Dulbecco's modified eagle medium (DMEM); optionally wherein the CEC composition further comprises human serum albumin (HSA).

22. The method of claim 12, wherein
(i) at least 75% of the allogeneic human CECs are CD166 positive, CD105 negative to weakly positive, and CD44 negative to weakly positive;
(ii) at least 75% of the allogeneic human CECs are CD166 positive, CD105 negative, and CD44 negative;
(iii) at least 75% of the allogeneic human CECs are CD24 negative, CD166 positive, CD105 negative to weakly positive, and CD44 negative to weakly positive;
(iv) at least 75% of the allogeneic human CECs are CD24 negative, CD166 positive, CD105 negative, and CD44 negative;
(v) at least 75% of the allogeneic human CECs are CD26 negative, CD166 positive, CD105 negative to weakly positive, and CD44 negative to weakly positive; or
(vi) at least 75% of the allogeneic human CECs are CD26 negative, CD166 positive, CD105 negative, and CD44 negative.

23-24. (canceled)

25. A method for treating a corneal endothelial disease in a plurality of human subjects in need thereof, the method comprising administering to the plurality of human subjects an effective amount of a corneal endothelial cell (CEC) composition and an effective amount of a Rho-associated protein kinase (ROCK) inhibitor, wherein
the CEC composition comprises allogeneic human CECs, and
at least 50% of the plurality of subjects treated achieve >15-letter improvement (>3-line gain) from baseline in Best Corrected Visual Acuity (BCVA) using the Early Treatment Diabetic Retinopathy Standard (ETDRS) visual acuity test at a timepoint post-treatment.

26. The method of claim 25, wherein the CEC composition is stored at a temperature between about 2° C. and about 8° C. for a period of time before administration, and the first period of time is about 3 hours to about 72 hours.

27. The method of claim 26, wherein the period of time is about 6 hours to about 48 hours, about 12 hours to about 48 hours, or about 24 hours to about 48 hours.

28. (canceled)

29. The method of claim 25, wherein the timepoint post-treatment is at least 6 months post-treatment, at least 7 months post-treatment, at least 8 months post-treatment, at least 9 months post-treatment, at least 10 months post-treatment, at least 11 months post-treatment, or at least 12 months post-treatment.

30. The method of claim 25, wherein the ROCK inhibitor is Y-27632, optionally wherein the effective amount of the ROCK inhibitor is about 10 μM to about 500 μM, about 50 μM to about 250 μM, about 80 μM to about 120 μM, or about 100 μM; further optionally wherein the effective amount of the ROCK inhibitor is about 100 μM.

31. The method of claim 25, wherein the CEC composition comprises Dulbecco's modified eagle medium (DMEM); optionally wherein the CEC composition further comprises human serum albumin (HSA).

32. The method of claim 25, wherein
(i) at least 75% of the allogeneic human CECs are CD166 positive, CD105 negative to weakly positive, and CD44 negative to weakly positive;
(ii) at least 75% of the allogeneic human CECs are CD166 positive, CD105 negative, and CD44 negative;
(iii) at least 75% of the allogeneic human CECs are CD24 negative, CD166 positive, CD105 negative to weakly positive, and CD44 negative to weakly positive;
(iv) at least 75% of the allogeneic human CECs are CD24 negative, CD166 positive, CD105 negative, and CD44 negative;
(v) at least 75% of the allogeneic human CECs are CD26 negative, CD166 positive, CD105 negative to weakly positive, and CD44 negative to weakly positive; or
(vi) at least 75% of the allogeneic human CECs are CD26 negative, CD166 positive, CD105 negative, and CD44 negative.

33-35. (canceled)
